(19) United States
(12) Patent Application Publication
(10) Pub. No.: US 2005/0245444 A1
(43) Pub. Date: Nov. 3, 2005

(54) Method of using recombinant human antithrombin for neurocognitive disorders

(76) Inventors: Yann Echelard, Jamaica Plain, MA (US); Harry M. Meade, Newton, MA (US)

Correspondence Address: GTC BIOTHERAPEUTICS, INC., 175 CROSSING BOULEVARD, SUITE 410, FRAMINGHAM, MA, 01702, US

(21) Appl. No.: 10/835,836

(22) Filed: Apr. 30, 2004

(51) Int. Cl. C12Q 1/68; A61K 38/54; A01K 67/027

(52) U.S. Cl. 514/12; 800/6; 800/14

(57) ABSTRACT

The present invention provides for the production of recombinant human antithrombin (rhAT) for the treatment or prophylaxis of neurocognitive disorders typically associated with major surgical procedures. The recombinant processes of the current invention as well as more efficient methods of treatment, formulation and production have been developed to treat the incidence of neurocognitive problems associated with visuoconstruction, parieto-occipital watershed area injury, hypoperfusion, microemboli or other larger embolic factors and/or CABG procedures secondary to surgery.

### FIELD OF THE INVENTION

The present invention relates to the production of recombinant human antithrombin (rhAT) for use in the treatment or prevention of neurocognitive disorders typically associated with major surgical procedures. In particular the current invention provides for the production of transgenic mammal derived AT for the treatment or prophylaxis of neurocognitive disorders associated with surgical procedures.

### BACKGROUND OF THE INVENTION

The frequency and severity of central nervous system complications in patients undergoing cardiopulmonary bypass (CPB) have been documented in a large number of cases. In fact as many as 30% of all major surgery patients may have some problems, either temporary or longterm, with their neurocognitive abilities. These potential patients may also be substantially higher numbers of individuals over 60 years of age. For example, the risks of embolic neurologic complications and stroke in the population older than 70 years from a severely atherosclerotic ascending aorta are well documented. Moreover, while the majority of CPB patients do not experience perioperative stroke, a high incidence of more subtle central nervous system dysfunction has been demonstrated to persist for up to 5 year after surgery in a high percentage of patients (5 year decline documented in Newman et al. New England Journal of Medicine (2001) 344:395).

Age has also been shown to be one of the strongest predictors of subsequent neurologic complications (“sequelae”) in patients undergoing CPB or other major surgical procedures. The risk of embolic complications in the brain also increases in proportion to the degree of atherosclerosis in the ascending aorta, which is also age-related. Transesophageal echocardiography has been found to be only partly useful in diagnosing these lesions or in guiding follow-on surgical procedures in comparison with epiaortic imaging, which is more precise and less invasive. Transcranial Doppler and retinal fluorescein angiography have provided further evidence of microemboli during surgical procedures.

Neurological injury is a potentially devastating complication of surgery that results in a longer hospitalization, increased costs, and a substantially increased likelihood of death. Such injury can affect any level of the central nervous system, and its manifestations are broad, ranging from barely registrable neurocognitive dysfunction to frank stroke. Many variables have been found to be indicative or risk for perioperative neurological injury, but the predictive models are more useful for stroke risk than for neurocognitive dysfunction already in place. Strategies aimed at reducing neurological injury during cardiac surgery have focused, for the most part, on the technical aspects of cardiopulmonary bypass. Similar strategies have been discussed for other major surgical procedures. Surgical procedures such as carotid endarterectomy and cardiac surgery continues to be controversial relative to their potential for initiating neurocognitive disfunction.

Cerebral embolism, including atheroembolism from the ascending aorta, has an important role in the pathogenesis of neurological injury of all types. Epiaortic ultrasound imaging of the aorta is a sensitive technique for the identification of atherosclerosis of the ascending aorta at the time of surgery, which can allow it to be avoided and therefore reduce the risk for atheroembolism. Results of laboratory investigations have provided insight into the mechanisms of ischemic neuronal injury and a basis for the development of neuroprotective drugs. Neuroprotection may best be accomplished during major surgical procedures, especially heart surgery because, in contrast to nonsurgical situations, potential agents can be administered before the neurological insult occurs. Neuroprotective procedures and thereapeutic agents may be effective in reducing the neurocognitive dysfunction associated with perioperative or post-operative stroke but it will require the assessment of risk factors and the determination by doctores and their patients about the need for the delivery of novel diagnostic and therapeutic strategies.

The search for clinically-effective neuroprotective agents has received enormous support in recent years—an estimated $200 million by pharmaceutical companies on clinical trials for traumatic brain injury since 1995. In fact, neurological complications are, at the present time, considered among the most important causes of morbidity and mortality after heart surgery. At the same time, the pathophysiology of brain injury has proved increasingly complex, rendering the likelihood of a single agent to prevent this damage even more remote. On the other hand, progress continues with technology that makes surgery less invasive and less risky. One example is the application of endovascular techniques to treat coronary artery stenosis, where both the invasiveness of sternotomy and the significant neurological complication rate, due to microemboli showering the cerebral vasculature, can be eliminated.

According to the current invention many patients who receive cardiopulmonary bypass experience lasting negative postoperative events. Four categories of neurologic outcome are of particular importance: (1) persistent neurological focal deficits, (2) stupor or coma, (3) temporary neurological focal deficits, and (4) seizures. Predictors of risk or neurobioligical complications were aortic aneurysm and aortic valve surgery, advanced age, female sex, and the use of intra-aortic balloon pump or angioplasty. Moreover, a significantly longer hospitalization time was noticed among patients with neurological side effects.

In addition, the current incidence of stroke during cardiopulmonary bypass is somewhat lower than in the 1980's but remains a significant problem today. Levels of cognitive impairment also are currently very high. Recognized predictors enable us to identify patients at particularly high risk of stroke. Hypertensive patients are particularly susceptible to ischemic injury during bypass and should be perfused at mean perfusion pressures higher than those for normotensive patients.

Cardiopulmonary bypass has been shown to activate various inflammatory cascades in the body, resulting in pathophysiological changes that may affect patient outcome after cardiac surgery. Many of these inflammatory cascades are enzyme mediated, involving serine proteases. The current invention reviews the mechanisms of bypass-mediated activation of the inflammatory cascades and outlines the role of serine protease inhibitors, particularly antithrombin and more specifically recombinant human antithrombin, in ameliorating the consequences of the inflammatory response. Data are reviewed on the action of aprotinin in inhibiting the intrinsic coagulation system and in limiting the contact activation of blood platelets and leukocytes. Also reviewed is the role of aprotinin in impacting the incidence of perioperative myocardial ischemia and the central nervous system dysfunction and stroke that are not infrequent complications of surgery with cardiopulmonary bypass.

The hemostatic system is an intricate system that maintains the fluidity of blood under normal physiologic conditions, yet reacts instantaneously to vascular injury to prevent blood loss by sealing the defect. Balanced stimulation and inhibition of the various mechanisms involved with hemostasis are fundamental to the physiologic state. Maintenance of this balance, under various pathologic and altered physiologic conditions, including cardiopulmonary bypass (CPB), remains a significant medical challenge. Blood contact with the extracorporeal circuit used in CPB provides a powerful stimulus to activate the hemostatic system. Anticoagulation is used during CPB to minimize activation of the hemostatic system and prevent thrombus formation, particularly in the extracorporeal circuit. Reversal of anti-coagulation at the end of CPB is important to minimize peri-operative blood loss and return the hemostatic system to its normal physiologic state. Well-controlled hemostatic management including rapid anti-coagulation and subsequent reversal is critical to the overall clinical outcomes of CPB.

Heparin therapy is used for anticoagulation during CPB, and other major surgical procedures, due to its rapid onset of action, ease of titration, and ability to be reversed. Heparin works by accelerating approximately 1000-fold the binding of antithrombin (generally, “AT III” or for the purposes of this invention recombinant human antithrombin “rhAT”) to thrombin and other coagulation factors. The binding of antithrombin to thrombin and other factors induces anticoagulation, prevents blood clot formation, and markedly inhibits activation of thrombin, factor Xa, and platelets. These interrelationships are illustrated in FIG. 2. Antithrombin binds thrombin in a one-to-one stoichiometric fashion. Thus, adequate AT III concentrations are critical to heparin's anticoagulant activity.

According to the current invention, when AT III concentrations decline below normal physiologic levels, decreased heparin anticoagulant response or heparin resistance may develop. Heparin resistance, or heparin “dysfunction” in surgical procedures due to a low level of rhAT, or its consequent pathological consequences may be broadly defined as the lack of normal anticoagulant response to heparin. The concept of heparin resistance has been applied to many clinical conditions where heparin is used. For example, heparin resistance when treating deep venous thrombosis (DVT) has been defined as the need for more than 35,000 U/day of heparin to maintain a therapeutic activated partial thromboplastin time (aPTT). In the setting of CPB, or other major surgical events, most patients develop adequate anticoagulation after 300 U/kg of heparin. Thus, patients who do not achieve adequate anticoagulation for CPB after a typical dose of heparin may be referred to as heparin resistant and may be in need of treatment with rhAT.

Antithrombin (“AT III”) is a serine protease inhibitor which inhibits thrombin and the activated forms of factors X, VII, IX, XI, and XII. It is normally present in serum at levels of 14-20 mg/dL. Decreased levels of AT III may be found in the serum of individuals who have either a hereditary deficiency of AT III or an acquired deficiency, which can result from a number of pathologic conditions. The conventional treatment for hereditary AT III deficiency is protein replacement therapy, which may also be effective in treating some acquired deficiencies.

Current methods of obtaining plasma-derived AT III involves isolating the protease inhibitor from blood plasma. However, the use of plasma-based AT III presents various problems due to the many components in plasma, including: variation between lots; immunogenicity problems; and biohazardous risks due to viral contamination. Therefore, a need exists to develope a method to produce recombinant antithrombin without the inherent problems and risks of the present process.

Accordingly, the recombinant processes of the current invention as well as more efficient methods of treatment, formulation and production are needed to treat the incidence of neurocognitive problems associated with the coagulation cascade and its associated pathologies. This may be accomplished, according to the current invention, through the use of rhAT in therapeutically effective amounts to reduce the incidence and severity of neurocognitive disorders associated with major surgical procedures preferably CPB.

### SUMMARY OF THE INVENTION

Briefly stated, the present invention relates generally to the production and therapeutic use of rhAT in the neurocognitive disorder field. As previously stated, cerebral injury is among the most common and disabling complications of open heart surgery and other major surgical events. Previous attempts to provide some level of neuroprotection have yielded conflicting and disappointing results. Acording to the current invention the therapeutic use of rhAT may ameliorate the observed neurocognitive problems.

One method of measuring problems with neurocognitive function is the measurement of neuron-specific enolase (NSE) and the S-100 protein. These proteins have been used as markers for major brain damage. Cognitive dysfunction after cardiac surgery represents subtle brain damage that is detected by neuropsychological testing. Therefore, one objective of the current invention is to use rhAT to therapeutically treat neurocognitive disorders that are or may prospectively be caused by major surgical procedures and to monitor such improvements according to the NSE and/or S-100 measurements.

The development of coronary artery bypass grafting (CABG) and its effect on angina is the product of a series of technical and scientific advances. Despite these advances, however, substantial numbers of adverse neurobehavioural outcomes continue to occur. Stroke is the most serious complication of CABG, but studies that have identified demographic and medical risk factors available before surgery are an important advance. Short-term neurocognitive deficits are common after a CABG procedure, but are not be specific to this procedure. Deficits in some cognitive areas such as visuoconstruction persist over time, and may reflect parieto-occipital watershed area injury secondary to hypoperfusion, microemboli or other larger embolic factors. According to the invention antithrombin, preferably rhAT, may be used therapeutically to lessen the disabilities suffered by surgical or CABG patients.

This invention is also directed to pharmaceutical compositions which comprise an amount of a transgenically produced protein of interest, a prodrug thereof, or a pharmaceutically acceptable salt of said compound or of said prodrug and a pharmaceutically acceptable vehicle, diluent or carrier useful in the treatment of neurocognitive disorders.

These and other objects which will be more readily apparent upon reading the following disclosure may be achieved by the present invention.

An additional objective of the current invention is to provide rhAT for the treatment and improvement in pulmonary function as assessed by PaO₂/FiO₂ ratio, reduce the number of ventilator days/% TBSA and reduce the number of ventilator days/length of hospital stay.

Another objective of the current invention is to decrease the incidence of ARDS and/or pneumonia while a patient is on the ventilator through the use of rhAT.

The present invention further relates to a drug composition for thrombotic disorders which contains the human AT III mutant according to the present invention and pharmaceutically acceptable carriers, a use of the human AT III mutant according to the present invention for the making of a medicament for treating thrombotic disorders, and a method for treating thrombotic disorders which comprises administering a pharmaceutically effective amount of the human AT III mutant according to the present invention to a patient suffering from the thrombotic disorders.

Further scope and the applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 Shows a Generalized Flowchart of the Process of Creating Cloned Animals through Nuclear Transfer.

FIG. 2 Shows a Thrombin Generation and/or Activation Cascade.

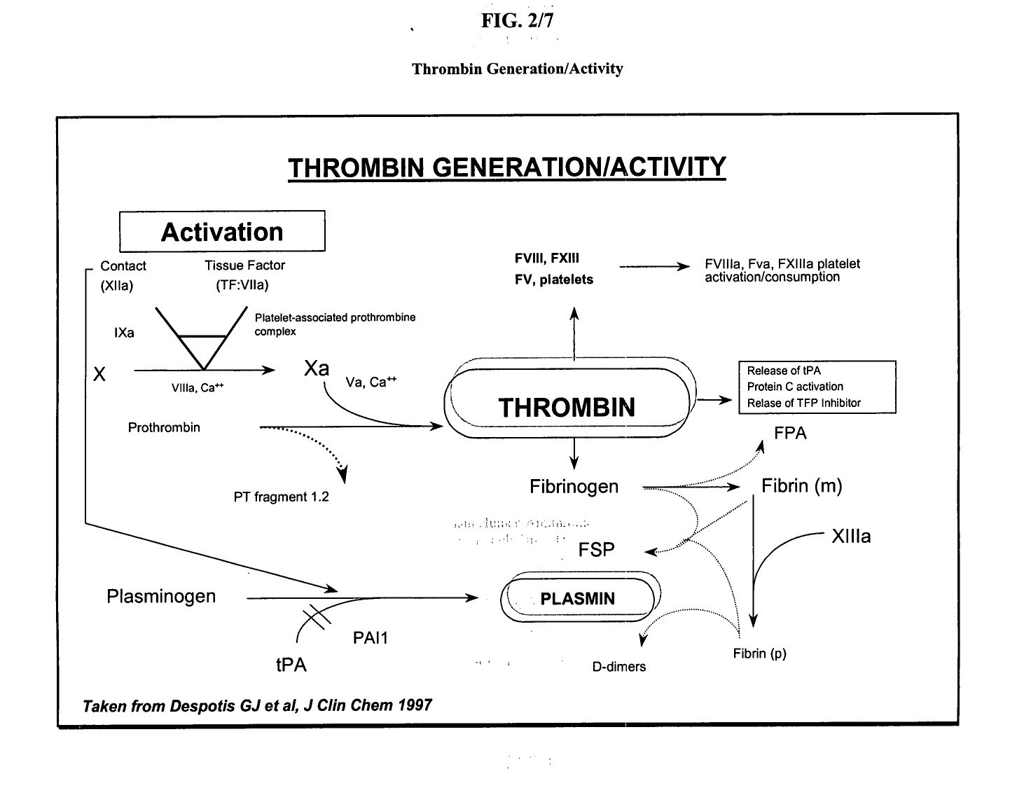

FIG. 3 Shows Timetable and Dosage for the Administration of Heparin, rhAT, and FFP.

FIG. 4 Shows Individual Patient Fibrin Monomer Plasma Levels Over Time.

FIG. 5 Shows Individual Patient AT III Plasma Activity Levels Over Time.

FIG. 6 Shows Individual Prothrombin Fragment 1.2 Plasma Levels Over Time for the rhAT without FFP and Placebo with FFP Cohorts.

FIG. 7 Shows Thrombin-Antithrombin Complex Plasma Levels Over Time for the rhAT without FFP and Placebo with FFP Cohorts

### DETAILED DESCRIPTION

The following abbreviations have designated meanings in the specification:

Explanation of Terms:

Bovine—Of or relating to various species of cows.
Biological Fluid—an aqueous solution produced by an organism, such as a mammal, bird, amphibian, or reptile, which contains proteins that are secreted by cells that are bathed in the aqueous solution. Examples include: milk, urine, saliva, seminal fluid, vaginal fluid, synovial fluid, lymph fluid, amniotic fluid, blood, sweat, and tears; as well as an aqueous solution produced by a plant, including, for example, exudates and guttation fluid, xylem, phloem, resin, and nectar.
Biological-fluid producing cell—A cell that is bathed by a biological fluid and that secretes a protein into the biological fluid.
Biopharmaceutical—shall mean any medicinal drug, therapeutic, vaccine or any medically useful composition whose origin, synthesis, or manufacture involves the use of microorganisms, recombinant animals (including, without limitation, chimeric or transgenic animals), nuclear transfer, microinjection, or cell culture techniques.
Caprine—Of or relating to various species of goats.
Encoding—refers generally to the sequence information being present in a translatable form, usually operably linked to a promoter (e.g., a beta-casein or beta-lacto globulin promoter). A sequence is operably linked to a promoter when the functional promoter enhances transcription or expression of that sequence. An anti-sense strand is considered to also encode the sequence, since the same informational content is present in a readily accessible form, especially when linked to a sequence which promotes expression of the sense strand. The information is convertible using the standard, or a modified, genetic code.
Expression Vector—A genetically engineered plasmid or virus, derived from, for example, a bacteriophage, adenovirus, retrovirus, poxvirus, herpesvirus, or artificial chromosome, that is used to transfer an obesity related transgenic protein coding sequence, operably linked to a promoter, into a host cell, such that the encoded recombinant obesity related transgenic protein is expressed within the host cell.
Functional Proteins—Proteins which have a biological or other activity or use, similar to that seen when produced endogenously.
Homologous Sequences—refers to genetic sequences that, when compared, exhibit similarity. The standards for homology in nucleic acids are either measures for homology generally used in the art or hybridization conditions. Substantial homology in the nucleic acid context means either that the segments, or their complementary strands, when compared, are identical when optimally aligned, with appropriate nucleotide insertions or deletions, in at least about 60% of the residues, usually at least about 70%, more usually at least about 80%, preferably at least about 90%, and more preferably at least about 95 to 98% of the nucleotides. Alternatively, substantial homology exists when the segments will hybridize under selective hybridization conditions, to a strand, or its complement. Selectivity of hybridization exists when hybridization occurs which is more selective than total lack of specificity. Typically, selective hybridization will occur when there is at least about 55% homology over a stretch of at least about 14 nucleotides, preferably at least about 65%, more preferably at least about 75%, and most preferably at least about 90%.
Leader sequence or a “signal sequence”—a nucleic acid sequence that encodes a protein secretory signal, and, when operably linked to a downstream nucleic acid molecule encoding a transgenic protein and directs secretion. The leader sequence may be the native human leader sequence, an artificially-derived leader, or may obtained from the same gene as the promoter used to direct transcription of the transgene coding sequence, or from another protein that is normally secreted from a cell.
Milk-producing cell—A cell (e.g., a mammary epithelial cell) that secretes a protein into milk.
Milk-specific promoter—A promoter that naturally directs expression of a gene in a cell that secretes a protein into milk (e.g., a mammary epithelial cell) and includes, for example, the casein promoters, e.g., α-casein promoter (e.g., alpha S-1 casein promoter and alpha S2-casein promoter), β-casein promoter (e.g., the goat beta casein gene promoter (DiTullio, BIOTECHNOLOGY 10:74-77, 1992), γ-casein promoter, and κ-casein promoter; the whey acidic protein (WAP) promoter (Gorton et al., BIOTECHNOLOGY 5: 1183-1187, 1987); the β-lactoglobulin promoter (Clark et al., BIOTECHNOLOGY 7: 487-492, 1989); and the α-lactalbumin promoter (Soulier et al., FEBS LETTS. 297:13, 1992). Also included are promoters that are specifically activated in mammary tissue and are thus useful in accordance with this invention, for example, the long terminal repeat (LTR) promoter of the mouse mammary tumor virus (MMTV).
Nuclear Transfer—This refers to a method of cloning wherein the nucleus from a donor cell is transplanted into an enucleated oocyte.
Operably Linked—A gene and one or more regulatory sequences are connected in such a way as to permit gene expression when the appropriate molecules (e.g., transcriptional activator proteins) are bound to the regulatory sequences.
Ovine—Of or relating to or resembling sheep.
Parthenogenic—The development of an embryo from an oocyte without the penetration of sperm.
Pharmaceutically Pure—This refers to transgenic protein that is suitable for unequivocal biological testing as well as for appropriate administration to effect treatment of a human patient. Substantially pharmaceutically pure means at least about 90% pure.
Porcine—of or resembling pigs or swine.
Promoter—A minimal sequence sufficient to direct transcription. Also included in the invention are those promoter elements which are sufficient to render promoter-dependent gene expression controllable for cell type-specific, tissue-specific, temporal-specific, or inducible by external signals or agents; such elements may be located in the 5′ or 3′ or intron sequence regions of the native gene.
Protein—as used herein is intended to include glycoproteins, as well as proteins having other additions. This also includes fragmentary or truncated polypeptides that retain physiological function.
Recombinant—refers to a nucleic acid sequence which is not naturally occurring, or is made by the artificial combination of two otherwise separated segments of sequence. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functional polypeptide sequences to generate a single genetic entity comprising a desired combination of functions not found in the common natural forms. Restriction enzyme recognition sites are often the target of such artificial manipulations, but other site specific targets, e.g., promoters, DNA replication sites, regulation sequences, control sequences, or other useful features may be incorporated by design. A similar concept is intended for a recombinant, e.g., a obesity related transgenic protein according to the instant invention.
Therapeutically-effective amount—An amount of a therapeutic molecule or a fragment thereof that, when administered to a patient, inhibits or stimulates a biological activity modulated by that molecule.
Transformation, “Transfection,” or “Transduction”—Any method for introducing foreign molecules into a cell. Lipofection, DEAE-dextran-mediated transfection, microinjection, nuclear transfer (see, e.g., Campbell et al. BIOL. REPROD. 49:933-942, 1993; Campbell et al., NATURE 385:810-813, 1996), protoplast transgenic, calcium phosphate precipitation, transduction (e.g., bacteriophage, adenoviral retroviral, or other viral delivery), electroporation, and biolistic transformation are just a few of the methods known to those skilled in the art which may be used.
Transformed cell or Transfected cell—A cell (or a descendent of a cell) into which a nucleic acid molecule encoding obesity related has been introduced by means of recombinant DNA techniques. The nucleic acid molecule may be stably incorporated into the host chromosome, or may be maintained episomally.
Transgene—Any piece of a nucleic acid molecule that is inserted by artifice into a cell, or an ancestor thereof, and becomes part of the genome of the animal which develops from that cell. Such a transgene may include a gene which is partly or entirely exogenous (i.e., foreign) to the transgenic animal, or may represent a gene having identity to an endogenous gene of the animal.
Transgenic—Any cell that includes a nucleic acid molecule that has been inserted by artifice into a cell, or an ancestor thereof, and becomes part of the genome of the animal which develops from that cell.
Transgenic Organism—An organism into which genetic material from another organism has been experimentally transferred, so that the host acquires the genetic information of the transferred genes in its chromosomes in addition to that already in its genetic complement.
Ungulate—of or relating to a hoofed typically herbivorous quadruped mammal, including, without limitation, sheep, swine, goats, cattle and horses.
Vector—As used herein means a plasmid, a phage DNA, or other DNA sequence that (1) is able to replicate in a host cell, (2) is able to transform a host cell, and (3) contains a marker suitable for identifying transformed cells.

According to the present invention, there is provided a method for the production of a transgenic protein of interest, the process comprising expressing in the milk of a transgenic non-human placental mammal a transgenic protein useful in the treatment of obesity or related pathologies. The term “treating”, “treat” or “treatment” as used herein includes preventative (e.g., prophylactic) and palliative treatment.

Generation of the Gene Construct

A mammary gland-specific transgene was constructed by inserting the human Antithrombin III (rhAT) cDNA into the caprine beta casein gene (CSN2). The caprine beta casein gene was cloned as an 18.5 Kb fragment in a lambda EMBL3 vector (Roberts, et al., GENE., (1992). 121:255-62). The 6.2 Kb promoter (including exon 1 and part of exon 2) was fused to the rhAT cDNA to direct high level mammary gland-specific expression. A 7.2 Kb 3′ flanking region (including part of exon 7, exon 8, and exon 9) was added to the 3′ end of the rhAT cDNA to help stabilize the expression levels. The 14.95 Kb transgene was excised from bacterial sequences and injected into goat embryos for the production of rhAT in goats' milk.

Identification of Gene Coding for the Protein of Interest

The rhAT cDNA was received from Dr G. Zettlmeissl on the pBAT6 plasmid. The sequence of the cDNA is the same as that published by Bock, et al., NUCLEIC ACIDS RESEARCH, ((1982)) 10: 8113-25), except for the silent nucleotide changes at bp 1096 (T-C) and bp 1417 (A-G).

Identification of Regulatory Sequences of Interest

To direct high level tissue-specific expression of rhAT to the mammary gland of transgenic goats, the goat beta casein gene was cloned from a lambda EMBL3 goat genomic library. The goat beta casein gene is a mammary gland-specific gene which directs expression of high levels of beta casein into the milk. In goats, beta casein is thought to comprise 25-50% of the total milk proteins (about 10-20 mg/ml). The goat beta casein gene was cloned from a Saanen goat genomic library and characterized in transgenic mice.

Goat Species and Breeds

The transgenic goats produced for AT III production are of Swiss origin, and are the Alpine, Saanen, and Toggenburg breeds.

Evaluation of Expression Levels

The expression level of rhAT in the milk of transgenic animals is determined using a thrombin inhibition assay, which measures the inhibition of thrombin's ability to remove a small peptide from an artificial substrate (S2238, Kabi, Franklin Ohio). The basis for this assay is described as follows. The interaction between AT III and thrombin amounts to rapid irreversible inhibition of the protease by AT III in the presence of heparin. However, the interaction is very slow in the absence of heparin. Attempts to extend the range of AT III detectable on a single standard curve reveal that AT III can only be determined accurately in stoichiometric titration across the linear range of standard curves. At low total thrombin concentration (0.7.times.10.sup.-9 M), the effective measuring range for AT III is 0.15-0.75×10⁻⁹ M (e.g., about.7.3-36.8 ng/ml). At high total thrombin, the effective measuring range for AT III is 0.25 to 1.25×10⁻⁹ M (e.g., about 12-60 ng/ml) if the data are fit with a first degree polynomial, and 0.25 to 2.5×10⁻⁹ M (.about.12-120 ng/ml) if the data are fit with a second degree polynomial.

Transgenic rhAT contains a significant amount of oligomannose type and hybrid forms at Asn₁₅₅ and only a very low level of hybrid structures at the other locations. Oligomannose type structures are more primitive structures that are remodeled into the complex type oligosaccharides in the endoplasmic reticulum. Oligomannose structures display masses ranging from Hex5 to HexNAc2, Hex9. These values agree with structures comprised of 5 up to GlcNAc2, Man9, with only the number of mannose residues varying. Hybrid oligosaccharides contain elements of complex oligosaccharides on one antenna of an individual glycosylation site and components of oligomannose type oligosaccharides on the other antenna. The N-linked glycosylation for rhAT was much more heterogeneous than prhAT (plasma derived human antithrombin “prhAT”) with a higher degree of fucosylation and more varied sialylation. Several glycoforms of the molecule with a mass difference of 41 were observed by LC/MS which can be accounted for by the substitution of a Hexose residue by a HexNAc. In view of the monosaccharide composition and the lack of O-linked glycosylation (based on comparison of the observed vs theoretical mass for all peptides other than those containing an N-linked site) this could be accounted for by the substitution of one or more galactose residues by GaINAc.

Several glycoforms with mass differences of 16 mass units were also identified. The difference is explained by the presence of an oxidized form of sialic acid, N-Glycolyineuraminic acid (NGNA) in place of N-Acetyineuraminic acid (NANA). NGNA is a common form of sialic acid found in goats. Approximately 25% of the sialic acids found in rhAT are NGNA. Approximately 25% of goat plasma AT III sialic acid is NGNA.

Thus, we have determined that; (1) one of the four glycosylation sites on rhAT has mainly high mannose (oligomannose) and hybrid type oligosaccharide structures, whereas the prhAT has biantenarry, complex oligosaccharides on each of the four sites; (2) the complex oligosaccharides of rhAT are not fully sialylated, whereas the prhAT oligosaccharides are fully sialylated; (3) the rhAT has a percentage of its sialic acid that is NGNA whereas the prhAT has only NANA; and (4) rhAT contains N-acetylgalactosamine on its N-linked oligosaccharides and the prhAT does not; and (5) the rhAT has fucose on its proximal GlcNAc on each of the three sites having complex oligosaccharides, whereas the prhAT has only a very small amount of fucose on any site.

The rhAT exhibits a faster clearance time in rabbits, mice and monkeys than does prhAT. Twenty μg samples of test AT III was injected via the tail vein and residual AT III determined using an ELISA assay which has little cross reactivity with mouse AT III. The pattern for clearance in mice mimics the pattern found for the same materials in rabbits. The clearance appears to be bimodal and is approximately 10 times faster than for rhAT. In vivo clearance was also examined in a monkey model system. Both trace and high levels of radioiodinated AT III were injected and detected in plasma samples by counting in a gamma counter. The clearance pattern of rhAT in monkeys indicated only a 4 to 5-fold faster clearance from the circulation than the prhAT and could also be defined by a biphasic mechanism.

Early experiments indicate that the rhAT may have a stronger affinity for heparin than the prhAT. This would be important since AT III inhibits thrombin at inflamation or injury sites by binding to heparan sulfate in the endothelial layer of the vasculature. Once bound its affinity for thrombin is enhanced 1000-fold and it binds to and irreversibly inhibits thrombin.

It is known that human AT III is a glycoprotein of a molecular weight of approximately 60 kd which is mainly synthesized in the liver and contained in normal plasma at a concentration of about 150 μg/ml and that human AT III inhibits serine proteases participating in coagulation and fibrinolysis systems including thrombin and factor Xa. The primary structure of human AT III has been clarified by the direct determination of its amino acid sequence (see, in Petersen, T. E. et al., THE PHYSIOLOGICAL INHIBITORS OF BLOOD COAGULATION AND FIBRINOLYSIS, ((Elsevier Science Publishers, Amsterdam,) p. 43 et seq., (1979)) and cDNA cloning (see, Bock, S. C. et al., NUCL. ACIDS RES., 10:8113 (1982); and, Prochownik, E. V. et al., J. BIOL. CHEM., 258:8389 (1982)). According to these reports, human AT III is a single-chain glycoprotein consisting of 432 amino acids which is secreted and formed by excising a signal peptide of 32 residues from a precursor protein. It has four N-linked glycosylation sites in the molecule. The carbohydrate content is about 15% of the molecular weight.

It is an object of the present invention to provide novel human rhAT mutants having a high antithrombin activity useful for the treatment of neurocognitive disorders caused or initiated by major surgical events. It is another object of the present invention to provide a method for mass producing said human rhAT mutants by the recombinant DNA technology, most preferably through transgenic technology.

Isolation of cDNA Coding for rhAT

Since AT III is mainly synthesized in the liver, a commercially available human liver cDNA library may be used for the isolation of cDNA coding for rhAT. Cloning can be effected by a publicly known method. For example, the plaque hybridization method with the use of a synthetic oligonucleotide corresponding to AT III amino acid sequence as a probe (see, Sambrook, J. et al., MOLECULAR CLONING, (Cold Spring Harbor Laboratory (1989)) may be used therefor.

The clones thus obtained are subcloned into a plasmid such as pUC 18, if required. The nucleotide sequence of cDNA thus obtained can be determined and estimated by the Maxam-Gilbert method (see, Maxam, A. M. and Gilbert, W., PROC. NATL. ACAD. SCI. USA, 74:560 (1977)) or the dideoxy method (Sanger, F., SCIENCE, 214:1205 (1981)). The nucleotide sequence of the coding region of AT III cDNA is thus obtained and the amino acid sequence is deduced therefrom.

To recombinantly produce a protein of interest a nucleic acid encoding a transgenic protein can be introduced into a host cell, e.g., a cell of a primary or immortalized cell line. The recombinant cells can be used to produce the transgenic protein, including a cell surface receptor that can be secreted from a mammary epithelial cell. A nucleic acid encoding a transgenic protein can be introduced into a host cell, e.g., by homologous recombination. In most cases, a nucleic acid encoding the transgenic protein of interest is incorporated into a recombinant expression vector.

The nucleotide sequence encoding a transgenic protein can be operatively linked to one or more regulatory sequences, selected on the basis of the host cells to be used for expression. The term “operably linked” means that the sequences encoding the transgenic protein compound are linked to the regulatory sequence(s) in a manner that allows for expression of the transgenic protein. The term “regulatory sequence” refers to promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990), the contents of which are incorporated herein by reference.

Regulatory sequences include those that direct constitutive expression of a nucleotide sequence in many types of host cells, those that direct expression of the nucleotide sequence only in certain host cells (e.g., tissue-specific regulatory sequences) and those that direct expression in a regulatable manner (e.g., only in the presence of an inducing agent). It will be appreciated by those skilled in the art that the design of the expression vector may depend on such factors as the choice of the host cell to be transformed, the level of expression of transgenic protein desired, and the like. The transgenic protein expression vectors can be introduced into host cells to thereby produce transgenic proteins encoded by nucleic acids.

Recombinant expression vectors can be designed for expression of transgenic proteins in prokaryotic or eukaryotic cells. For example, transgenic proteins can be expressed in bacterial cells such as E. coli, insect cells (e.g., in the baculovirus expression system), yeast cells or mammalian cells. Some suitable host cells are discussed further in Goeddel, GENE EXPRESSION TECHNOLOGY: METHODS IN ENZYMOLOGY 185, Academic Press, San Diego, Calif. (1990). Examples of vectors for expression in yeast S. cerevisiae include pYepSecl (Baldari et al., (1987) EMBO J. 6:229-234), pMFa (Kurjan and Herskowitz, (1982) Cell 3:933-943), pJRY88 (Schultz et al., (1987) Gene 54:113-123), and pYES2 (Invitrogen Corporation, San Diego, Calif.). Baculovirus vectors available for expression of transgenic proteins in cultured insect cells include: the pAc series (Smith et al., (1983) MOL. CELL. BIOL. 3:2156-2165) and the pVL series (Lucklow, V. A., and Summers, M. D., (1989) VIROLOGY 170:31-39).

Examples of mammalian expression vectors include pCDM8 (Seed et al., (1987) NATURE 3:840) and pMT2PC (Kaufman et al. (1987), EMBO J. 6:187-195). When used in mammalian cells, the expression vector's control functions are often provided by viral regulatory elements. For example, commonly used promoters are derived from polyoma, Adenovirus 2, cytomegalovirus and SV40.

In addition to the regulatory control sequences discussed above, the recombinant expression vector can contain additional nucleotide sequences. For example, the recombinant expression vector may encode a selectable marker gene to identify host cells that have incorporated the vector. Moreover, to facilitate secretion of the transgenic protein from a host cell, in particular mammalian host cells, the recombinant expression vector can encode a signal sequence operatively linked to sequences encoding the amino-terminus of the transgenic protein such that upon expression, the transgenic protein is synthesized with the signal sequence fused to its amino terminus. This signal sequence directs the transgenic protein into the secretory pathway of the cell and is then cleaved, allowing for release of the mature transgenic protein (i.e., the transgenic protein without the signal sequence) from the host cell. Use of a signal sequence to facilitate secretion of proteins or peptides from mammalian host cells is known in the art.

Vector DNA can be introduced into prokaryotic or eukaryotic cells via conventional transformation or transfection techniques. As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, electroporation, microinjection and viral-mediated transfection. Suitable methods for transforming or transfecting host cells can be found in Sambrook et al. (Molecular Cloning: A Laboratory Manual, 2nd Edition, Cold Spring Harbor Laboratory press (1989)), and other laboratory manuals.

### Methods

This experiments that formed the basis of this specification consisted of multi-center, randomized, double-blind, placebo-controlled studies in which 54 assessable heparin resistant patients undergoing elective cardiac surgery requiring sternotomy and CPB were enrolled in two dosing cohorts. One dosing cohort received a single bolus intravenous injection (75 U/kg) of rhAT, and the other cohort received a single bolus intravenous injection of a normal saline placebo.

Heparin resistance was defined as those patients failing to achieve an ACT of ≧480 seconds after receiving a total dose of 400 U/kg heparin intravenously after anesthesia induction and surgical incision but just prior to CPB. The study was conducted at 11 study centers (8 in the U.S., 2 in Germany, and 1 in The Netherlands). Patients were monitored throughout the study for heparin resistance using ACT II® (the activated clotting time device). Patients were considered treatment failures if fresh frozen plasma (FFP) was required to achieve an ACT of ≧480 seconds after having received their randomized study medication. All patients were closely monitored for adverse events by the study investigator during their hospitalization period and again at the 4-week postoperative follow-up assessment.

Test Product Dose, Mode of Administration and Batch Number:

rhAT:

Supplied in clear, single-dose glass 20-mL vials. Each vial contained a lyophilized cake of 250 mg rhAT. The vial was reconstituted with 10 mL of sterile water for injection (WFI), USP, to provide a 25 mg/mL solution (150 units per mL). Reconstituted rhAT was administered at a dose of 75 U/kg using a dosage/volume/weight table.

Mode of Administration: rhAT administration was achieved by bolus (slow intravenous push) injection.

Batch (Lot) Number: Patients were dosed using a single lot of rhAT during the study: lot # T7010

Efficacy:

The primary efficacy analysis was based on an Intent-to-Treat (ITT) population that included all randomized patients and received the blinded study drug per protocol (n=53). The criterion for the primary analysis was the difference between the proportions of patients requiring FFP in the rhAT group compared to placebo.

Secondary evaluations included the inhibition of thrombin activity as measured by changes in levels of Fibrin Monomer, the inhibition of fibrinolysis as measured by changes in D-Dimer levels, and the changes in plasma AT III activity levels at two time points (30 minutes after initiation of CPB, and preprotamine administration). Differences between the treatment groups in mean values for these parameters were assessed at each time point.

Primary Efficacy Endpoint:

The proportion of rhAT patients (19%) who required administration of two units of fresh frozen plasma (FFP) to achieve an activated clotting time ≧480 seconds was significantly less (p<0.001) than the proportion of placebo patients (81%) who required FFP. This was observed for both the Intent-To-Treat (ITT) and the Per Protocol populations.

Secondary Efficacy Endpoints:

AT III plasma activity levels were significantly higher (p<0.001) in the rhAT treatment group compared with placebo at both the 30 minutes on CPB and at the preprotamine administration time points and for both the ITT and the Per Protocol populations. A cohort analysis (rhAT without FFP versus placebo with FFP) also showed a statistically significant difference in AT III plasma activity level.

The mean change from baseline in the production of D-Dimer, a marker of fibrinolysis and indirectly of thrombin activity, was not statistically significantly different between the treatment groups at the 30 minutes post CPB initiation or the preprotamine time points for the ITT or Per Protocol populations. However, there was an apparent trend toward a smaller increase in D-Dimer levels from baseline at the preprotamine time point among the rhAT patients compared to the placebo patients. A cohort analysis (rhAT without FFP versus placebo with FFP) showed a trend toward smaller increases in D-Dimer levels that approached statistical significance.

Inhibition of fibrin monomer, another secondary efficacy endpoint of the study, is a marker for thrombin activity. There were no significant differences between treatment groups in fibrin monomer levels at any of the time points in either the ITT or the Per Protocol populations. However, fibrin monomer is variably affected by thrombin and fibrinolytic activity, and the assay procedure used was not specific for fibrin monomer. Therefore, changes in the absolute amount of fibrin monomer over time may not have been accurately measured in either treatment group during this study. A cohort analysis (rhAT without FFP versus placebo with FFP) also showed no difference in fibrin monomer level.

Additional exploratory evaluations were performed that further support the efficacy results discussed above.

Safety Results

No adverse event was considered by the study investigators to be definitely or probably related to rhAT administration. Two deaths occurred during the study, both in the rhAT group. Ten rhAT patients and seven placebo patients reported one or more serious adverse events (SAEs) each. Among the adverse events associated with bleeding, hemorrhage NOS was the most frequently reported event in both treatment groups. Mean chest tube drainage volume was significantly greater among patients who received rhAT compared to patients who received placebo during both the 12-hour and the 24-hour postoperative periods. Adverse events associated with bleeding were reported as SAEs for four rhAT patients (3 hemorrhage NOS, 1 coagulation disorder) and for three placebo patients (3 hemorrhage NOS). One SAE (hemorrhage NOS) was considered possibly related to rhAT administration. Additional safety results did not appear to reveal any unexpected safety concerns associated with rhAT treatment, compared with a placebo control.

The results of these experiments support the safety and efficacy of intravenous administration of rhAT (75 U/kg) for the restoration of heparin responsiveness and prevention of neurocognitive dysfunctions realized as a result of surgical procedures.

Abbreviations and Terms

Abn. Abnormal
ACT Activated Clotting Time
Admin. Administration
AE Adverse Event
ALAT (SGPT) Alanine Aminotransferase
ANOVA Analysis of Variance
APPT Activated Partial Thromboplastin Time
ASAT (SGOT) Aspartate Aminotransferase
bpm Beats per minute
BUN Blood Urea Nitrogen
° C. Degrees centigrade
Ca++ Calcium
CABG Coronary Artery Bypass Graft
CFR Code of Federal Regulation
CICU Cardiac Intensive Care Unit
CPB Cardiopulmonary Bypass
CPK Creatinine Phosphokinase
CPMP Committee for Proprietary Medicinal Products
CRF Case Record Form
CT Computerized Tomography
Ct. Count
CV Curriculum Vitae
Dev. Deviation
Dis. Disorders
dL Deciliter
Δ delta (change)
DSMB Data Safety Monitoring Board
DVT Deep Vein Thrombosis
EC European Community
ECG Electrocardiogram
ECOG Eastern Cooperative Oncology Group
ELISA Enzyme-linked Immunosorbant Assay
et al. et alii (and others)
e.g. exampli gratia (for example)
etc. etcetera
FDA Food & Drug Administration
FFP Fresh Frozen Plasma
Func. Function
g Gram(s)
GCP Good Clinical Practice
g % Grams percent
GTC GTC Biotherapeutics Incorporated
H₀ Null Hypothesis
HCT Hematocrit
HDR Heparin Dose Response
Hep Heparin
Hgb Hemoglobin
HIV Human Immunodeficiency Virus
HMS Hemostasis Management System
Hosp. Hospital
Hx History
ICH International Conference on Harmonization
ICU Intensive Care Unit
i.e. id est (that is)
IEC Independent Ethics Committee
Intra-op Intra-operative
Inv. Investigator
IRB Institutional Review Board
ITT Intent-To-Treat
IU International Unit
IV/iv Intravenous
kg Kilogram
L Liter
LDH Lactate Dehydrogenase
LLC Limited Liability Corporation
MA Massachusetts
Max. Maximum
MCH Mean Corpuscular Hemoglobin
MCHC Mean Corpuscular Hemoglobin Concentration
MCV Mean Corpuscular Volume
MD Medical Doctor
mg milligrams
μl microliter
min. Minute(s)
Min. Minimum
ML milliliter
mmHg Millimeters of mercury
N/A Not Applicable
Neutroph. Neutrophil
N Number
n Number
ng Nanogram(s)
nmol nanomole(s)
NMR Nuclear Magnetic Resonance
NOS Not Otherwise Specified
NP Not Provided
NR Not Related
OR Operating Room
Post-op Post-operative
PPSP Factor Substitution
Pre-op Pre-operative
PRBC Packed Red Blood Cells
Pt. Patient
PT Prothrombin Time
PTT Partial Thromboplastin Time
RBC Red Blood Cells
RBC/HPF Red Blood Cells/High Powered Field
rhAT Recombinant Human Antithrombin III
SAE Serious Adverse Event
SAS Statistical Analysis Software
Sec. Second(s)
Seg. Segmented
Sept. September
SICU Surgical Intensive Care Unit
Std. Standard
Stnd. Standard
Surg. Surgery
TIA Transient Ischemic Attack
tPA Tissue Plasminogen Activator
U Units
U/kg Units per kilogram
U.S. United States
USP United States Pharmacopeia
WBC White Blood Cell(s)
WBC/HPF White Blood Cells/High Powered Field
WFI Water for Injection
WHO World Health Organization
WHOART WHO Adverse Reaction Thesaurus
yr Year(s)
> Greater than
< Less than
≧ Greater than or equal to
≦ Less than or equal to

In normal physiological states, the vascular endothelium and multiple circulating factors play important roles in preventing thrombosis. Endothelial factors and circulating plasma proteins instrumental in preventing thrombosis include prostacyclin, endothelium-derived relaxing factor, tissue plasminogen activator, endothelial heparan sulfate, proteins C and S, heparin cofactor III, and antithrombin III (AT III). AT III is a naturally occurring plasma protein that inhibits thrombin (as well as other circulating coagulation factors) and binds to heparan sulfate moieties on the vascular endothelium to help maintain homeostasis of the hemostatic system. However, in non-physiologic states such as cardiopulmonary bypass (CPB), during which blood interfaces with an extracorporeal circuit, a tremendous prothrombotic stimulus is initiated. Potent anticoagulation, achieved primarily with unfractionated intravenous heparin administration, is the most important therapeutic intervention used to facilitate extracorporeal circulatory support and prevent catastrophic thrombotic events.

Heparin attains anticoagulation in the presence of the powerful prothrombotic stimulus of CPB by causing a conformational change in the AT III tertiary structure catalyzing the reaction between AT III and thrombin approximately 1,000 fold higher than in the absence of heparin. Thus, heparin's anticoagulant effect is closely tied to AT III activity. Therefore, it follows that abnormally low AT III plasma activity can lead to an altered anticoagulant response to heparin.

In the setting of CPB, the degree of anticoagulation is usually monitored by the activated clotting time (ACT), a simple and rapidly performed “point of care” test. The decision to initiate CPB after heparin administration hinges on attainment of an adequate ACT reflecting adequate anticoagulation. Limited experimental data exist that define the optimal ACT for initiation of cardiopulmonary bypass (CPB). Young, et al., in a primate study, observed fibrin monomer production in 5 of 6 animals whose initial ACT was less than 400 seconds. Historically, values less than 300 seconds were associated with grossly visible clots in the bypass circuit. In a survey of members of the Society of Cardiovascular Anesthesiology and American Society of Extracorporeal Circulation, Michelson, et al., found that the target ACT used by 82% (816/1000) of responders was ≧400-480 seconds, with an additional 4.5% (45/1000) of responding members targeting a higher ACT.

Heparin resistance can be considered as the failure to achieve the desired ACT after a typical dose of heparin. Failure to achieve an acceptable ACT (reflecting inadequate anticoagulation) for CPB is usually managed by administration of additional heparin. Since the etiology of heparin resistance during CPB is often caused by low plasma AT III activity, clinicians may choose to administer an exogenous source of AT III (e.g., fresh frozen plasma (FFP) or a plasma-derived AT III) to restore heparin sensitivity. The overall incidence of the heparin resistant state is not well defined and will vary as a function of heparin dose and target ACT. For example, George Despotis, MD, from Washington University, determined that 9.9% (13/131) of patients who were not on preoperative heparin infusions and 12.8% (14/109) of patients who had been on preoperative heparin infusions were heparin resistant. In this setting, heparin resistance was defined as a failure to achieve an ACT>480 seconds after 400 U/kg of heparin. Ellise Delphin, MD, from the Department of Anesthesiology, Columbia University, identified 75 out of 1600 (4.7%) patients undergoing CPB considered to be heparin resistant. In this patient population, heparin resistance was defined as the failure to achieve an ACT≧480 seconds after >500 U/kg of heparin, or ≧600 seconds if the patient was on aprotinin. Finally, Williams, et al., found that 3.7% (85/2279) of patients were heparin resistant, as they did not achieve an ACT>480 seconds after 450 U/kg of heparin.

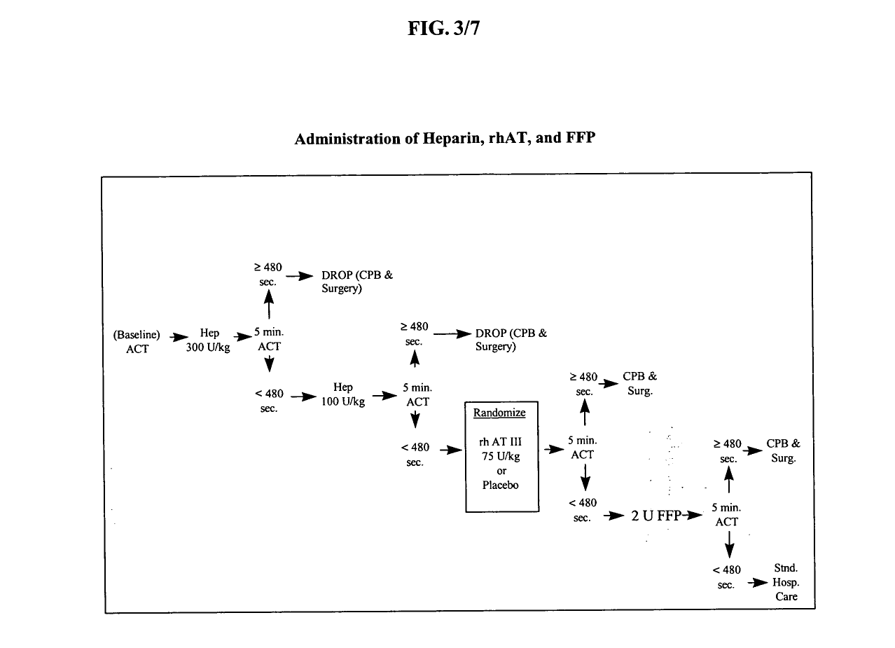

The study endpoint for evaluation of efficacy was to be whether or not the patient met the requirement for administration of FFP. An ACT<480 seconds five minutes after injection of study treatment was to warrant administration of two units of FFP. Patients who met this requirement but did not receive FFP were to be included in the population of patients who received FFP. It was anticipated that most heparin resistant patients who received rhAT would not require FFP administration, while most receiving placebo would require the administration of FFP. The schema that was to be used for heparin dosing, randomization, and FFP administration is provided in FIG. 3.

The choice of a prospective, randomized, double-blind, placebo-controlled study design was intended to minimize the potential for subjective bias. Overall, the study design attempted to capture and isolate from placebo, the effects of rhAT therapy in heparin resistant patients.

The study population for both treatment cohorts was to consist of heparin resistant patients scheduled for elective cardiac surgery requiring sternotomy and cardiopulmonary bypass (CPB). This patient population was chosen to study the safety and efficacy of rhAT because AT III given in the form of fresh frozen plasma is a therapeutic approach sometimes used to facilitate anticoagulation with heparin in heparin resistant patients prior to and during extracorporeal circulation. Patients who are considered to be heparin resistant represent only a small fraction of the potential patient population (approximately 5-10%).

Treatments

There were two treatment cohorts in this study. Approximately 26 patients were to be randomized to receive rhAT as a single intravenous dose of 75 U/kg, and approximately 26 patients were to be randomized to receive a single intravenous dose of normal saline placebo.

Identity of Investigational Product

RhAT, was supplied in clear glass, single-dose 20 mL vials and was refrigerated at 2-8° C. until reconstituted. The The Inventors Clinical Supply Unit located at the Allston Landing Facility in Allston, Mass. supplied investigational product to each study site. Each vial contained a lyophilized cake of 250 mg rhAT. The vial of lyophilized rhAT was reconstituted with 10 mL of sterile water for injection (WFI), USP, to provide a 25 mg/mL solution. Sterile WFI was injected into the vial, being careful to add the liquid along the side of the vial to prevent foaming. The vial was swirled gently (not shaken) to allow adequate dissolution of the product.

The research pharmacist working in the institutional pharmacy department at each study site was to prepare all study materials. The pharmacist, upon notification by the study team, prepared study drug according to the Study Drug Randomization Code. Only the research pharmacist had access to the code. A drug inventory record for monitoring drug accountability was kept, which included the total number of vials shipped and the number of vials remaining.

The volume of reconstituted product drawn for administration to each patient was to be determined using the Study Drug Dosage/Volume/Weight Table (see study protocol). The dose was drawn into a 60 mL syringe, after which the pharmacist added normal saline such that the total volume was equal to 60 mL. Only one production batch of rhAT, Lot # T7010, was used in this study.

Selection of Doses in the Study

The rhAT dose, 75 U/kg, administered to patients in this study was based on results obtained in a previous pharmacology study, entitled “A Phase I-II Open, Dose Escalation Study of the Safety of Recombinant Human (transgenic) Antithrombin III in Patients Scheduled for Primary Cardiac Surgery Requiring Cardiopulmonary Bypass.” In this study of patients whose endogenous AT III was depleted due to preoperative heparin-induced consumption, it was established that dosing with ≧50 U/kg of rhAT achieved plasma AT III levels that were approximately 100% of normal (baseline) and were maintained for the duration of CPB. In addition, at rhAT doses up to 200 U/kg where plasma AT III levels reached 600% of normal, no adverse effects were observed. In view of these data, selection of the 75 U/kg was felt to be adequate to achieve the desired efficacy goal of the current study while posing little, if any, safety concern.

Selection and Timing of Dose for Each Patient

The 52 patients were to be randomly assigned to receive either rhAT or normal saline placebo from a randomization schedule generated by The Inventors Corporation's Biometrics Department. Twenty-seven patients were randomized to the rhAT study group, and 27 patients were randomized to the placebo study group, totals that exceeded the target number of patients for each treatment group.

The timing of dosing (time of day) of study medication and the relationship of dosing to meals was not specified in the study protocol. The relationship of dosing to meals was not an issue in view of intravenous administration of study medication. The study population was to consist of patients scheduled for cardiac surgery requiring CPB. The timing of food consumption and the timing of dosing of study medication were to be determined according to standard preoperative hospital procedures and the surgical schedule of the hospital.

Blinding

This was a double-blind, randomized, and controlled trial. Neither the patient nor the investigator knew which treatment was administered. In addition, key clinical, medical, regulatory, and biostatistical personnel of the Sponsor were to remain blinded throughout the study. The blind was not to be broken for the purpose of data analysis until all decisions regarding the evaluability of patients had been made and the database had been finalized.

A blinded randomization schedule was generated for the study sites. Each study site (institutional pharmacist) was to be given an initial assignment for 8 patients. Additional assignment sets were to be given to sites that enrolled 8 patients prior to achieving the study target enrollment of 52 patients.

The institutional pharmacist at each study site performed randomization according to the predetermined blinded randomization schedule. The pharmacist was the only person who had access to the blinded randomization schedule and was responsible for keeping the integrity of the blind and for preparing the blinded study drug. The pharmacist was to keep a drug inventory record that included the total number of vials shipped and the number of vials remaining.

The blind for this study was not to be broken unless the patient experienced a Serious Adverse Event for which, in the opinion of the attending physician, unblinding was deemed absolutely necessary for the patient's treatment. In this situation, the principal or sub-investigator was to first attempt (if time allowed) to call the appointed pharmacovigilance contact at The Inventors to obtain permission to break the code.

Prior and Concomitant Therapy

Patients were not to receive any antifibrinolytic agent [i.e., aprotinin, tranexamic acid, epsilon aminocaproic acid (Amicar®)] within 24 hours prior to administration of the blinded study drug or prior to the preprotamine blood draw at the end of bypass. Patients were excluded from the study if they had recently received or were receiving any of the following medications at the time of randomization: warfarin (within three days); streptokinase (within 48 hours); tPA (within 48 hours); or abciximab (Reopro® (within one week)).

Allowed concomitant therapy was not expected to affect the outcome of the study; however, such effects could not be entirely discounted. At study initiation there were no known drug interactions or direct effects of any concomitant medications, other than those excluded in the protocol, with rhAT.

Any new medications or medications that had their dose or frequency changed during the hospitalization period of the study were to be recorded on the patient's case record form (CRF).

Efficacy Measurements

Treatment Period

The following blood/plasma samples were obtained for efficacy/bioactivity parameters just prior to study drug administration (“0 min.”), at 30 minutes after the initiation of CPB, and just prior to heparin reversal with protamine:
Kaolin ACT without heparinase
Fibrin Monomer to assess thrombin activity
D-Dimer to assess inhibition of fibrinolysis
AT III levels

Medtronic's Hepcon® Hemostasis Management System (HMS) for measuring the heparin dose response was used to aid in patient evaluation for possible heparin resistance and to standardize this multi-centered study as much as possible. The ACT II® (Automated Coagulation Timer) device was used for measuring the activated clotting time during the treatment period. ACTrac®, an electronic calibration device, was to be used in conjunction with the ACT II® device to insure that evaluation values using Hepcon® and study values using ACT II® were consistent between the two devices. Additionally, a calibration log was to be kept at each study site.

These devices were used in conjunction with the following cartridges:
Heparin Dose Response Cartridge to measure the patients' sensitivity to heparin
Kaolin ACT cartridge to measure Activated Clotting Times
Heparin Protamine Titration cartridge to measure whole blood heparin concentration during CPB
   Heparin Loading Doses and Study Drug Administration

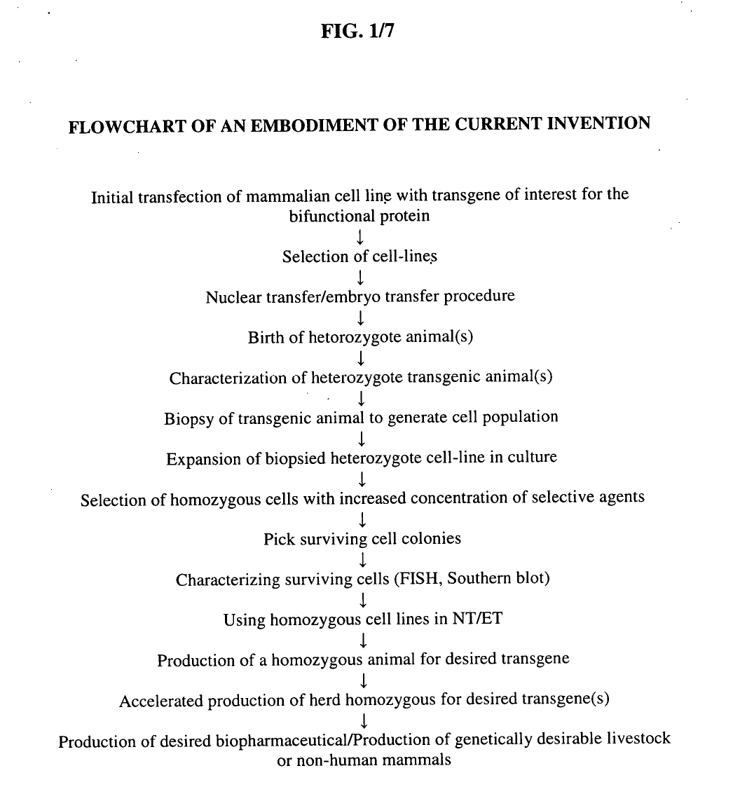

Anesthesia induction and surgical incision were performed per institutional protocol. When bypass was ready to commence, the patient was heparinized as described below and outlined herein in FIG. 1. Patients were considered “enrolled” into the study at this point. Data collected for “enrolled” patients included patient initials, demographics, heparin doses, and resulting ACT responses.

Those patients meeting the final inclusion criterion as defined by an ACT<480 seconds after a total heparin loading-dose of 400 U/kg were randomized. Only patients randomized to treatment were followed for safety and efficacy/bioactivity parameters as per protocol.

Baseline ACT: An ACT was obtained prior to heparin administration.

First Heparin Loading Dose:

Porcine Heparin (300 U/kg) was administered intravenously and 5 minutes later, the ACT was determined.

If the ACT was ≧480 seconds, the patient was withdrawn from further study participation. Surgery proceeded per institutional protocol.

If the ACT was <480 seconds, a second dose of heparin was administered.

Second Heparin Loading Dose:

The second dose of heparin (100 U/kg) was administered intravenously and 5 minutes later, the ACT was determined.

If the ACT was ≧480 seconds, the patient was withdrawn from further study participation. Surgery proceeded per institutional protocol.

If the ACT was <480 seconds, the patient was randomized to receive one of two intravenous study drug treatments (placebo or 75 U/kg rhAT).

Study Drug Administration

All patients receiving blinded study drug were considered as “Treated & Randomized” and were to complete all study requirements. Vital signs and all safety and efficacy/bioactivity laboratory studies were obtained prior to study drug administration. Blinded study drug, either rhAT or placebo, was administered by bolus (slow intravenous push). Vital signs were taken one minute after administration of study medication. Vital signs were to be taken again at 5 minutes following study drug administration in addition to determination of the ACT.
If the ACT was ≧480 seconds, aortic cannulation and CPB commenced.
If the ACT was <480 seconds, patient was to receive 2 units of FFP.

Vital signs were obtained again at 10 minutes post study drug administration.

Post FFP Administration: The ACT was determined 5 minutes after FFP administration.
If the ACT was ≧480 seconds, aortic cannulation and CPB commenced.
If the ACT was <480 seconds, the patient was treated according to standard hospital practice.

Surgery

The surgical procedure was to be performed according to the standard of care at each of the treating institutions. Bioactivity laboratory studies and the ACT were obtained at “30 minutes on CPB” and “preprotamine” time points (see Table 1 Study Flow Chart). All peri-operative blood products administered were to be recorded up to the time of discharge from the hospital.

Heparin Maintenance

During bypass additional heparin was administered as needed to maintain whole blood heparin concentrations at 3.4 U/mL, as guided by the automated protamine titration method (Hepcon®) and ACT values. In hospitals that had an institutional protocol for intra-operative heparin maintenance, the method for heparin maintenance described above was waived, and additional heparin was administered during the bypass according to institutional standard practice.

Heparin Reversal

Heparin reversal with protamine was to be per institutional protocol. The time and dose of all protamine administrations were recorded.

Post-Operative Period

Chest tube drainage was recorded until the tube was removed. Safety laboratory studies and ECG were obtained 24-48 hours post-operatively for all “treated and randomized” patients. Four weeks post-operatively, a blood sample for rhAT antibodies and viral studies was to be obtained, in addition to a post-operative review to identify any Serious Adverse Events that may have occurred during the four-week period.

Primary Efficacy Variables

The primary efficacy endpoint of the study was a comparison of the difference between rhAT and placebo in the avoidance of the use of fresh frozen plasma (FFP) to achieve an ACT≧480 seconds in heparin resistant patients. This was accomplished by comparing the proportion of patients requiring FFP in the two treatment groups (rhAT versus placebo). All patients who warranted FFP administration based on the protocol requirements (failure to achieve an ACT of ≧480 seconds as measured 5 minutes after administration of blinded study medication) were considered to have received FFP whether or not it was actually administered. Specifically, patients who failed to complete the study after being randomized (i.e., who were unable to have an ACT assessment subsequent to receiving blinded study drug or who failed to receive FFP despite meeting the requirements for FFP) were included in the group of patients who received FFP.

Secondary Efficacy Endpoints

Secondary efficacy endpoints were to compare the following laboratory parameters on an Intent-To-Treat basis in patients randomized to rhAT versus placebo:
The inhibition of thrombin activity (as assessed by change in Fibrin Monomer plasma levels)
The inhibition of fibrin formation and fibrinolysis (as assessed by changes in D-Dimer plasma levels)
The change in plasma AT III activity level

Blood/plasma samples were obtained just prior to study drug administration, at 30 minutes after the initiation of CPB, and just prior to heparin reversal with protamine for determination of the secondary efficacy endpoints (see Table 1—Flow Chart).

Statistical Methods Planned in the Protocol and Determination of Sample Size

Efficacy Analysis

To assess the comparability of treatment groups, clinical and demographic variables at baseline are to be presented using descriptive statistics. The primary efficacy analysis is to be based on an Intent-To-Treat population (ITT), which was to include all randomized patients. The primary efficacy analysis—the difference between the proportion of patients requiring FFP in the rhAT group compared to placebo—is to be assessed using Pearson Chi-Square test or Barnard's Unconditional Exact test, depending on the data distribution. Patients who do not complete the study, including, but not limited to, those who are not evaluated for an ACT after receiving blinded study drug, or who fail to achieve an ACT of ≧480 seconds after receiving blinded study drug but do not receive FFP, are to be included in the ITT population and considered to have received FFP for the primary efficacy analysis. In the event that important demographic or prognostic variables (i.e., age, weight, or surgical procedure) differ between the groups, a secondary analysis may be performed. Differences in the treatment effect on the odds of requiring FFP will be assessed using logistic regression. Odds ratios, comparing rhAT to placebo, and 95% confidence intervals will be presented.

The secondary efficacy endpoints—levels of Fibrin Monomer, D-Dimer, and plasma AT III activity will be examined at two time points: 30 minutes after initiation of CPB and preprotamine administration. Differences between the group means will be assessed at each study time point using an analysis of variance (ANOVA) including treatment and study center as main effects. Should distribution requirements not be satisfied, non-parametric approaches may be used. A subset analysis may also be performed to compare the patients receiving rhAT only and those receiving FFP only.

Determination of Sample Size

An estimate of 24 assessable patients in each treatment group would provide 80% power to detect a 40% absolute reduction (65% versus 25%) in the proportion of patients requiring fresh frozen plasma (FFP). To account for a dropout rate of approximately 10%, a total of 52 randomized patients, 26 in each treatment group, were recommended for the trial. Table 2 provides the number of patients that would be required per treatment group at various proportions of patients in each treatment group requiring FFP (two-sided, α=0.05).

TABLE 2
Estimated Sample Size Requirements

| | |
|---|---|
| Failure Rate (%) | Number (n) of Patients/Group |

| Rh AT III | Placebo | 80% Power | 90% Power |
|---|---|---|---|
| 20 | 65 | 18 | 24 |
| 20 | 70 | 15 | 19 |
| 25 | 65 | 24 | 31 |
| 25 | 70 | 19 | 24 |

Changes in the Conduct of the Study or Planned Analysis

Five amendments to the protocol (approved Jan. 28, 1998) were made after the study was initiated in May 1998. The effective dates for the five amendments were as follow: Sep. 10, 1998 (Amendment 1); Oct. 28, 1998 (Amendment 2); Jan. 18, 1999 (Amendment 3); May 26, 1999 (Amendment 4); and Jul. 29, 1999 (Amendment 5). Seven, 15, 18, 22, and 24 patients had been screened and signed informed consent forms prior to implementation of the first, second, third, fourth, and fifth amendments, respectively. None of the changes in any of the five amendments was felt to have an impact on the interpretation of the study data.

Analysis of Efficacy

Primary Efficacy Endpoint and Disposition of Patients

A flow chart summarizing patient recruitment beginning with all patients screened is provided in Table 3 below.

TABLE 3
Patient Recruitment Flow Chart

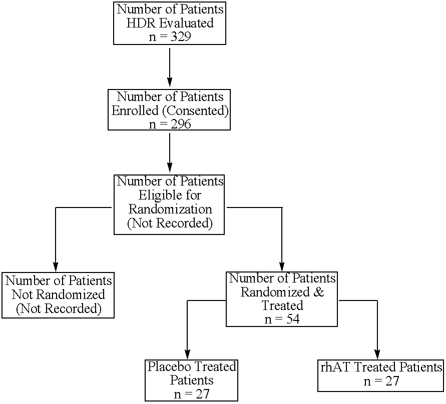

A total of 329 patients were HDR screened for entry into the study. The number of patients found to have met all eligibility criteria except for the final randomization criteria (determined in the operating room) was not recorded. In addition, the total number of patients that met the randomization criteria was not recorded but of those that did, 54 patients were randomized into the study. Reasons for why the other eligible patients were not randomized cannot be provided. Twenty-seven of the patients were randomized into the placebo treatment group and 27 were randomized into the rhAT treatment group. All placebo patients completed the trial (n=27), while 24 of the rhAT patients completed the study. One rhAT patient (Patient 02-035) received only a partial dose of study drug (1 mL) after which administration was stopped in view of the surgeon deciding to perform the surgery “off-pump”. Two other rhAT patients (Patients 04-024 and 13-197) died, 2 an 18 days, respectively, post-operatively due to complications associated with their underlying disease. The study blind was broken for one patient prior to study completion and finalization of the database; the code for Patient 13-197 in the rhAT treatment group was unblinded for the DSMB.

The disposition of study patients is summarized by study phase in Table 4 below.

TABLE 4
Patient Disposition

| | | Number of Patients | |
|---|---|---|---|
| Study Phase | Statistic | Placebo | rhAT |
| Randomized and Received Treatment | n | 27 | 27 |
| Pre-Bypass | n | 27 | 27 |
| Study Drug Administered | n | 27 | 26* |
| Postoperative - Hospitalization | n | 27 | 26 |
| Postoperative - 4-Week Follow-up | n | 27 | 24** |

*rhAT patient 02-035 received a partial dose (1 mL) of study medication
**rhAT patients 04-024 and 13-197 died post-operatively.

Patient recruitment into each treatment group by study site is provided in Table 4.

Study site 12 contributed the most patients (n=10) to the study population. Importantly, across all of the study centers, the distribution of patients randomized to receive rhAT or placebo was well balanced.

The primary efficacy endpoint of the study was the comparison of the proportion of patients in each treatment group requiring the infusion of fresh frozen plasma (FFP) after administration of study medication but prior to initiation of CPB. Analysis of the primary endpoint was performed on the Intent-To-Treat (ITT) and the Per Protocol populations. Table 5 summarizes the proportion of patients in each treatment group who received FFP, as observed in each of the two patient populations.

TABLE 5
Primary Efficacy Results: Patients' FFP Administration
Status (Intent-To-Treat and Per Protocol Populations)

| | Treatment Group | | | | |
|---|---|---|---|---|---|
| | Placebo | | rhAT | | |
| Study | | Number FFP | | Number FFP | |
| Population | n | Treated (%) | n | Treated (%) | P-Value |
| Intent-To- | 27 | 22 (81) | 26 | 5 (19) | <0.001 |
| Treat | | | | | |
| Per Protocol | 24 | 19 (79) | 21 | 3 (14) | <0.001 |

FFP = Fresh Frozen Plasma (2 units)
P-values based on Barnard's Unconditional Exact test for difference between two binomial proportions.
Note:
The percentages are based on the total number of patients in each treatment group.
Note:
Patients requiring FFP are defined as all patients failing to achieve an ACT ≧480 seconds 5 minutes after administration of study medication regardless of the actual status of FFP administration.
Note:
Patients 01-069 (rhAT) and 01-071 (placebo) did not have ACTs taken and were considered to have received FFP.

In the Intent-To-Treat population, 22 of 27 placebo treated patients required the infusion of FFP, while only five of 26 rhAT treated patients required FFP prior to initiation of CPB. The proportion of patients requiring the administration of two units of FFP prior to proceeding to CPB was significantly smaller (p<0.001; Barnard's Unconditional Exact Test) in the rhAT treatment group (19%) compared to the proportion among placebo treated patients (81%). The same level of statistical significance was observed when the analysis was performed using the Per Protocol population.

The distribution of the primary efficacy endpoint outcomes across study centers was reviewed for both treatment groups. These data are displayed in Table 6.

TABLE 6
Outcome (FFP or No FFP) Distribution
by Study Center (Intent-To-Treat Population)

| Treatment Group and Outcome | |
|---|---|
| Placebo (n = 27) | rhAT (n = 26) |

| Study Center | FFP | No FFP | FFP | No FFP |
|---|---|---|---|---|
| 01 | 3* | 2 | 1* | 2 |
| 02 | 1 | 0 | 0 | 1 |
| 03 | 1 | 0 | 0 | 2 |
| 04 | 4 | 0 | 0 | 4 |
| 05 | 1 | 0 | 0 | 0 |
| 06 | 1 | 0 | 0 | 2 |
| 08 | 1 | 0 | 0 | 2 |
| 09 | 0 | 1 | 0 | 0 |
| 10 | 3 | 1 | 0 | 3 |
| 12 | 4 | 1 | 3 | 2 |
| 13 | 3 | 0 | 0 | 4 |
| Total | 22 | 5 | 4 | 22 |

FFP = Fresh Frozen Plasma
*Patients 01-069 (rhAT) and 01-071 (placebo) did not have ACTs taken and were considered to have received FFP.

Although no formal statistical comparisons were made, the primary efficacy outcomes appear to be similarly distributed across study centers for both treatment groups. In no case was a single center responsible for a particular outcome in either treatment group.

Secondary Efficacy Endpoints

The secondary efficacy endpoints were measures of the comparative effects of each treatment on laboratory measures that could potentially be affected by rhAT or FFP administration. These “bioactivity” measures included fibrin monomer, D-Dimer, and AT III blood plasma activity levels taken just prior to and 30 minutes after administration of study medication and just prior to heparin reversal with protamine at the end of CPB.

TABLE 7
D-Dimer: Actual Value Descriptive Statistics
(nmol/L) (Intent-To-Treat Population)

| | | | 30 Minute Post | | | |
|---|---|---|---|---|---|---|
| | Baseline | | Initiation of CPB | | Preprotamine | |
| Statistic | rhAT | Placebo | rhAT | Placebo | rhAT | Placebo |
| N | 26 | 26 | 25 | 26 | 25 | 26 |
| Mean | 153.2 | 223.5 | 721.8 | 643.8 | 1606.3 | 1663.2 |
| Std. Dev. | 192.7 | 329.8 | 1016.2 | 943.6 | 1944.4 | 1390.0 |
| Minimum | 6 | 10 | 51 | 18 | 67 | 6 |
| Maximum | 782 | 1499 | 4356 | 4924 | 7097 | 6062 |

| | | | |
|---|---|---|---|
| P-Value | 0.641 | 0.785 | 0.224 |
| for Group | | | |
| Differences | | | |

Std. Dev. = Standard Deviation
Note:
Subject counts may vary due to missed sampling or samples that could not be assayed.
Note:
The normal range for D-Dimer is less than or equal to 100 ng/mL.

For the Intent-To-Treat population, there were no statistically significant differences in mean D-Dimer levels between the two treatment groups at baseline, 30 minutes after initiation of CPB, or prior to protamine administration. The mean change from baseline to 30 minutes post initiation of CPB and the mean change from baseline to the preprotamine administration time point for D-Dimer is provided in Table 8 for both treatment groups.

TABLE 8
D-Dimer Mean Change from Baseline
(nmoles/L) (Intent-To-Treat Population)

| | 30 Minutes Post CPB | | Preprotamine Administration | |
|---|---|---|---|---|
| Statistic | rhAT | Placebo | rhAT | Placebo |
| N | 25 | 26 | 25 | 26 |
| Mean | 564.6 | 420.3 | 1457.5 | 1439.7 |
| Std. Dev. | 961.9 | 904.5 | 1914.6 | 1294.0 |
| Minimum | −83 | −173 | 29 | −76 |
| Maximum | 3994 | 4561 | 6863 | 5699 |

| | | |
|---|---|---|
| P-Value | 0.799 | 0.197 |
| for Group | | |
| Differences | | |

Std. Dev. = Standard Deviation
Note:
Subject counts may vary due to missed sampling or samples that could not be assayed.
Note:
The normal range for D-Dimer is less than or equal to 100 ng/mL.
Note:
Subject 04-023's preprotamine measure has been excluded.

For the Intent-To-Treat population, there were no statistically significant differences in mean change in D-Dimer levels between the two treatment groups at 30 minutes after initiation of CPB or prior to protamine administration.

Fibrin Monomer Level

Table 9 provides the actual value descriptive statistics for fibrin monomer levels at baseline, 30 minutes post initiation of CPB, and at the preprotamine time-point for both treatment groups.

TABLE 9
Fibrin Monomer - Actual Value Descriptive
Statistics (ng/mL) (Intent-To-Treat Population)

| | | | 30 Minute Post | | | |
|---|---|---|---|---|---|---|
| | Baseline | | Initiation of CPB | | Preprotamine | |
| Statistic | rhAT | Placebo | rhAT | Placebo | rhAT | Placebo |
| N | 23 | 24 | 23 | 23 | 21 | 25 |
| Mean | 30.4 | 31.9 | 27.3 | 28.3 | 29.1 | 32.0 |
| Std. Dev. | 13.3 | 11.6 | 12.4 | 9.3 | 12.2 | 10.0 |
| Minimum | 13 | 14 | 12 | 16 | 15 | 16 |
| Maximum | 55 | 56 | 52 | 57 | 54 | 55 |

| | | | |
|---|---|---|---|
| P-Value | 0.687 | 0.773 | 0.381 |
| for Group | | | |
| Differences | | | |

Std. Dev. = Standard Deviation
Note:
Subject counts may vary due to missed sampling or samples that could not be assayed.
Note:
Values reported as < or > have been excluded.
Note:
The normal range for fibrin monomer is less than or equal to 15 nmol/L.

For the Intent-To-Treat population, there were no statistically significant differences in mean fibrin monomer level between the two treatment groups at baseline, 30 minutes after initiation of CPB, or prior to protamine administration. The mean change from baseline to 30 minutes post initiation of CPB and the mean change from baseline to the preprotamine administration time point for fibrin monomer level is provided in Table 10 for both treatment groups.

TABLE 10
Fibrin Monomer Mean Change from Baseline
(ng/mL) (Intent-To-Treat Population)

| | 30 Minutes Post CPB | | Preprotamine Administration | |
|---|---|---|---|---|
| Statistic | rhAT | Placebo | rhAT | Placebo |
| N | 21 | 21 | 19 | 23 |
| Mean | −1.4 | −2.5 | 2.6 | 1.0 |
| Std. Dev. | 8.6 | 8.5 | 6.5 | 9.1 |
| Minimum | −20 | −22 | −11 | −30 |
| Maximum | 14 | 15 | 17 | 22 |

| | | |
|---|---|---|
| P-Value | 0.667 | 0.522 |
| for Group | | |
| Differences | | |

Std. Dev. = Standard Deviation
Note:
Subject counts may vary due to missed sampling or samples that could not be assayed.
Note:
Values reported as < or > have been excluded.
Note:
The normal range for fibrin monomer is less than or equal to 15 nmol/L.

For the Intent-To-Treat population, there were no statistically significant differences in mean change in fibrin monomer level between the two treatment groups at 30 minutes after initiation of CPB or prior to protamine administration.

AT III Plasma Activity Level

Table 11 provides the actual value descriptive statistics for AT III plasma activity level at baseline, 30 minutes post initiation of CPB, and at the preprotamine time-point for both treatment groups.

TABLE 11
AT III Plasma Activity Level - Actual Value
Descriptive Statistics (%) (Intent-To-Treat Population)

| | | | 30 Minute Post | | | |
|---|---|---|---|---|---|---|
| | Baseline | | Initiation of CPB | | Preprotamine | |
| Statistic | rhAT | Placebo | rhAT | Placebo | rhAT | Placebo |
| N | 26 | 26 | 25 | 26 | 25 | 26 |
| Mean | 78.0 | 73.8 | 122.1 | 51.6 | 112.6 | 50.7 |
| Std. Dev. | 23.5 | 15.5 | 18.1 | 11.9 | 30.6 | 13.2 |
| Minimum | 38 | 54 | 83 | 26 | 67 | 21 |
| Maximum | 140 | 116 | 159 | 83 | 204 | 76 |

| | | | |
|---|---|---|---|
| P-Value | 0.451 | <0.001 | <0.001 |
| for Group | | | |
| Differences | | | |

Std. Dev. = Standard Deviation
Note:
Subject counts may vary due to missed sampling or samples that could not be assayed.
Note:
The normal range for plasma AT III activity is 70-130%.

Using the Intent-To-Treat population, there was no statistically significant difference between the rhAT and the placebo treatment groups in the mean plasma AT III activity level at baseline. The mean AT III activity level was above the lower limit of normal physiologic activity (70%) at baseline for patients in both cohorts (78% and 74%, respectively). However, there were statistically significant differences (p<0.001) between the two treatment groups with respect to plasma AT III activity at 30 minutes post CPB initiation and preprotamine reversal. In the rhAT treatment group, the mean plasma AT III activity level increased to toward the upper limit of normal physiologic activity (130%) at both 30 minutes post initiation of CPB (122%) and at the preprotamine time point (113%). In contrast, the AT III activity levels among the placebo patients requiring FFP were further reduced from baseline (74%) at both 30 minutes after initiation of CPB (52%) and at the preprotamine time point (51%).

The mean change from baseline to 30 minutes post initiation of CPB and the mean change from baseline to the preprotamine administration time point for AT III plasma activity level is provided in Table 12 for both treatment groups.

TABLE 12
AT III Plasma Activity Level - Mean Change from
Baseline (%) (Intent-To-Treat Population)

| | 30 Minutes Post CPB | | Preprotamine Administration | |
|---|---|---|---|---|
| Statistic | rhAT | Placebo | rhAT | Placebo |
| N | 25 | 26 | 25 | 26 |
| Mean | 43.1 | −22.2 | 34.3 | −23.1 |
| Std. Dev. | 27.5 | 15.3 | 29.7 | 17.7 |
| Minimum | −57 | −49 | −73 | −72 |
| Maximum | 85 | 19 | 84 | 6 |

| | | |
|---|---|---|
| P-Value | <0.001 | <0.001 |
| for Group | | |
| Differences | | |

Std. Dev. = Standard Deviation
Note:
Subject counts may vary due to missed sampling or samples that could not be assayed.
Note:
The normal range for plasma AT III activity is 70-130%.

A mean decrease from baseline in plasma AT III activity level was observed for the placebo plus FFP patients at both time points. In contrast, for patients who received rhAT without FFP, there was a mean increases in AT III activity level at both time points. The difference between the two treatment groups in the mean change from baseline was statistically significant at both time points (p<0.001). These observations and the large disparity in maximum AT III plasma activity levels between the two treatment groups as noted in Table 12 demonstrates that the administration of two units of FFP is not effective for increasing plasma AT III activity.

Cohort Analysis of Secondary Endpoints

There were four possible groupings of patients across the two study treatment groups. These included patients randomized to receive placebo that either required FFP or did not require FFP, and patients randomized to receive rhAT who either required or did not require FFP.

In terms of secondary efficacy endpoints, the study objective was to compare the effects of rhAT and FFP on AT III activity, thrombin activity, and fibrinolysis. Therefore, for each of these parameters, the relevant comparison is the group of placebo patients who required FFP administration to the group of rhAT patients who did not require FFP administration. In the Intent-To-Treat population, a total of 22 patients received placebo and subsequent FFP administration prior to the initiation of CPB, while 21 patients received rhAT without requiring FFP administration prior to initiation of CPB. The preprotamine time point for one placebo patient (Patient 04-023) has been excluded from the evaluation of secondary efficacy endpoints because the preprotamine blood samples for this patient were taken from pump blood after protamine administration. Tables 13 through 11-19 provide the descriptive statistics, the change from baseline by sampling time point for each of these two outcome cohorts, and the p-values for the between-group comparisons for D-Dimer, fibrin monomer, and AT III plasma activity levels. Intent-to-Treat actual value results are provided for each parameter, while change from baseline results are provided for both the Intent-To-Treat and the Per Protocol populations for each secondary efficacy endpoint.

D-Dimer Level

The effects of rhAT and FFP on thrombin activity were compared, as measured by the change in D-Dimer levels pre- and post-treatment. Table 13 provides the actual value descriptive statistics for D-Dimer levels at baseline, 30 minutes post initiation of CPB, and at the preprotamine time point for the placebo with FFP and the rhAT without FFP outcome cohorts.

TABLE 13
D-Dimer: Actual Value Descriptive Statistics
(ng/mL) (Intent-To-Treat Population)

| | | | 30 Minutes Post | | | |
|---|---|---|---|---|---|---|
| | Baseline | | Initiation of CPB | | Preprotamine | |
| | Placebo | rhAT | Placebo | rhAT | Placebo | rhAT |
| | with | without | with | without | with | without |
| Statistic | FFP | FFP | FFP | FFP | FFP | FFP |
| N | 21 | 21 | 21 | 20 | 21 | 20 |
| Mean | 143.9 | 117.8 | 636.0 | 687.9 | 1699.9 | 1353.0 |
| Std. Dev. | 151.7 | 177.5 | 1035.0 | 1094.2 | 1470.2 | 1769.7 |
| Minimum | 10 | 6 | 18 | 51 | 6 | 67 |
| Maximum | 436 | 782 | 4924 | 4356 | 6062 | 7097 |

| | | | |
|---|---|---|---|
| P-Value | 0.571 | 0.611 | 0.103 |
| for Group | | | |
| Differences | | | |

FFP = Fresh Frozen Plasma;
Std. Dev. = Standard Deviation;
P-values based on Rank Sum test
Note:
The normal range for D-Dimer is ≦100 ng/mL.
Note:
Patient counts may vary due to unobtainable assay results.

Using the Intent-To-Treat population, there were no statistically significant differences in mean D-Dimer levels between the two outcome cohorts at baseline, 30 minutes after initiation of CPB, or prior to protamine administration. Although not statistically significant (p=0.103), there was an apparent trend toward a smaller increase in D-Dimer levels from baseline at the preprotamine time point in the rhAT patients not requiring FFP compared to the placebo patients requiring FFP. The mean change in D-Dimer level from baseline to 30 minutes post initiation of CPB and to the preprotamine administration time point are summarized in Table 14.

TABLE 14
Mean Change from Baseline in D-Dimer Level
(ng/mL) (Intent-To-Treat Population)

| | 30 Minutes Post CPB | | Preprotamine Administration | |
|---|---|---|---|---|
| | Placebo | rhAT | Placebo | rhAT |
| | with | without | with | without |
| Statistic | FFP | FFP | FFP | FFP |
| n | 21 | 20 | 21 | 20 |
| Mean | 492.1 | 566.9 | 1556.0 | 1242.5 |
| Std. Dev. | 984.8 | 1029.7 | 1395.2 | 1711.9 |
| Minimum | −155 | −83 | −76 | 29 |
| Maximum | 4561 | 3994 | 5699 | 6735 |

| | | |
|---|---|---|
| P-Value | 0.725 | 0.083 |
| for Group | | |
| Differences | | |

FFP = Fresh Frozen Plasma;
Std. Dev. = Standard Deviation;
P-values based on Rank Sum test
Note:
The normal range for D-Dimer is ≦100 ng/mL.
Note:
Patient counts may vary due to unobtainable assay results.

Comparison of the mean change in D-Dimer level from baseline at 30 minutes post CPB and preprotamine administration also did not show any statistically significant differences between the placebo plus FFP and the rhAT without FFP cohorts using the Intent-To-Treat population. However, the mean D-Dimer level was lower in the rhAT without FFP cohort at the preprotamine time point, and the medians do demonstrate a consistent trend toward larger changes from baseline in D-Dimer levels at both time points for the placebo with FFP cohort [281 ng/ml versus 215 ng/ml (30 minutes post-CPB) and 1207 ng/ml versus 643 ng/ml (preprotamine)]. Graphic presentation of individual D-Dimer plasma level profiles for the two cohorts using the Intent-To-Treat Population is provided in FIG. 11-1.

Fibrin Monomer Level

The effects of rhAT and FFP on fibrinolysis were compared, as measured by the change in fibrin monomer levels pre- and post-treatment. Table 14 provides the actual value descriptive statistics for fibrin monomer levels at baseline, 30 minutes post initiation of CPB, and at the preprotamine time-point for the placebo with FFP and rhAT without FFP cohorts using the Intent-To-Treat population.

TABLE 14
Fibrin Monomer - Actual Value Descriptive
Statistics (nmol/L) (Intent-To-Treat Population)

| | | | 30 Minutes Post | | | |
|---|---|---|---|---|---|---|
| | Baseline | | Initiation of CPB | | Preprotamine | |
| | Placebo | rhAT | Placebo | rhAT | Placebo | rhAT |
| | with | without | with | without | with | without |
| Statistic | FFP | FFP | FFP | FFP | FFP | FFP |
| n | 20 | 18 | 18 | 18 | 20 | 16 |
| Mean | 32.0 | 30.3 | 27.6 | 27.0 | 31.9 | 27.6 |
| Std. Dev. | 12.7 | 13.1 | 10.2 | 12.2 | 11.2 | 11.0 |
| Minimum | 14 | 13 | 16 | 12 | 16 | 15 |
| Maximum | 56 | 53 | 57 | 49 | 55 | 53 |

| | | | |
|---|---|---|---|
| P-Value | 0.697 | 0.869 | 0.256 |
| for Group | | | |
| Differences | | | |

FFP = Fresh Frozen Plasma;
Std. Dev. = Standard Deviation;
P-values based on ANOVA
Note:
The normal range for fibrin monomer is ≦15 nmol/L.
Note:
Values reported as < or > have been excluded.
Note:
Patient counts may vary due to unobtainable assay results.

There was no statistically significant difference between the two outcome cohorts in mean fibrin monomer levels at baseline. Mean fibrin monomer levels were similar for each cohort at the 30 minutes after initiation of CPB and at the preprotamine time points. The mean changes in fibrin monomer levels from baseline for both cohorts at these two time points are summarized in Table 15.

TABLE 15
Mean Change from Baseline in Fibrin Monomer
Level (nmol/L) (Intent-To-Treat Population)

| | 30 Minutes Post CPB | | Preprotamine Administration | |
|---|---|---|---|---|
| | Placebo | rhAT | Placebo | rhAT |
| | with | without | with | without |
| Statistic | FFP | FFP | FFP | FFP |
| n | 17 | 16 | 19 | 14 |
| Mean | −3.2 | −1.2 | 1.2 | 2.4 |
| Std. Dev. | 9.0 | 9.7 | 10.1 | 6.5 |
| Minimum | −22 | −20 | −30 | −11 |
| Maximum | 15 | 14 | 22 | 17 |

| | | |
|---|---|---|
| P-Value | 0.555 | 0.692 |
| for Group | | |
| Differences | | |

FFP = Fresh Frozen Plasma;
Std. Dev. = Standard Deviation;
P-values based on ANOVA
Note:
The normal range for fibrin monomer is ≦15 nmol/L.
Note:
Values reported as < or > have been excluded.
Note:
Patient counts may vary due to unobtainable assay results.

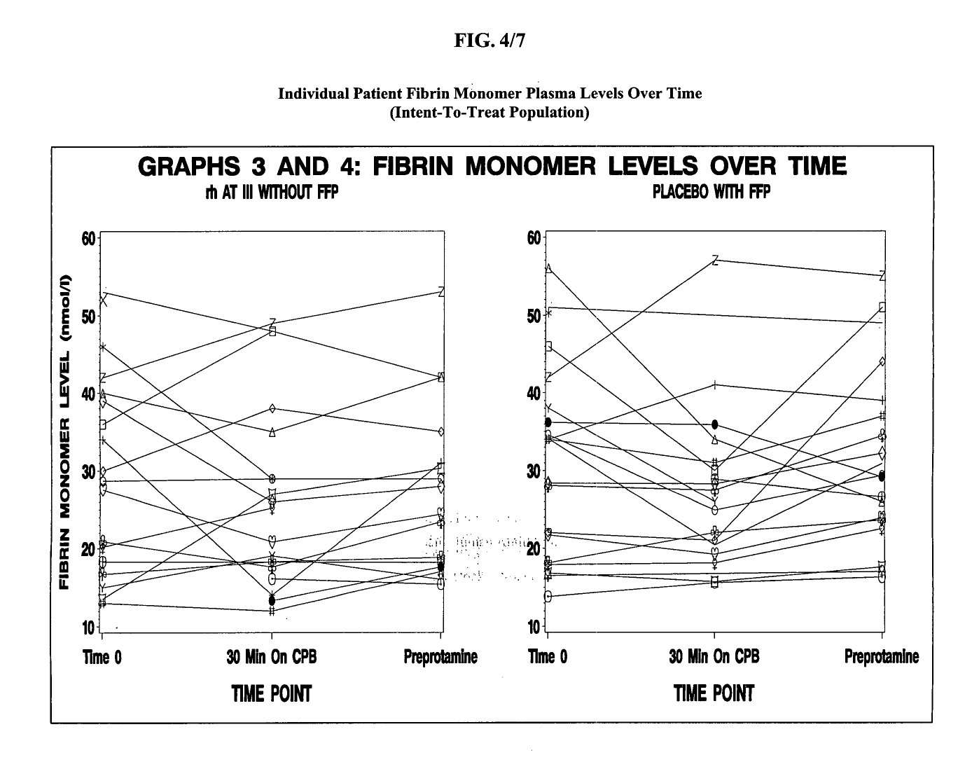

No statistically significant differences were noted between the two outcome cohorts in the mean change in fibrin monomer level from baseline at 30 minutes after initiation of CPB or at the preprotamine time point. Graphic presentation of individual fibrin monomer plasma level profiles for the two cohorts using the Intent-To-Treat population is provided in FIG. 4.

Mean change from baseline in fibrin monomer level at 30 minutes after initiation of CPB and at the preprotamine time point for the two outcome cohorts in the Per Protocol population are summarized in Table 16.

TABLE 16
Mean Change from Baseline in Fibrin Monomer
Level (nmol/L) (Per Protocol Population)

| | 30 Minutes Post CPB | | Preprotamine Administration | |
|---|---|---|---|---|
| | Placebo | rhAT | Placebo | rhAT |
| | with | without | with | without |
| Statistic | FFP | FFP | FFP | FFP |
| n | 14 | 13 | 16 | 12 |
| Mean | −1.9 | −2.0 | 3.2 | 1.8 |
| Std. Dev. | 7.6 | 10.4 | 7.0 | 6.6 |
| Minimum | −16 | −20 | −7 | −11 |
| Maximum | 15 | 14 | 22 | 17 |

| | | |
|---|---|---|
| P-Value | 0.991 | 0.579 |
| for Group | | |
| Differences | | |

FFP = Fresh Frozen Plasma;
Std. Dev. = Standard Deviation;
P-values based on ANOVA
Note:
The normal range for fibrin monomer is ≦15 nmol/L.
Note:
Values reported as < or > have been excluded.
Note:
Patient counts may vary due to unobtainable assay results.

In the Per Protocol population, there were no statistically significant differences between the two outcome cohorts in the mean change in fibrin monomer levels from baseline to 30 minutes after initiation of CPB or at the preprotamine time point.

Additionally, shifts (normal vs. abnormal) in fibrin monomer were also assessed so as to include trim values that were not incorporated into the analysis of actual values (Provided in Section 14.2). For both the Intent-To-Treat and Per Protocol populations, there were no significant differences between the two outcome cohorts with respect to shifts from baseline at either the 30 minutes post CPB initiation or preprotamine administration time points.

The meaning of these findings is not self-evident and must be considered with caution for the following reasons. When fibrin activates fibrinogen it forms a single fibrin molecule or fibrin monomer. During normal hemostasis, the fibrin monomer immediately polymerizes with other fibrin to form a clot and is not detectable in plasma. If fibrin is formed at a relatively low rate away from a polymerizing clot, fibrin can be released into the plasma where it forms a semi-stable complex with two fibrinogen molecules, resulting in plasma fibrin monomers or soluble fibrin. In addition, the level of fibrin measured in the plasma is dependent on the type of assay used to measure it.

The assay used in the current study was the Behring fibrin monomer assay. The principle of the assay is detection of fibrin-like fragments by measuring their ability to accelerate the activation of plasminogen by tissue plasminogen activator (TPA). Increased fibrin leads to increased plasmin, which produces increased yellow color in the reaction. Notably, the assay detects any fibrin that accelerates TPA. This would include true fibrin monomer as well as other materials such as fibrin oligomers (small polymers) and small fragments of degraded polymerized fibrin. As a result, the assay cannot be considered a true fibrin monomer assay.

Next, the effect of thrombin activity and fibrinolysis on the fibrin monomer assay must be considered. Increased thrombin activity can be the result of an increase in either hemostatic thrombin or non-specific thrombin. An increase in thrombin activity as the result of an increase in hemostatic thrombin yields little or no increase in fibrin monomer. An increase in thrombin activity due to an increase in non-specific thrombin increases fibrin monomer levels if the increase is low or moderate. If there is an increase in non-specific thrombin to high levels, fibrin monomer forms polymerized fibrin, which is unstable in plasma and not detectable.

When the fibrin level increases due to clot, increased fibrinolytic activity initially increases fibrin fragments, but later degrades fibrin, thus removing it from plasma. If the primary source of fibrin is soluble fibrin, increased fibrinolytic activity degrades fibrin and removes it from plasma.

In summary, fibrin monomer level in plasma is dependent on the level of hemostatic versus non-specific thrombin, the relative amount of fibrinolytic activity, the timing of sampling versus fibrinolytic activity, and methods used to stabilize the sample. Overall, because fibrin monomer is variably affected by thrombin and fibrinolytic activity and the assay procedure used was not specific for fibrin monomer, changes in the absolute amount of fibrin monomer over time may not have been accurately measured in either treatment group during the current study.

AT III Plasma Activity Level

The effect of rhAT and FFP on plasma AT III activity level were compared. Table 17 provides the actual value descriptive statistics for mean AT III plasma activity level at baseline, 30 minutes post initiation of CPB, and at the preprotamine time point for the placebo with FFP and the rhAT without FFP outcome cohorts using the Intent-to-Treat population.

TABLE 17
AT III Plasma Activity Level - Actual Value
Descriptive Statistics (%) (Intent-To-Treat Population)

| | | | 30 Minutes Post | | | |
|---|---|---|---|---|---|---|
| | Baseline | | Initiation of CPB | | Preprotamine | |
| | Placebo | rhAT | Placebo | rhAT | Placebo | rhAT |
| | with | without | with | without | with | without |
| Statistic | FFP | FFP | FFP | FFP | FFP | FFP |
| n | 21 | 21 | 21 | 20 | 21 | 20 |
| Mean | 73.7 | 76.3 | 51.3 | 122.7 | 51.5 | 113.6 |
| Std. Dev. | 15.2 | 25.4 | 12.3 | 19.5 | 13.6 | 32.4 |
| Minimum | 55 | 38 | 26 | 83 | 21 | 67 |
| Maximum | 116 | 140 | 83 | 159 | 76 | 204 |

| | | | |
|---|---|---|---|
| P-Value | 0.687* | <0.001* | <0.001** |
| for Group | | | |
| Differences | | | |

FFP = Fresh Frozen Plasma;
Std. Dev. = Standard Deviation
*P-value based on ANOVA
**P-value based on Rank Sum test
Note:
The normal range for plasma AT III activity is 70%-130% activity.
Note:
Patient counts may vary due to unobtainable assay results.

Using the Intent-To-Treat population, there was no statistically significant difference between the rhAT without FFP and the placebo with FFP cohorts in the mean plasma AT III activity level at baseline. The mean AT III activity level was above the lower limit of normal physiologic activity (70%) at baseline for patients in both cohorts (76% and 74%, respectively). However, there were statistically significant differences (p<0.001) between the two cohorts with respect to plasma AT III activity at 30 minutes post CPB initiation and preprotamine reversal. In the rhAT without FFP cohort, the mean plasma AT III activity level increased to toward the upper limit of normal physiologic activity (130%) at both 30 minutes post initiation of CPB (123%) and at the preprotamine time point (114%). In contrast, the AT III activity levels among the placebo patients requiring FFP were further reduced from baseline (74%) at both 30 minutes after initiation of CPB (51%) and at the preprotamine time point (52%).

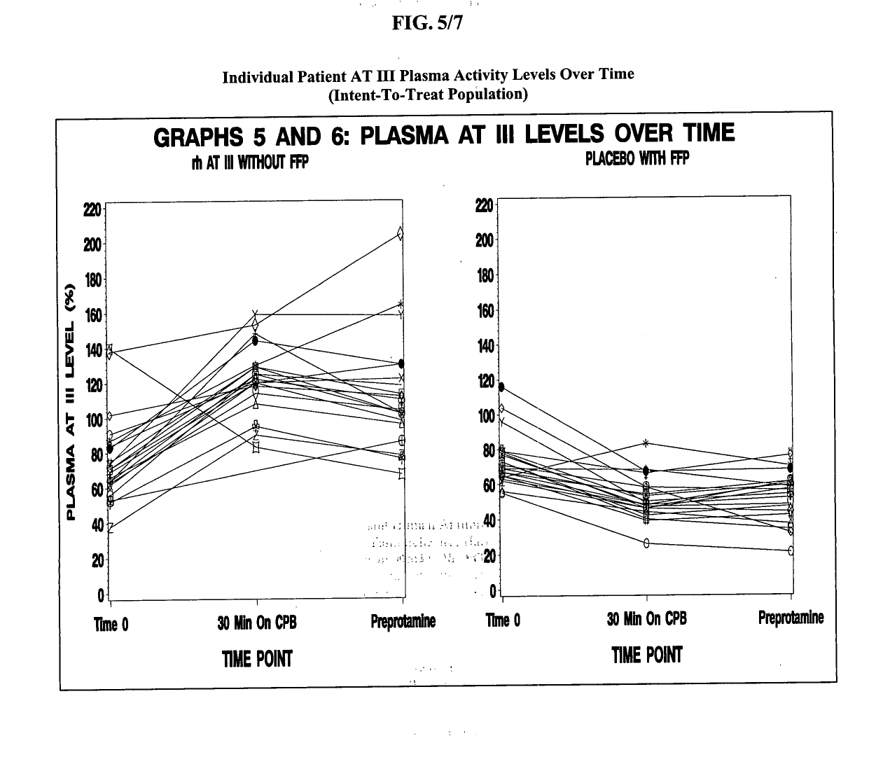

Graphic presentation of individual AT III plasma activity level profiles for the two cohorts using the Intent-To-Treat population is provided in FIG. 5.

Mean change from baseline in plasma AT III activity level at 30 minutes after initiation of CPB and at the preprotamine time point for the two outcome cohorts using the Intent-To-Treat population are summarized in Table 18.

TABLE 18
AT III Plasma Activity Level - Mean Change from
Baseline (%) (Intent-To-Treat Population)

| | 30 Minutes Post CPB | | Preprotamine Administration | |
|---|---|---|---|---|
| | Placebo | rhAT | Placebo | rhAT |
| | with | without | with | without |
| Statistic | FFP | FFP | FFP | FFP |
| n | 21 | 20 | 21 | 20 |
| Mean | −22.4 | 45.2 | −22.2 | 37.0 |
| Std. Dev. | 15.8 | 30.1 | 18.9 | 31.7 |
| Minimum | −49 | −57 | −72 | −73 |
| Maximum | 19 | 85 | 6 | 84 |

| | | |
|---|---|---|
| P-Value | <0.001** | <0.001* |
| for Group | | |
| Differences | | |

FFP = Fresh Frozen Plasma;
Std. Dev. = Standard Deviation
*P-value based on ANOVA
**P-value based on Rank Sum test
Note:
The normal range for plasma AT III activity is 70%-130%.
Note:
Patient counts may vary due to unobtainable assay results.

A mean decrease from baseline in plasma AT III activity level was observed for the placebo plus FFP patients at both time points. In contrast, for patients who received rhAT without FFP, there was a mean increases in AT III activity level at both time points. The difference between the two cohorts in the mean change from baseline was statistically significant at both time points (p<0.001). These observations and the large disparity in maximum AT III plasma activity levels between the two cohorts as noted in Table 18 demonstrate that the administration of two units of FFP is not effective for increasing plasma AT III activity.

Mean change from baseline in plasma AT III activity level at 30 minutes after initiation of CPB and at the preprotamine time point for the two outcome cohorts using the Per Protocol population is summarized in Table 19.

TABLE 19
AT III Plasma Activity Level - Mean Change from
Baseline (%) (Per Protocol Population)

| | 30 Minutes Post CPB | | Preprotamine Administration | |
|---|---|---|---|---|
| | Placebo | rhAT | Placebo | rhAT |
| | with | without | with | without |
| Statistic | FFP | FFP | FFP | FFP |
| n | 18 | 17 | 18 | 17 |
| Mean | −22.8 | 42.1 | −21.4 | 34.5 |
| Std. Dev. | 15.0 | 31.1 | 19.0 | 32.3 |
| Minimum | −49 | −57 | −72 | −73 |
| Maximum | 19 | 84 | 6 | 77 |

| | | |
|---|---|---|
| P-Value | <0.001* | <0.001** |
| for Group | | |
| Differences | | |

FFP = Fresh Frozen Plasma;
Std. Dev. = Standard Deviation
*P-value based on Rank Sum test
**P-value based on ANOVA
Note:
The normal range for plasma AT III activity is 70%-130%.
Note:
Patient counts may vary due to unobtainable assay results.

Results of the analysis of plasma AT III activity level using the Per Protocol population were similar to those using the Intent-To-Treat population.

Analyses: Other Measures of Thrombin Activation

Two additional indicators of thrombin activation-prothrombin fragment 1.2 and thrombin-antithrombin complex-were evaluated after completion of the study. These two parameters were measured at baseline (Time 0), at 30 minutes after initiation of CPB, and at the preprotamine time point in retained plasma samples. Measurement of prothrombin fragment 1.2 is an indicator of prothrombin consumption that occurs during its conversion to thrombin in the presence of factor Xa, factor Va, Ca⁺⁺, and a source of phospholipid (PF 3). A low level of prothrombin fragment 1.2 would correlate with reduced thrombin production.

The results of the measurement of prothrombin fragment 1.2 plasma levels for the rhAT and the placebo treatment groups using the Intent-To-Treat population are provided in Table 20.

TABLE 20
Mean Prothrombin Fragment 1.2 Plasma Levels
(nmol/L) (Intent-To-Treat Population)

| | | | | | P-Value |
|---|---|---|---|---|---|
| Sample | | | | | for Group |
| Time | Parameter | Statistic | Placebo | rhAT | Difference |
| Baseline | Actual | n | 26 | 26 | 0.304 |
| (Time 0) | Value | Mean | 1.1 | 1.1 | |
| | | Std. Dev. | 0.8 | 0.6 | |
| | | Min./Max. | 0.4/4.3 | 0.5/2.6 | |
| 30 Min. | Actual | n | 26 | 24 | ND |
| Post-CPB | Value | Mean | 2.2 | 3.2 | |
| | | Std. Dev. | 1.4 | 5.1 | |
| | | Min./Max. | 0.4/5.1 | 0.6/20.6 | |
| | Change | n | 26 | 26 | 0.377 |
| | from | Mean | 1.1 | 2.0 | |
| | Baseline | Std. Dev. | 1.6 | 5.0 | |
| | | Min./Max. | −2.2/4.2 | −0.8/19.3 | |
| Pre- | Actual | n | 25 | 24 | ND |
| protamine | Value | Mean | 7.4 | 5.7 | |
| | | Std. Dev. | 11.1 | 9.7 | |
| | | Min./Max. | 0.5/56.4 | 0.9/47.2 | |
| | Change | n | 25 | 24 | 0.134 |
| | from | Mean | 6.4 | 4.5 | |
| | Baseline | Std. Dev. | 11.3 | 9.6 | |
| | | Min./Max. | −0.8/55.9 | 0.2/45.9 | |

FFP = Fresh Frozen Plasma;
Std. Dev. = Standard Deviation;
Min./Max. = Minimum/Maximum;
ND = Not Done;
CPB = Cardiopulmonary Bypass;
P-values based on Rank Sum test
Note:
The normal range for prothrombin fragment 1.2 is 0.4-1.8 nmol/L.
Note:
Patient counts may vary due to unobtainable assay results.

Using the Intent-To-Treat population, there were no statistically significant differences between the two treatment groups for mean prothrombin fragment 1.2 levels at baseline or at 30 minutes post CPB initiation. However, the mean change from baseline at the preprotamine time point, although not a statistically significant difference (p=0.134), showed less of an increase in the rhAT treatment group compared to the group that received placebo (4.5 nmoles/L versus 6.4 nmoles/L, respectively).

The results of the measurement of prothrombin fragment 1.2 plasma levels for the rhAT without FFP and the placebo with FFP cohorts using the Intent-To-Treat population are provided in Table 21.

TABLE 21
Mean Prothrombin Fragment 1.2 Plasma Levels
(nmol/L) (Intent-To-Treat Population)

| | | | Placebo | rhAT | P-Value |
|---|---|---|---|---|---|
| Sample | | | with | without | for Group |
| Time | Parameter | Statistic | FFP | FFP | Difference |
| Baseline | Actual | n | 21 | 21 | 0.658 |
| (Time 0) | Value | Mean | 0.9 | 1.0 | |
| | | Std. Dev. | 0.4 | 0.6 | |
| | | Min./Max. | 0.4/2.2 | 0.5/2.5 | |
| 30 Min. | Actual | n | 21 | 19 | ND |
| Post-CPB | Value | Mean | 2.3 | 2.6 | |
| | | Std. Dev. | 1.5 | 3.9 | |
| | | Min./Max. | 0.4/5.1 | 0.6/17.8 | |
| | Change | n | 21 | 19 | 0.272 |
| | from | Mean | 1.4 | 1.6 | |
| | Baseline | Std. Dev. | 1.5 | 3.7 | |
| | | Min./Max. | −0.9/4.2 | −0.6/16.2 | |
| Pre- | Actual | n | 20 | 19 | ND |
| protamine | Value | Mean | 8.5 | 3.5 | |
| | | Std. Dev. | 12.3 | 4.0 | |
| | | Min./Max. | 0.5/56.4 | 0.9/17.5 | |
| | Change | n | 20 | 19 | |
| | from | Mean | 7.6 | 2.5 | 0.015 |
| | Baseline | Std. Dev. | 12.3 | 3.7 | |
| | | Min./Max. | −0.8/55.9 | 0.2/15.9 | |

FFP = Fresh Frozen Plasma;
Std. Dev. = Standard Deviation;
Min./Max. = Minimum/Maximum;
ND = Not Done;
CPB = Cardiopulmonary Bypass;
P-values based on Rank Sum test
Note:
The normal range for prothrombin fragment 1.2 is 0.4-1.8 nmol/L.
Note:
Patient counts may vary due to unobtainable assay results.

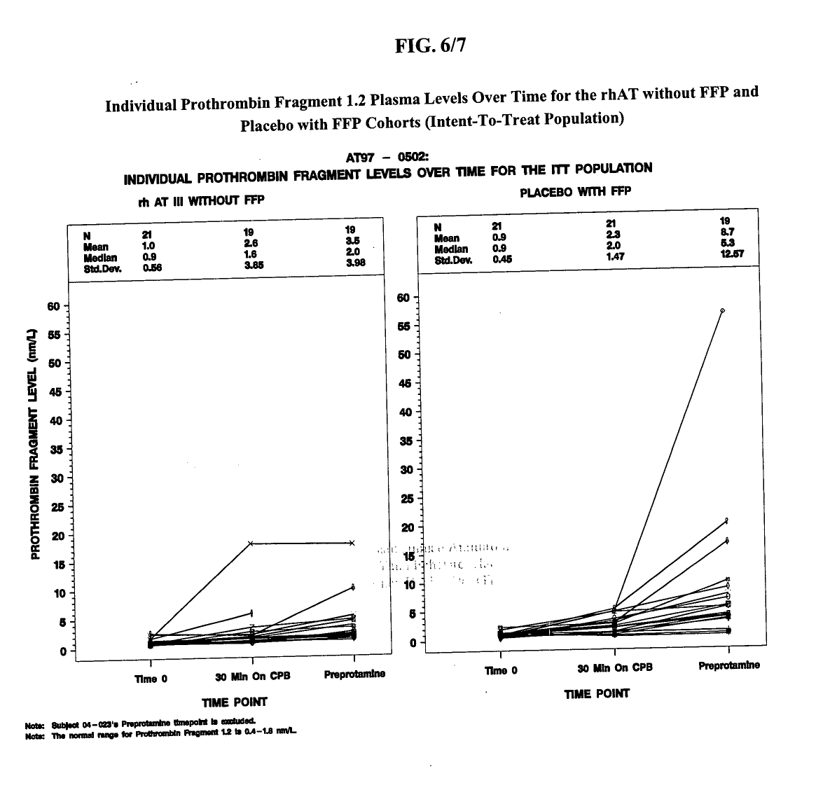

Using the Intent-To-Treat population, there were no statistically significant differences between the two outcome cohorts for mean prothrombin fragment 1.2 levels at baseline or at 30 minutes post CPB initiation. However, there was a statistically significant difference (p=0.015) between the outcome cohorts in the mean change in prothrombin fragment 1.2 levels from baseline to the preprotamine time point. The rhAT without FFP cohort demonstrated a significantly smaller increase in prothrombin fragment 1.2 levels when compared to the placebo with FFP cohort. The data also suggest that the effect observed in the rhAT without FFP group increased with time over the course of the surgery. FIG. 6 displays the individual prothrombin fragment 1.2 plasma levels over time for the rhAT without FFP cohort and the placebo with FFP cohort using the Intent-To-Treat population.

The level of thrombin-antithrombin complex will increase as thrombin is produced in the presence of antithrombin. The level of thrombin-antithrombin complex should be lower in the rhAT treatment group compared to the placebo treatment group if thrombin production was reduced. The results of the measurement of thrombin-antithrombin complex plasma levels for the placebo and the rhAT treatment groups using the Intent-To-Treat population are provided in Table 22.

TABLE 22
Mean Thrombin-Antithrombin Complex Plasma
Levels (ng/mL) (Intent-To-Treat Population)

| | | | | | P-Value |
|---|---|---|---|---|---|
| Sample | | | | | for Group |
| Time | Parameter | Statistic | Placebo | rhAT | Difference |
| Baseline | Actual | n | 26 | 26 | 0.487 |
| (Time 0) | Value | Mean | 12.3 | 11.0 | |
| | | Std. Dev. | 27.0 | 12.0 | |
| | | Min./Max. | 2.3/143.0 | 1.7/53.2 | |
| 30 Min. | Actual | n | 26 | 24 | ND |
| Post-CPB | Value | Mean | 53.9 | 61.4 | |
| | | Std. Dev. | 59.3 | 100.4 | |
| | | Min./Max. | 3.6/210.0 | 4.7/378.0 | |
| | Change | n | 26 | 24 | 0.593 |
| | from | Mean | 41.5 | 50.2 | |
| | Baseline | Std. Dev. | 69.1 | 102.4 | |
| | | Min./Max. | −125.9/206.5 | −28.2/374.4 | |
| Pre- | Actual | n | 25 | 24 | ND |
| protamine | Value | Mean | 109.1 | 86.0 | |
| | | Std. Dev. | 90.1 | 98.9 | |
| | | Min./Max. | 3.9/300.0 | 4.6/447.0 | |
| | Change | n | 25 | 24 | 0.211 |
| | from | Mean | 96.6 | 74.8 | |
| | Baseline | Std. Dev. | 98.1 | 99.4 | |
| | | Min./Max. | −86.0/293.3 | 1.7/443.4 | |

FFP = Fresh Frozen Plasma;
Std. Dev. = Standard Deviation;
Min./Max. = Minimum/Maximum;
CPB = Cardiopulmonary Bypass;
P-value based on Rank Sum test
Note:
The normal range for thrombin-antithrombin complex is 0.0-5.0 ng/mL.
Note:
Patient counts may vary due to unobtainable assay results.

Using the Intent-To-Treat population, there was no statistically significant difference between the two treatment groups in mean thrombin-antithrombin complex plasma levels at baseline, 30 minutes after initiation of CPB, or at the pre-protamine time point. However, at the preprotamine time point, the mean value and the mean increase in thrombin-antithrombin complex were less in the rhAT treatment group compared to the placebo group.

The results of the measurement of thrombin-antithrombin complex plasma levels for the placebo with FFP and the rhAT without FFP outcome cohorts using the Intent-To-Treat population are provided in Table 23.

TABLE 23
Mean Thrombin-Antithrombin Complex Plasma Levels (ng/mL)

| | | | Placebo | rhAT | P-Value |
|---|---|---|---|---|---|
| Sample | | | with | without | for Group |
| Time | Parameter | Statistic | FFP | FFP | Difference |
| Baseline | Actual | n | 21 | 21 | 0.642 |
| (Time 0) | Value | Mean | 7.1 | 9.7 | |
| | | Std. Dev. | 4.6 | 11.2 | |
| | | Min./Max. | 2.3/18.8 | 1.7/53.2 | |
| 30 Min. | Actual | n | 21 | 19 | ND |
| Post-CPB | Value | Mean | 62.5 | 53.4 | |
| | | Std. Dev. | 63.0 | 82.8 | |
| | | Min./Max. | 3.6/210.0 | 4.7/350.0 | |
| | Change | n | 21 | 19 | 0.417 |
| | from | Mean | 55.4 | 43.6 | |
| | Baseline | Std. Dev. | 64.6 | 83.3 | |
| | | Min./Max. | −13.3/206.5 | −28.2/340.8 | |
| Pre- | Actual | n | 20 | 19 | ND |
| protamine | Value | Mean | 123.6 | 57.8 | |
| | | Std. Dev. | 94.6 | 54.5 | |
| | | Min./Max. | 3.9/300.0 | 4.6/203.0 | |
| | Change | n | 20 | 19 | 0.019 |
| | from | Mean | 116.6 | 48.1 | |
| | Baseline | Std. Dev. | 96.0 | 52.8 | |
| | | Min./Max. | −3.6/293.3 | 1.7/193.8 | |

(Intent-To-Treat Population)
FFP = Fresh Frozen Plasma;
Std. Dev. = Standard Deviation;
Min./Max. = Minimum/Maximum;
CPB = Cardiopulmonary Bypass;
P-value based on Rank Sum test
Note:
The normal range for thrombin-antithrombin complex is 0.0-5.0 ng/mL.
Note:
Patient counts may vary due to unobtainable assay results.

Using the Intent-To-Treat population, there was no statistically significant difference between the two outcome cohorts in mean thrombin-antithrombin complex plasma levels at baseline. Similar to the results for prothrombin fragment 1.2 outcome cohort levels, there was a statistically significant difference (p=0.019) between the outcome cohorts in the mean change from baseline to the preprotamine time point for the thrombin-antithrombin complex levels. The rhAT without FFP cohort demonstrated a significantly smaller increase in thrombin-antithrombin complex levels than the placebo with FFP cohort. The mean change at the 30 minutes post CPB initiation time point was less in the rhAT without FFP group, but the change was not statistically significant (p=0.417); this may be due to a lack of sufficient time from the administration of study medication to see an effect. The data also suggest that the effect observed in the rhAT without FFP cohort increased with time over the course of the surgery.

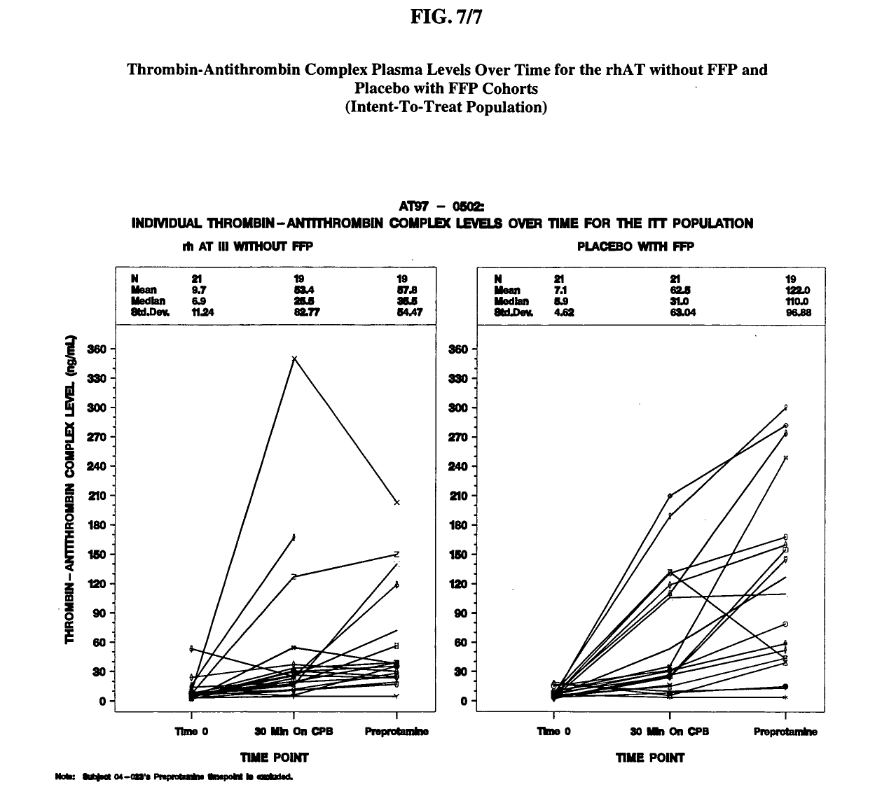

FIG. 7 displays the individual thrombin-antithrombin complex plasma levels over time for the rhAT without FFP cohort and the placebo with FFP cohort using the Intent-To-Treat population.

Effect of Study Medication on Subsequent Heparin Dosing

Although not a prospectively defined study endpoint, some interesting observations can be made regarding the administration of additional heparin during the study among the placebo with FFP patients and the rhAT without FFP patients (Section 14.2). In the Intent-To-Treat population, the mean total amount of heparin given while patients were on CPB was significantly greater (p=0.005; Rank Sum test) among placebo patients (n=22) who received FFP (6890 IU per hour) than among rhAT patients (n=21) who did not require FFP (3884 IU per hour).

However, it is arguably more relevant to look at the number of additional doses of heparin that were required in each of the two treatment groups (rhAT and placebo). Time frames of particular interest include 1) after administration of study medication but before CPB initiation, and 2) after administration of study medication up to the time of heparin reversal with protamine. These data are summarized in Table 24.

TABLE 24
Number of Patients Requiring Heparin Dosing After Administration
of Study Medication Prior to Initiation of CPB and Prior to Heparin
Reversal with Protamine (Intent-To-Treat Population)

| Treatment | | | | | | |
|---|---|---|---|---|---|---|
| Group | 0 | 1 | 2 | 3 | 4 | Total |
| | Number of Additional Heparin Doses After Administration | | | | | |
| | of Study Medication but Prior to Heparin Reversal with Protamine* | | | | | |
| Placebo | 6 | 12 | 3 | 5 | 1 | 21/27 |
| n | | | | | | (78%) |
| rhAT | 14 | 8 | 2 | 2 | 0 | 12/26 |
| n | | | | | | (46%) |
| | Number of Additional Heparin Doses After | | | | | |
| | Administration of Study Medication but Prior to CPB | | | | | |
| Placebo | 11 | 9 | 1 | 0 | 0 | 10/21** |
| n | | | | | | (48%) |
| rhAT | 9 | 3 | 0 | 0 | 0 | 3/12** |
| n | | | | | | (25%) |

*Does not include routine heparin priming of the CPB circuit
**The denominator is derived from the number of patients in each treatment group that required an additional dose of heparin after administration of study medication up to the time of heparin reversal with protamine.

A total of 21 out of 27 placebo patients (78%) required at least one additional dose of heparin some time between administration of study medication and heparin reversal with protamine. However, only 12 out of 26 patients (46%) treated with rhAT required at least one additional heparin dose during the same period. The most important observation among these patients is the distribution of the number of doses that were required across each treatment group. Twelve placebo patients and eight rhAT patients required only one additional dose of heparin. However, 9 placebo patients and only 4 rhAT patients required between 2 and 4 additional doses of heparin.

Among the 12 rhAT patients that required an additional heparin dose prior to heparin reversal with protamine only 3 (25%) of these patients required an additional dose of heparin before CPB, while 10 (48%) of the 21 placebo patients required additional heparin dosing prior to initiation of CPB. Additionally, the need for administration of more heparin doses among placebo patients may explain the cohort differences in mean time from the first dose of heparin to initiation of CPB. The mean time was 60 minutes for the rhAT patients not receiving FFP, while the mean time for placebo patients requiring FFP was 69 minutes (p=0.331).

All of the heparin dosing discussed above was administered by the attending physician in conjunction with the effort to maintain adequate anticoagulation and, therefore, qualify as an indicator of the impact of each of the study treatments. The clinical relevance of these findings is discussed further in Section 13.0 of this report.

Patient Disposition and Characteristics

Disposition and Accountability

All patients randomized into the study are included in the summary of patient disposition and accountability. No inferential statistical tests were performed. Frequencies and percentages of patients enrolled into the study, discontinued from the study, and completing the study by treatment group are summarized. For accountability, frequencies of patients (total and by study center) are provided at baseline, intra-operatively, and at the 4-week follow-up.

Demographics

Summary statistics by treatment group are provided for the following:
Age
Gender
Weight (in kg)
Ethnicity

Frequencies and percents are provided for gender and ethnicity categories, while descriptive statistics including N, mean, median, standard deviation, minimum, and maximum values are provided for age and weight.

Physical Exam and Medical History

Frequencies and percents by treatment group are provided for all 12 categories of the physical exam and all 16 categories of the medical history taken pre-operatively. Items are classified as ‘normal,’ ‘abnormal,2 or ‘not examined.’ In addition, pre-operative vital signs including blood pressure, heart rate, and weight are summarized with descriptive statistics including N, mean, median, standard deviation, minimum, and maximum values. No inferential tests were performed.

Efficacy Endpoints

The primary efficacy analysis was based on the Intent-To-Treat population and included all randomized patients who received the blinded study drug (rhAT Patient 02-035 was excluded from the ITT population because study drug administration was stopped after only 1 mL because the surgeon decided to perform an “off pump” procedure).

Primary Efficacy Endpoint

Treatment group differences in the proportion of patients requiring the administration of FFP were to be assessed using the Pearson Chi-square test with treatment group and FFP status as factors. However, the assumptions of this test were not met; therefore, Barnard's Unconditional Exact Hypothesis Test for the null hypothesis, H₀:pₜᵣₜ=P_{cont}, was employed, as allowed for in the Statistical Analysis Plan. Due to the relatively large number of centers involved in this protocol, the small number of patients recruited at each center, and the standardized measuring of ACTs among centers, center-to-center variation was not assessed.

Secondary Efficacy Endpoints

Treatment group and cohort differences in changes from baseline (time 0) in plasma levels of Fibrin Monomer, D-Dimer, and plasma AT III activity level at 30 minutes post-CPB initiation and preprotamine were assessed using an Analysis of Variance (ANOVA) model when assumptions were met. When assumptions were not met, a Rank Sum test was used. Since secondary endpoints were determined from assays performed by a single central lab, study center variation and center/treatment interaction were not assessed.

In addition to the exact value summaries for the fibrin monomer data, shift tables were also provided, due to the large number of trim values reported as > or < some value for the fibrin monomer levels. The percentage of patients in each treatment group shifting from normal to abnormal at both the 30 minutes on CPB and just prior to protamine reversal time points were compared. Results were commensurate with the exact value analyses (Section 14.2).

Safety Endpoints

All analyses of safety parameters were conducted on the safety population. The safety population was comprised of all patients who met the inclusion/exclusion criteria, signed an informed consent, and were randomized to a treatment arm.

Adverse Events

All adverse experiences are summarized by body system and by preferred term based on the WHOART coding dictionary. Frequencies and percents of treatment-emergent adverse events by treatment group are tabulated.

Tables are presented for treatment-emergent adverse events by intensity or severity and by relationship to the treatment and/or concomitant medications. The intensity has three categories: mild, moderate, or severe. The most severe intensity was assigned to a patient when more than one occurrence of the same AE was reported. Relationships of the AE to treatment and concomitant medications are categorized as ‘not related,’ ‘possible,’ ‘probable,’ or ‘definite.’ The highest level of association was reported in patients with differing relationships for the same AE. Listings of the above, as well as actions taken regarding treatment, episode type, and patient outcome, are provided for all patients. Separate listings for patients who withdrew from the study due to AEs, as well as all patients reporting serious AEs, are also provided.

In addition to the adverse event tables and listings, it was decided, in consultation with Clinical and Medical Affairs and Pharmacovigilance, to compare the proportion of patients reporting specific events in each treatment group for the most frequently reported adverse events. The incidences of the adverse events that occurred in at least 10% of either or both treatment groups were compared.

Tabulation of Individual Response Data

Individual patient response data for the single primary efficacy endpoint, and the three secondary efficacy endpoints, are provided in Appendix 16.2.6. These tabulations are for the Intent-To-Treat population and are displayed by treatment group.

Drug Dose, Drug Concentration, and Relationships to Response

Direct measurement of rhAT was not performed because of the unavailability of an assay procedure that could distinguish between endogenous AT III and rhAT in blood plasma. Further, this study was not designed to evaluate dose response or to determine the pharmacokinetics of rhAT. However, the change in blood plasma AT III levels after the administration of the single dose of study medication was a secondary efficacy endpoint of the study.

Drug-Drug and Drug-Disease Interactions

This study was not intended to evaluate drug-drug or drug-disease interactions. However, no apparent relationships between response and concomitant therapy, and between response and past and/or concurrent illness were observed.

Efficacy Conclusions

This study established that treatment with rhAT significantly reduced the requirement for administration of fresh frozen plasma per protocol to heparin resistant CPB patients in order to achieve an ACT≧480 seconds. Heparin resistance in this study was defined as having failed to achieve an ACT≧480 seconds within 5 minutes after intravenous administration of a total of 400 U/kg of heparin. Specifically, the proportion of rhAT treated patients in the Intent-To-Treat population who required administration of two units of FFP to achieve an activated clotting time ≧480 seconds was significantly less than the proportion of placebo treated patients who required two units of FFP (p<0.001). Similar results for this primary efficacy endpoint were seen in the Per Protocol population (p<0.001).

Further support of the efficacy of rhAT was provided by one of the three secondary study endpoints. Treatment with rhAT produced a significant increase in plasma AT III activity levels, while AT III activity levels among placebo treated patients continued to decline. The differences in AT III activity levels between the two treatment groups were statistically significant (p<0.001) at both time points (30 minutes on CPB and preprotamine administration) and for both the Intent-To-Treat and the Per Protocol populations. The same result was observed when the two treatment cohorts (rhAT without FFP and placebo with FFP) were compared at each time point and for each study population.

For the Intent-To-Treat population, there were no statistically significant differences in mean change in D-Dimer levels or in fibrin monomer levels between the two treatment groups at 30 minutes after initiation of CPB or just prior to protamine administration. The same result was obtained when the Per Protocol population was analyzed. In addition, no statistically significant differences were observed for either parameter using either patient population when the rhAT without FFP and the placebo with FFP cohorts were compared. However, for D-Dimer, a trend toward smaller increases in levels was observed for the cohort analysis at the preprotamine time point for both the Intent-To-Treat (p=0.083) and the Per Protocol (p=0.067) populations.

Several exploratory efficacy evaluations were performed. Among these evaluations was a comparison of the effect of placebo versus rhAT on activated clotting time (ACT). The mean change in ACT value from baseline (post heparinization) to 5 minutes after study drug administration was significantly greater (p<0.001) for the rhAT patients compared to the placebo patients. This result demonstrates that rhAT administration at 75 U/kg has a more rapid and clinically relevant effect on the ACT in heparin resistant patients than administration of two units of FFP. A significant difference in activated clotting time between treatment groups was also observed at the 30 minutes after initiation of CPB time point (p<0.001) and the preprotamine time point (p=0.003).

Another exploratory efficacy evaluation retrospectively examined two additional markers of thrombin activation: prothrombin fragment 1.2 and antithrombin-thrombin complex. Compared to placebo patients, patients who received rhAT showed significant inhibition of the generation of prothrombin fragment 1.2 (p=0.020) and thrombin-antithrombin complex (p=0.029) from baseline levels at the time just prior to reversal with protamine.

The effect of study medication on subsequent heparin dosing was also assessed. Seventy-eight percent (78%) of placebo patients required at least one additional dose of heparin some time between administration of study medication and heparin reversal with protamine, while only 46% of patients treated with rhAT required at least one additional heparin dose during the same period.

Based on having achieved the primary efficacy endpoint, rhAT at a dose of 75 U/kg effectively restores the anticoagulant activity of heparin in heparin resistant patients, thereby avoiding the use of FFP to achieve an ACT>480 seconds. This finding was further supported by the normalization of serum AT III levels among patients administered rhAT for the duration of CPB. In addition, although not an efficacy endpoint that could be prospectively defined, the statistically significant difference in activated clotting time (ACT) between rhAT patients and placebo patients is consistent with the primary endpoint result and circulating AT III levels. Finally, retrospective evaluations of two additional biochemical markers of thrombin activation, prothrombin fragment 1.2 and thrombin-antithrombin complex, support the prospectively-defined efficacy evaluations and provide a more reliable measure of thrombin production and activity than fibrin monomer levels, where no drug effect was observed.

Clinical Laboratory Evaluation

Listing of Individual Laboratory Measurements by Patient and Each Abnormal Laboratory Value

Individual patient laboratory values by study center, including blood chemistry, hematology, urinalysis, and the normal ranges are provided herein. Individual patient AT III antibody test results, measured as either positive (antibody present) or negative (antibody not present), are also provided herein. Because of the small amount of study drug (1 mL) administered to rhAT patient 02-035, laboratory tests were not performed for this patient. Post-operative laboratory specimens were not obtained from rhAT patient 13-197 prior to his death.

Evaluation of Each Laboratory Parameter

Laboratory Values Over Time

Blood chemistry, hematology, and urinalysis parameters were monitored at baseline and at 24 to 48 hours post-operatively or prior to discharge from the hospital, whichever occurred first. The mean changes in the individual laboratory parameters were not reviewed; however, shifts from normal at baseline to abnormal post-operatively were reviewed below. In addition, selected post-operative laboratory values that met the criteria for the Eastern Cooperative Oncology Group (ECOG) grade 3+ toxicity were identified retrospectively and evaluated.

### Individual Patient Changes

Patient Shifts from Normal to Abnormal

Examination of the shift from normal values at baseline to post-operative abnormal values (either above the upper limit of normal or below the lower limit of normal) by treatment group for each laboratory parameter provides a reasonable estimation of any dramatic effects between the treatment groups. Tables Table 25, Table 26, and Table 27 below provide the percent of patients whose laboratory value shifted from normal at baseline to abnormal post-operatively for blood chemistry, hematology, and urinalysis parameters, respectively.

Blood chemistry, hematology, and urinalysis results did not appear to reveal any safety concerns associated with rhAT treatment. The single-dose study design, the size of the study population in both treatment groups, the underlying patient disease status, and the impact of the surgical procedures that were performed make it difficult to differentiate treatment-related effects on these laboratory measurements.

Because of the study conditions described above, statistical comparisons between the rhAT and placebo groups were not performed for any of the blood chemistry, hematology, or urinalysis parameters measured during the study. Treatment group shifts for blood chemistry parameters are displayed in Table 25.

TABLE 25
Blood Chemistry - Shifts from Normal at Baseline
to Abnormal Post-operatively (Safety Population)

| | rhAT (n = 27) | | | Placebo (n = 27) | | |
|---|---|---|---|---|---|---|
| | Baseline | Post-Op | % Shift | Baseline | Post-Op | % Shift |
| | Normal | Abnormal | Toward | Normal | Abnormal | Toward |
| Parameter | n | n | Abnormal | n | n | Abnormal |
| ALAT (SGPT) | 21 | 1 | 4 | 25 | 2 | 7* |
| Albumin | 14 | 6 | 22* | 14 | 4 | 15 |
| Alkaline Phosphatase | 23 | 0 | 0 | 27 | 1 | 4* |
| ASAT (SGOT) | 22 | 6 | 22* | 27 | 5 | 18 |
| Bilirubin, Total | 24 | 2 | 7 | 26 | 3 | 11* |
| Calcium | 8 | 3 | 11 | 10 | 6 | 22* |
| Carbon Dioxide (CO2) | 8 | 0 | 0 | 7 | 1 | 4* |
| Chloride | 22 | 5 | 18 | 19 | 6 | 22* |
| CO₂ Content | 12 | 1 | 4* | 15 | 0 | 0 |
| CPK, Total | 24 | 23 | 85 | 27 | 24 | 89* |
| Creatinine | 22 | 5 | 18* | 24 | 2 | 7 |
| Glucose | 11 | 9 | 33 | 19 | 15 | 56* |
| LDH | 24 | 14 | 52* | 26 | 13 | 48 |
| Phosphorous Inorganic | 21 | 8 | 30 | 26 | 8 | 30 |
| Potassium | 25 | 1 | 4 | 27 | 4 | 15* |
| Protein, Total Serum | 11 | 10 | 37* | 6 | 6 | 22 |
| Sodium | 25 | 4 | 15 | 25 | 5 | 18* |
| Urea Nitrogen | 25 | 4 | 15 | 27 | 8 | 30* |
| Uric Acid | 23 | 2 | 7 | 25 | 5 | 18* |

Percentages are based on the total number of patients in each treatment group.
ALAT = Alanine Aminotransferase;
ASAT = Aspartate Aminotransferase;
CPK = Creatinine Phosphokinase;
LDH = Lactate Dehydrogenase
*Designates the group in which the percentage of patients that shifted to abnormal was greater.

Of the 19 blood chemistry parameters that were measured, the percentage shift toward abnormal was greater among the placebo patients for 12 parameters, while the greater percentage shift occurred in rhAT patients for 6 parameters. There was no difference between the treatment groups for the remaining parameter (inorganic phosphorus). The most pronounced treatment differences observed in the blood chemistry profile were greater percentage shifts (from normal baseline values to abnormal post-operative values) in the placebo group for glucose (56% vs. 33% in rhAT group) and urea nitrogen (30% vs. 15%), and a greater shift in the rhAT group for total serum protein (37% vs. 22% in the placebo group). The shifts for glucose were to values above normal for all placebo and rhAT patients listed, while the shifts for total serum protein were to values below normal for all patients listed. The shifts for urea nitrogen were to values above normal for 7 placebo patients and all 4 rhAT patients, with one placebo patient having a shift to below normal. Treatment group shifts for hematology parameters are displayed in Table 26.

TABLE 26
Hematology - Shifts from Normal at Baseline to
Abnormal Post-operatively (Safety Population)

| | rhAT (n = 27) | | | Placebo (n = 27) | | |
|---|---|---|---|---|---|---|
| | Baseline | Post-Op | % Shift | Baseline | Post-Op | % Shift |
| | Normal | Abnormal | Toward | Normal | Abnormal | Toward |
| Parameter | n | n | Abnormal | n | n | Abnormal |
| White Cell Ct. | 21 | 8 | 30 | 23 | 13 | 48* |
| Platelet Count | 18 | 5 | 18 | 23 | 9 | 33* |
| Hemoglobin | 7 | 7 | 26* | 4 | 4 | 15 |
| Hematocrit | 4 | 4 | 15* | 3 | 3 | 11 |
| Eosinophils | 22 | 0 | 0 | 16 | 0 | 0 |
| Basophils | 23 | 0 | 0 | 21 | 0 | 0 |
| Monocytes | 23 | 0 | 0 | 21 | 0 | 0 |
| Lymphocytes | 20 | 17 | 63* | 17 | 16 | 59 |
| Neutroph. Seg. | 20 | 15 | 56* | 17 | 13 | 48 |
| MCV | 23 | 0 | 0 | 25 | 0 | 0 |
| MCH | 20 | 1 | 4* | 24 | 0 | 0 |
| MCHC | 23 | 0 | 0 | 25 | 0 | 0 |

Percentages are based on the total number of patients in each treatment group.
Ct. = Count;
Neutroph. = Neutroph. Seg. = neutrophils segmented;
MCV = Mean Corpuscular Volume;
MCH = Mean Corpuscular Hemoglobin;
MCHC = Mean Corpuscular Hemoglobin Concentration
*Designates the group in which the percentage of patients that shifted to abnormal was greater.

Among the 12 parameters that were measured as part of hematology, the percentage shift toward abnormal was greater among the rhAT patients for 5 parameters, while the greater percentage shift occurred in placebo patients for 2 parameters. There was no difference between the treatment groups for the remaining 5 parameters. The greater shift toward abnormal among rhAT patients for hemoglobin and hematocrit involved too few patients in each treatment group to be considered clinically relevant. The larger shifts (18-48%) for both white blood cell count (all to above normal) and platelet count (all to below normal) in both treatment groups are not unexpected physiologic responses to the patients' surgical procedures. Likewise, the significant shifts (≧48%) for both lymphocytes (all to below normal values) and segmented neutrophils (all to above normal values) in both treatment groups are not unexpected immunologic responses to the patients' surgeries. The hematology data are difficult to interpret because of the many confounding factors associated with each patient's underlying disease, the complexity of the surgical procedures, and the frequent administration of various blood products to the majority of patients in each treatment group. Treatment group shifts for urinalysis parameters are displayed in Table 27.

TABLE 27
Urinalysis - Shifts from Normal at Baseline to
Abnormal Post-operatively (Safety Population)

| | rhAT (n = 27) | | | Placebo (n = 27) | | |
|---|---|---|---|---|---|---|
| | Baseline | Post-Op | % Shift | Baseline | Post-Op | % Shift |
| | Normal | Abnormal | Toward | Normal | Abnormal | Toward |
| Parameter | n | n | Abnormal | n | n | Abnormal |
| Appearance | 18 | 1 | 4 | 22 | 9 | 33* |
| Color | 24 | 2 | 7 | 23 | 2 | 7 |
| Specific Gravity | 24 | 1 | 4 | 24 | 1 | 4 |
| Reaction pH | 25 | 0 | 0 | 24 | 0 | 0 |
| Glucose | 20 | 1 | 4 | 22 | 1 | 4 |
| Bilirubin | 25 | 0 | 0 | 24 | 0 | 0 |
| Ketone | 24 | 1 | 4 | 23 | 2 | 7* |
| Nitrite | 24 | 0 | 0 | 23 | 1 | 4* |
| Occult Blood | 18 | 8 | 30 | 20 | 8 | 30 |
| Protein | 13 | 4 | 15 | 17 | 6 | 22* |
| RBC/HPF | 19 | 6 | 22 | 22 | 7 | 26* |
| WBC/HPF | 22 | 2 | 7 | 23 | 4 | 15* |

RBC/HPF = Red Blood Cells/High Powered Field;
WBC/HPF = White Blood Cells/High Powered Field
Percentages are based on the total number of patients in each treatment group.
*Designates the group in which the percentage of patients that shifted to abnormal was greater.

Among the 12 parameters that were measured as part of urinalysis, the percentage shift toward abnormal was greater among the placebo group for 6 parameters. There was no difference between the treatment groups for the other 6 parameters. None of the differences between the two treatment groups appeared to be clinically relevant.

ECOG Grade 3+ Laboratory Values

In addition to the shifts in laboratory values discussed above, selected post-operative laboratory values that met the Eastern Cooperative Oncology Group (ECOG) grade 3+ toxicity criteria after administration of study drug were identified retrospectively. These criteria are displayed in Table 28.

TABLE 28
Eastern Cooperative Oncology Group (ECOG) Grade 3+
Toxicity Criteria for Selected Laboratory Parameters

| Parameter | ECOG Grade 3+ Toxicity Criterion |
|---|---|
| Alanine Aminotransferase (ALAT) | 5 times the upper limit of normal |
| Alkaline Phosphatase | 5 times the upper limit of normal |
| Aspartate Aminotransferase (ASAT) | 5 times the upper limit of normal |
| Urea Nitrogen | 5 times the upper limit of normal |
| Bilirubin | 1.5 times the upper limit of normal |
| Creatinine | 3 times the upper limit of normal |
| White Blood Cell Count | Values less than 2.0 × 10³/mm³ |
| Platelet Count | Values less than 50,000/μL |

Abnormalities meeting the ECOG grade 3+ criteria provided in Table 28 were observed for ALAT, creatinine, total bilirubin, and platelet count. These abnormalities involved 2 placebo patients and 4 rhAT patients. Table 29 lists each of the abnormal values by patient by treatment group and includes the baseline value for each patient.

TABLE 29
Patients Having Selected Post-operative Laboratory Values
Meeting ECOG Grade 3+ Toxicity Criteria (Safety Population)

| Treatment | Patient | Laboratory | Study | | Normal |
|---|---|---|---|---|---|
| Group | Identification | Parameter | Period | Value | Range |
| Placebo | 10-151 | Creatinine | Baseline | 1.8 | 0.5-1.4 |
| | | (mg/dL) | Postoperative | 4.6 | |
| | Table 269 | Bilirubin, | Baseline | 0.7 | ≦1.3 |
| | | Total (mg/dL) | Postoperative | 2.9 | |
| rhAT | 04-024 | ALAT | Baseline | 262.0 | ≦48 |
| | | (U/L) | Postoperative | 80.0 | |
| | 06-082 | Bilirubin, | Baseline | 0.4 | ≦1.3 |
| | | Total (mg/dL) | Postoperative | 3.2 | |
| | 10-148 | Bilirubin, | Baseline | 1.5 | ≦1.3 |
| | | Total (mg/dL) | Postoperative | 2.3 | |
| | Table 273 | Platelet Count | Baseline | 122000 | 130000-400000 |
| | | (/μL) | Postoperative | 44000 | |

ALAT = Alanine Aminotransferase

There were no statistically significant differences (p>0.05) between the two treatment groups in the percentages of patients who demonstrated any post-operative laboratory value at or above the ECOG Grade 3 toxicity level. Among these patients, one placebo patient and three rhAT patients each had a single elevated liver enzyme. Therefore, these results are not suggestive of hepatotoxicity. The remaining findings—elevated creatinine in one placebo patient and decreased platelet count in one rhAT patient—are not considered indicative of a clinical trend and are unlikely related to the study medication that was administered.

AT III Antibody Testing and Retrospective Viral Testing Sample Collection

Serum antibody testing (ELISA) and the collection of a serum sample for retrospective viral screening were performed at baseline (i.e., just prior to study drug administration) and again approximately 4 weeks post-operatively. ELISA testing was employed for initial screening for rhAT antibody formation (IgG seroconversion) for all patients in both the rhAT group and the placebo group. A positive ELISA result required confirmation using a radio-immunoprecipitation (RIP) assay.

ELISA results were negative (i.e., within normal range) for all patients in both treatment groups at both baseline and 4 weeks post-operatively. Seroconversion was not observed for any patient based on the ELISA results. It should be noted that antibody testing at 4 weeks post-operatively was not done for 5 placebo patients and 4 rhAT patients, so seroconversion status for these 9 patients is unknown.

Serum samples for retrospective viral screening were collected in view of rhAT being derived from the milk of transgenic goats and the unknown potential for transmission of virus of animal (goat) origin. No safety issue has prompted analysis of the samples. The samples are being stored at the The Inventors Corporation facility in Framingham, Mass., where they will be kept for a period of two years (until at least July 2001).

Individual Clinically Significant Changes

One patient in each treatment group had abnormal laboratory values that were reported as serious adverse events.

Patient 10-145, who received placebo, had pre-existing anemia at the time of surgery (hemoglobin value of 8.5 g/dL), which was reported in association with bleeding and was considered not related to study medication.

Patient 04-024, who received rhAT, experienced coagulopathy post-operatively, as well as acute renal failure demonstrated by worsening BUN and creatinine levels. These serious adverse events, which were considered not related to study drug, occurred concurrently with worsening cardiac failure, from which the patient ultimately died.

Vital Signs, Physical Findings, and Other Observations Related to Safety

Vital Signs

Systolic and diastolic blood pressure and heart rate were measured at baseline (just prior to administration of study medication) and again at 1, 5, and 10 minutes after study medication administration. No other vital sign measures were recorded during the study. The collection of all vital sign values occurred after anesthesia induction, surgical incision, and heparinization had been performed but prior to the surgeon's decision regarding whether or not to administer FFP. The single-dose study design, the size of the study population in both treatment groups, the underlying patient disease status, and the fact that the surgical procedures were in progress at the time of data collection make it difficult to differentiate treatment-related effects on vital sign measurements. The mean changes from baseline for vital signs at each time point are provided in Table 30.

TABLE 30
Mean Changes from Baseline for Vital Signs Post-Dosing (Safety Population)

| | | Systolic Blood Pressure | | | | Diastolic Blood Pressure | | | | | | | |
|---|---|---|---|---|---|---|---|---|---|---|---|---|---|
| Time | | (mmHg) | | | | (mmHg) | | | | Heart Rate (bpm) | | | |
| Post | | | rh AT | | | | rh AT | | | | rh AT | | |
| Dose | | n | III | n | Placebo | n | III | n | Placebo | n | III | n | Placebo |
| 1 | min. | 24 | −0.2 | 27 | 0.1 | 24 | −0.7 | 27 | −0.7 | 24 | 0.2 | 27 | 0.6 |
| 5 | min. | 26 | −0.5 | 26 | −1.2 | 26 | −0.2 | 26 | 0.6 | 26 | 1.6 | 26 | 3.4 |
| 10 | min. | 24 | −3.2 | 22 | −3.0 | 24 | −1.1 | 23 | 1.9 | 24 | 1.1 | 23 | 2.1 |

All mean changes from baseline in vital signs post-dosing in both treatment groups at every time point were small in magnitude. No acute change in blood pressure or heart rate was observed in conjunction with administration of rhAT (measurements taken within 10 minutes of dosing). None of these mean changes was considered to be clinically relevant, and none of the differences between treatment groups was statistically significant (ANOVA, p>0.05).

Physical Findings

A physical examination was performed at the baseline visit only. Baseline physical examinations were similar for the two treatment groups, and were consistent with and reflective of a patient population with pre-existing cardiovascular disease. Three patients randomized to receive rhAT and five patients randomized to receive placebo had normal baseline physical exams. All remaining patients in both treatment groups had one or more abnormalities. None of these abnormalities was considered by the respective investigators to be exclusionary for study participation.

Electrocardiograms (ECG) were performed pre- and post-operatively. Only 2 of the 27 placebo patients and 6 of the 27 rhAT patients had a normal ECG pre-operatively. Post-operatively, only 2 of the 27 placebo patients and 6 of the 27 rhAT patients had normal ECG results. Only one patient, in the placebo group, had a normal ECG pre-operatively and an abnormal ECG post-operatively. Patient 04-023 developed a lateral injury but had normal sinus rhythm. There were no statistically significant shifts in ECG status within either treatment group. The general ECG findings are not unexpected based on the patient population, patient age, and medical history.

Other Observations Related to Safety

Several prospectively defined parameters relating to safety were recorded. These included the length of stay in the SICU, the length of hospitalization, and post-operative chest tube drainage volume (a potential measure of increased postoperative bleeding). These findings are displayed in Table 31.

TABLE 31
Patient Outcome Measures Related
to Safety - Quantitative Measures

Treatment Group

| Parameter | Statistic | rhAT* | Placebo |
|---|---|---|---|
| Length of | n | 25 | 27 |
| Stay in the | Mean | 3.8 | 2.5 |
| SICU | Standard Deviation | 8.8 | 2.7 |
| (Days) | Minimum/Maximum | 0/45 | 1/11 |
| Length of | n | 25 | 27 |
| Hospitalization | Mean | 11.8 | 10.6 |
| (Days) | Standard Deviation | 17.0 | 7.5 |
| | Minimum/Maximum | 3/91 | 4/34 |
| Chest | n | 27 | 26 |
| Tube Drainage - | Mean | 124 | 71 |
| Period 12 Hours | Standard Deviation | 97 | 42 |
| Post-op. (mL/hr) | Minimum/Maximum | 8/429 | 14/217 |
| Chest | n | 27 | 26 |
| Tube Drainage - | Mean | 86 | 52 |
| Period 24 Hours | Standard Deviation | 59 | 30 |
| Post-op. (mL/hr) | Minimum/Maximum | 11/263 | 13/142 |

*Patient 04-024 died in SICU, and Patient 13-197 died postoperatively.
Note:
The completion time of placebo patient 10-145's surgery is unknown.

Small differences between treatment groups were observed for the mean SICU stay and the total length of hospitalization. In both cases, rhAT patients stayed longer than placebo patients. The mean SICU stay in the placebo group was 2.5 days and in the rhAT group was 3.8 days. Mean total hospitalization time for the placebo group was 10.6 days, while total hospitalization time in the rhAT group was 11.8 days. However, there was no statistically significant difference between treatment groups in the length of SICU stay (Log-Rank test, p=0.973) or in the length of hospitalization (Log-Rank test, p=0.713).

Differences between the treatment groups were observed for the mean volume of chest tube drainage for both time periods (12 and 24 hours post-operatively). In both cases, rhAT patients had larger mean volumes of chest tube drainage than placebo patients. The mean chest tube drainage volume during the period 12 hours post-operatively was 71 mL/hr for the placebo cohort and 124 mL/hr for the rhAT cohort. During the period 24 hours post-operatively, mean chest tube drainage volume was 52 mL/hr for the placebo cohort and 86 mL/hr for the rhAT cohort. This difference was statistically significant between treatment groups for both the period 12 hours post-operatively (Rank Sum test, p=0.045) and during the period 24 hours post-operatively (Rank Sum test, p=0.030).

With regard to safety, rhAT treatment appeared to result in a slight increase in SICU stay and length of hospitalization, and a moderate increase in the amount of chest tube drainage, compared with placebo treatment. However, the treatment differences observed were not statistically significant (p>0.05). Furthermore, these results need to be interpreted with caution, as no attempt was made to standardize patient care across the 11 study sites.

Also included among these safety observations were the incidences of significant post-operative events (cardiac or neurological, invasive therapeutic cardiac procedures, rehospitalizations, or operations), administration of additional blood products, and administration of protamine. These findings are displayed in Table 32.

TABLE 32
Incidence of Other Patient Outcome Measures Related
to Safety-Specific Events (Safety Population)

| | | Treatment Group | | |
|---|---|---|---|---|
| | | | rhAT | Placebo |
| Parameter | Statistic | | n = 27 | n = 27 |

| | | | | | |
|---|---|---|---|---|---|
| Occurrence of | n (%) | 3 | (11) | 1 | (4) |
| Post-op Cardiac | | | | | |
| or Neurological | | | | | |
| Events | | | | | |
| Occurrence of | n (%) | 0 | (0) | 0 | (0) |
| Post-op Invasive | | | | | |
| Cardiac Procedures | | | | | |
| Occurrence of | n (%) | 4 | (15) | 1 | (4) |
| Rehospitalization | | | | | |
| or Post-discharge | | | | | |
| Operations | | | | | |

Administration of Packed Red Blood Cells

| | | | | | |
|---|---|---|---|---|---|
| Total | n (%) | 23 | (85) | 27 | (100) |
| Pre-Bypass | n (%) | 2 | (7) | 5 | (19) |
| Initiation | | | | | |
| On Bypass - | n (%) | 4 | (15) | 4 | (15) |
| Preprotamine Ad. | | | | | |
| Protamine Ad. - | n (%) | 4 | (15) | 4 | (15) |
| End of Surgery | | | | | |
| Within 24 Hrs. | n (%) | 20 | (74) | 20 | (74) |
| After Surgery | | | | | |

Administration of Platelets

| | | | | | |
|---|---|---|---|---|---|
| Total | n (%) | 7 | (26) | 7 | (26) |
| Pre-Bypass | n (%) | 1 | (4) | 0 | (0) |
| Initiation | | | | | |
| On Bypass - | n (%) | 0 | (0) | 0 | (0) |
| Preprotamine Ad. | | | | | |
| Protamine Ad. - | n (%) | 2 | (7) | 2 | (7) |
| End of Surgery | | | | | |
| Within 24 Hrs. | n (%) | 5 | (19) | 5 | (19) |
| After Surgery | | | | | |

Administration of Fresh Frozen Plasma

| | | | | | |
|---|---|---|---|---|---|
| Total | n (%) | 13 | (48) | 23 | (85) |
| Pre-Bypass | n (%) | 3 | (11) | 16 | (59) |
| Initiation | | | | | |
| On Bypass - | n (%) | 1 | (4) | 5 | (19) |
| Preprotamine Ad. | | | | | |
| Protamine Ad. - | n (%) | 3 | (11) | 3 | (11) |
| End of Surgery | | | | | |
| Within 24 Hrs. | n (%) | 12 | (44) | 6 | (22) |
| After Surgery | | | | | |

Administration of Other Blood Products

| | | | | | |
|---|---|---|---|---|---|
| Total | n (%) | 13 | (48) | 9 | (33) |
| Pre-Bypass | n (%) | 2 | (7) | 1 | (4) |
| Initiation | | | | | |
| On Bypass - | n (%) | 1 | (4) | 1 | (4) |
| Preprotamine Ad. | | | | | |
| Protamine Ad. - | n (%) | 2 | (7) | 1 | (4) |
| End of Surgery | | | | | |
| Within 24 Hrs. | n (%) | 13 | (48) | 6 | (22) |
| After Surgery | | | | | |

| | | | |
|---|---|---|---|
| Total Protamine | Mean | 434 | 368 |
| Administered* | (mg) | | |

*Means for total protamine administered are for the rhAT without FFP and the Placebo with FFP cohorts only. All other parameters include all cohorts.
N = 25 for rhAT group; status of protamine administration is unknown for 2 rhAT patients.

There was no difference between treatment groups in any of these parameters that would suggest a safety concern for treatment with rhAT. No patient in either group underwent an invasive therapeutic cardiac procedure post-operatively. Four rehospitalizations occurred in the rhAT group and one in the placebo group. In all cases, these rehospitalizations involved cardiac events or symptoms of cardiovascular disease (congestive heart failure, edema, atrial fibrillation, excess fluids, dyspnea). The treatment differences in the occurrence of cardiac/neurological events and rehospitalizations were not statistically significant (Barnard's Unconditional Exact test, p=0.360 and 0.235, respectively).

During the period from pre-bypass initiation to within 24 hours post surgery, significantly more placebo patients (85%) than rhAT patients (48%) received fresh frozen plasma (Barnard's Unconditional Exact test, p<0.001). Also during this time period, significantly more placebo patients (100%) than rhAT patients (85%) received transfusions of packed red blood cells (Barnard's Unconditional Exact test, p=0.041). No statistically significant differences were noted between treatment groups in the numbers of patients who received platelets or other blood products after the initiation of CPB (Barnard's Unconditional Exact test, p=1.000 and 0.406, respectively). The rhAT without FFP cohort had a larger mean total dose of protamine administered than the placebo with FFP cohort (434 mg vs. 368 mg), but the difference was not statistically significant (ANOVA, p=0.135).

The data displayed in Table 32 should be interpreted cautiously, because no attempt was made to standardize hospital procedures relating to patient care, and no consideration was given to the potential variability in physician skill.

Safety Conclusions

Adverse events, laboratory abnormalities, and other safety-related events observed across both the rhAT and placebo treatment groups were considered in the context of the study population's serious underlying disease(s). In addition, consideration was given to the fact that the events reported are known to be associated with the surgical procedures performed. These procedures, as well as the many concurrent medications that patients were receiving prior to, during, and subsequent to surgery, made it difficult to interpret the severity and relationship of safety-related events to the administration of rhAT.

Differences in the incidence of adverse events between treatment groups are likely due to normal and potentially wide variations that would be expected to occur in the patient population studied. Indeed, a statistically significant difference between the rhAT and placebo treatment groups was not observed for any of the most frequently occurring (≧10% incidence) adverse events. No adverse event was considered by the study investigators to be definitely or probably related to rhAT administration. Possible relationship to study medication was reported in association with adverse events experienced by only 6 patients in the rhAT group and 1 patient in the placebo group (all events were hemorrhage NOS). The distribution of all adverse events by severity (mild, moderate, or severe) was similar in each treatment group, except for a tendency for more reports of severe unrelated adverse events in the rhAT group.

Two deaths occurred during the study, both in the rhAT group. Neither death was considered related to treatment with rhAT. Ten rhAT patients and seven placebo patients reported one or more serious adverse events each. One SAE (hemorrhage NOS) was considered possibly related to rhAT administration; this event of “persistent bleeding” could also have been reasonably associated with the surgical procedure.

Among the adverse events associated with bleeding, hemorrhage NOS was the most frequently reported event in both treatment groups. The event occurred more frequently among rhAT patients than placebo patients, but this difference was not statistically significant. In addition, thrombocytopenia occurred only in one placebo patient, while coagulation disorder and hematoma occurred only in one rhAT patient each. Adverse events associated with bleeding were reported as SAEs for four rhAT patients (3 hemorrhage NOS, 1 coagulation disorder) and for three placebo patients (3 hemorrhage NOS). Bleeding-related adverse events were not associated with supra-normal plasma AT III levels.

Blood chemistry, hematology, and urinalysis results did not appear to reveal any safety concerns associated with rhAT treatment. No statistically significant differences (p>0.05) were observed between the two treatment groups in the percentages of patients who had laboratory abnormalities meeting the ECOG grade 3+ criteria. Only one patient in each treatment group had abnormal laboratory values that were reported as serious adverse events; these events were considered not related to administration of the study medication. Overall, many factors likely confounded interpretation of the blood chemistry, hematology, and urinalysis results. The most relevant of these may have been the fact that the majority of blood and urine samples were collected shortly after surgery during the early stages of patient recovery, a period when effects on laboratory values would have been most significant.

No significant treatment effect on vital signs or ECG results was observed within either treatment group. ELISA testing for rhAT antibody formation after administration of study medication was negative for all patients who received rhAT. Among the various other safety-related parameters monitored during the study, rhAT treatment appeared to result in a slight increase in SICU stay and length of hospitalization. Compared with placebo treatment, this difference was not statistically significant and did not suggest a safety concern for treatment with rhAT. Furthermore, from pre-bypass initiation to within 24 hours post surgery, significantly more placebo patients than rhAT patients received transfusions of fresh frozen plasma and packed red blood cells. However mean chest tube drainage volume was significantly greater among patients who received. rhAT compared to patients who received placebo during both the 12-hour postoperative period and the 24-hour postoperative period. No statistically significant or clinically relevant differences were noted between treatment groups for the remaining safety-related parameters (i.e., post-operative cardiac or neurological events, invasive therapeutic cardiac procedures, rehospitalizations or operations, or administration of platelets or other blood products, or protamine administration).

Safety results of Phase I clinical studies of rhAT, as well as the biochemical and functional similarity of rhAT to human AT III, indicate that the rhAT of the invention can be safely administered intravenously in AT III-depleted patients. Overall, in this study, intravenous administration of rhAT at a dose of 75 U/kg in patients undergoing elective cardiac surgery requiring sternotomy and cardiopulmonary bypass (CPB) appeared to be safe, when compared to administration of a placebo control and to relieve and/or lessen negative neurocognitive outcomes.

Although not prospectively proposed in this study, the Inventors investigated other biochemical markers of hemostatic activation, including prothrombin fragment 1.2 and antithrombin-thrombin complex. These investigations were carried out at the same time points using the same plasma samples from which D-Dimer, fibrin monomer, and AT III activity were determined. Prothrombin fragment 1.2 is generated when prothrombin is converted to thrombin, and thus serves as a marker of thrombin generation. Thrombin-antithrombin complex, a stable complex formed when AT III inhibits activated thrombin, serves as a marker for the dynamic between thrombin generation and thrombin inhibition. Compared to placebo patients, patients who received rhAT showed significant inhibition of the generation of these markers at the preprotamine time point in patients undergoing CPB. This is an indication of decreased thrombin generation, and therefore indicates a reduced risk of clot formation in the CPB circuit, systemic thrombosis, and bleeding post-operatively.

While gross clot formation in the extracorporeal circuit rarely occurs in current clinical practice, inhibition of the activation of the hemostatic system has important clinical implications. Activation of the hemostatic system during CPB is implicated in the generation of cerebral microemboli, one of the proposed causes of stroke and neurologic deficit often observed after CPB. Similarly, coronary, pulmonary, and intracardiac thrombosis has also been reported during cardiac surgery. Post-operative bleeding may result from consumption of labile coagulation factors secondary to thrombin activation during CPB. Although this Phase 3 study was not designed to demonstrate these clinical outcome endpoints due to the large number of patients required for differential determinations, rhAT administration clearly suppressed activation of the hemostatic system of patients on CPB.

Additional benefits of improved heparin anticoagulation management were observed among patients who received rhAT. Only 3 of 26 (12%) rhAT treated patients compared to 10 of 27 (37%) placebo treated patients required additional heparin to achieve adequate anticoagulation before the initiation of CPB. Accordingly, rhAT administration according to the current invention saves operating room time, since clinical practice dictates taking the time to draw a blood sample and monitor the ACT after each heparin administration. Patients receiving rhAT also required less total heparin during CPB. Lower overall heparin administration during CPB requires less protamine for reversal and has been associated with a decreased incidence of heparin rebound, improving patient clinical outcomes, improving efficiency and lowering hospitalization costs.

Administration of rhAT at a dose of 75 U/kg was well tolerated by heparin resistant patients undergoing CPB. There were no statistically significant differences in the incidence of adverse experiences or the incidence of clinically significant abnormal laboratory parameters noted between the two treatment groups. Furthermore, no rhAT treated patients developed antibodies to rhAT. The interpretation of hemorrhage-related adverse events was confounded by the multiple etiologies of bleeding in the setting of heart surgery and CPB. While it is conceivable that supra-normal AT III activity after rhAT administration might induce bleeding, AT III activity level data and FFP administration data do not support this hypothesis. On the other hand, mean chest tube drainage was significantly greater among patients treated with rhAT. This observation is not totally unexpected in view of provision having not been made to monitor and reduce heparin administration in the presence of normalized AT III activity or to closely monitor the effectiveness of heparin reversal with protamine during the postoperative period. Had these measures been taken, it is likely that there would have been no difference between treatment groups in mean chest tube drainage.

In summary, during cardiopulmonary bypass, blood comes into contact with the artificial surface of the bypass circuit, which results in nonspecific activation of the hemostatic system. Without anticoagulation, clots would be formed in the bypass circuit and could result in circuit occlusion. In addition, activation products would be released into the systemic circulation, and the hemostatis system would be depleted of factors and platelets.

To prevent these events, the prior art has utilized heparin alone to accelerate the action of antithrombin in the blood, reducing thrombin generation, thrombin activity, and fibrin generation in the bypass circuit and systemically. Inadequate anticoagulation during CPB potentially leads to an increased risk of clotting in the bypass circuit, thrombosis in the patient, and post-operative bleeding due to the consumption of hemostatic factors. However, heparin resistance may lead to inadequate anticoagulation during CPB associated with the risks described above.

The administration of rhAT administration suppressed increases in D-Dimer, prothrombin fragment 1.2, and antithrombin-thrombin complex normally associated with CPB, providing better control of hemostatic activation. The current invention was the first to utilize rhAT in patients with heparin resistance undergoing cardiopulmonary bypass. Heparin resistance in this setting was caused by AT III deficiency in the vast majority of cases. Administration of rhAT, 75 U/kg, restored AT III activity to normal range, which led to improved heparin sensitivity, adequate anticoagulation for CPB, and no need for treatment with FFP in the majority of cases. In contrast, AT III activity declined further from baseline in placebo treated patients, requiring treatment with FFP and other measures to obtain adequate anticoagulation before initiation of CPB. The administration of rhAT resulted in significant inhibition of coagulation markers of thrombin generation compared to placebo treated patients. Clinically, these findings translate into a reduced risk of clotting in the bypass circuit, thrombosis in the patient, and post-operative bleeding due to the consumption of hemostatic factors. Finally, rhAT at a dose of 75 U/kg was well tolerated in the setting of heparin resistant patients undergoing cardiopulmonary bypass.

Materials and Methods

Transgenic Goats & Cattle

The herds of pure- and mixed-breed scrapie-free Alpine, Saanen and Toggenburg dairy goats used as cell and cell line donors for this study were maintained under Good Agricultural Practice (GAP) guidelines. Similarly, cattle used should be maintained under Good Agricultural Practice (GAP) guidelines and be certified to originate from a scrapie and bovine encephalitis free herd.

Pregnancy and Perinatal Care.

For goats, pregnancy was determined by ultrasonography starting on day 25 after the first day of standing estrus. Does were evaluated weekly until day 75 of gestation, and once a month thereafter to assess fetal viability. For the pregnancy that continued beyond 152 days, parturition was induced with 5 mg of PGF2α (Lutalyse, Upjohn). Parturition occurred within 24 hours after treatment. Kids were removed from the dam immediately after birth, and received heat-treated colostrum within 1 hour after delivery. Time frames appropriate for other ungulates with regard to pregnancy and perinatal care (e.g., bovines) are known in the art.

Cloned Animals.

The present invention also includes a method of cloning a genetically engineered or transgenic mammal, by which a desired gene is inserted, removed or modified in the differentiated mammalian cell or cell nucleus prior to insertion of the differentiated mammalian cell or cell nucleus into the enucleated oocyte.

Also provided by the present invention are mammals obtained according to the above method, and the offspring of those mammals. The present invention is preferably used for cloning caprines or bovines but could be used with any mammalian species. The present invention further provides for the use of nuclear transfer fetuses and nuclear transfer and chimeric offspring in the area of cell, tissue and organ transplantation.

Suitable mammalian sources for oocytes include goats, sheep, cows, pigs, rabbits, guinea pigs, mice, hamsters, rats, primates, etc. Preferably, the oocytes will be obtained from ungulates, and most preferably goats or cattle. Methods for isolation of oocytes are well known in the art. Essentially, this will comprise isolating oocytes from the ovaries or reproductive tract of a mammal, e.g., a goat. A readily available source of ungulate oocytes is from hormonally induced female animals.

For the successful use of techniques such as genetic engineering, nuclear transfer and cloning, oocytes may preferably be matured in vivo before these cells may be used as recipient cells for nuclear transfer, and before they can be fertilized by the sperm cell to develop into an embryo. Metaphase II stage oocytes, which have been matured in vivo, have been successfully used in nuclear transfer techniques. Essentially, mature metaphase II oocytes are collected surgically from either non-super ovulated or super ovulated animals several hours past the onset of estrus or past the injection of human chorionic gonadotropin (hCG) or similar hormone.

Moreover, it should be noted that the ability to modify animal genomes through transgenic technology offers new alternatives for the manufacture of recombinant proteins. The production of human recombinant pharmaceuticals in the milk of transgenic farm animals solves many of the problems associated with microbial bioreactors (e.g., lack of post-translational modifications, improper protein folding, high purification costs) or animal cell bioreactors (e.g., high capital costs, expensive culture media, low yields). The current invention enables the use of transgenic production of biopharmaceuticals, transgenic proteins, plasma proteins, and other molecules of interest in the milk or other bodily fluid (i.e., urine or blood) of transgenic animals homozygous for a desired gene.

According to an embodiment of the current invention when multiple or successive rounds of transgenic selection are utilized to generate a cell or cell line homozygous for more than one trait such a cell or cell line can be treated with compositions to lengthen the number of passes a given cell line can withstand in in vitro culture. Telomerase would be among such compounds that could be so utilized.

The use of living organisms as the production process means that all of the material produced will be chemically identical to the natural product. In terms of basic amino acid structures this means that only L-optical isomers, having the natural configuration, will be present in the product. Also the number of wrong sequences will be negligible because of the high fidelity of biological synthesis compared to chemical routes, in which the relative inefficiency of coupling reactions will always produce failed sequences. The absence of side reactions is also an important consideration with further modification reactions such as carboxy-terminal amidation. Again, the enzymes operating in vivo give a high degree of fidelity and stereospecificity which cannot be matched by chemical methods. Finally the production of a transgenic protein of interest in a biological fluid means that low-level contaminants remaining in the final product are likely to be far less toxic than those originating from a chemical reactor.

As previously mentioned, expression levels of three grams per liter of ovine milk are well within the reach of existing transgenic animal technology. Such levels should also be achievable for the recombinant proteins contemplated by the current invention.

In the practice of the present invention, obesity related transgenic proteins are produced in the milk of transgenic animals. The human recombinant protein of interest coding sequences can be obtained by screening libraries of genomic material or reverse-translated messenger RNA derived from the animal of choice (such as cattle or mice), or through appropriate sequence databases such as NCBI, genbank, etc. These sequences along with the desired polypeptide sequence of the transgenic partner protein are then cloned into an appropriate plasmid vector and amplified in a suitable host organism, usually E. coli. The DNA sequence encoding the peptide of choice can then be constructed, for example, by polymerase chain reaction amplification of a mixture of overlapping annealed oligonucleotides.

After amplification of the vector, the DNA construct would be excised with the appropriate 5′ and 3′ control sequences, purified away from the remains of the vector and used to produce transgenic animals that have integrated into their genome the desired obesity related transgenic protein. Conversely, with some vectors, such as yeast artificial chromosomes (YACs), it is not necessary to remove the assembled construct from the vector; in such cases the amplified vector may be used directly to make transgenic animals. In this case obesity related refers to the presence of a first polypeptide encoded by enough of a protein sequence nucleic acid sequence to retain its biological activity, this first polypeptide is then joined to a the coding sequence for a second polypeptide also containing enough of a polypeptide sequence of a protein to retain its physiological activity. The coding sequence being operatively linked to a control sequence which enables the coding sequence to be expressed in the milk of a transgenic non-human placental mammal.

A DNA sequence which is suitable for directing production to the milk of transgenic animals carries a 5′-promoter region derived from a naturally-derived milk protein and is consequently under the control of hormonal and tissue-specific factors. Such a promoter should therefore be most active in lactating mammary tissue. According to the current invention the promoter so utilized can be followed by a DNA sequence directing the production of a protein leader sequence which would direct the secretion of the transgenic protein across the mammary epithelium into the milk. At the other end of the transgenic protein construct a suitable 3′-sequence, preferably also derived from a naturally secreted milk protein, and may be added to improve stability of mRNA. An example of suitable control sequences for the production of proteins in the milk of transgenic animals are those from the caprine beta casein promoter.

The production of transgenic animals can now be performed using a variety of methods. The method preferred by the current invention is nuclear transfer.

Milk Specific Promoters.

The transcriptional promoters useful in practicing the present invention are those promoters that are preferentially activated in mammary epithelial cells, including promoters that control the genes encoding milk proteins such as caseins, beta-lacto globulin (Clark et al., (1989) BIO/TECHNOLOGY 7: 487-492), whey acid protein (Gorton et al. (1987) BIO/TECHNOLOGY 5: 1183-1187), and lactalbumin (Soulier et al., (1992) FEBS LETTS. 297: 13). Casein promoters may be derived from the alpha, beta, gamma or kappa casein genes of any mammalian species; a preferred promoter is derived from the goat beta casein gene (DiTullio, (1992) BIO/TECHNOLOGY 10:74-77). The milk-specific protein promoter or the promoters that are specifically activated in mammary tissue may be derived from either cDNA or genomic sequences. Preferably, they are genomic in origin.

DNA sequence information is available for all of the mammary gland specific genes listed above, in at least one, and often several organisms. See, e.g., Richards et al., J. BIOL. CHEM. 256, 526-532 (1981)(α-lactalbumin rat); Campbell et al., NUCLEIC ACIDS RES. 12, 8685-8697 (1984)(rat WAP); Jones et al., J. BIOL. CHEM. 260, 7042-7050 (1985)(rat β-casein); Yu-Lee & Rosen, J. BIOL. CHEM. 258, 10794-10804 (1983) (rat γ-casein); Hall, BIOCHEM. J. 242, 735-742 (1987)(α-lactalbumin human); Stewart, NUCLEIC ACIDS RES. 12, 389 (1984) (bovine αs1 and κ casein cDNAs); Gorodetsky et al., GENE 66, 87-96 (1988)(bovine β casein); Alexander et al., EUR. J. BIOCHEM. 178, 395-401 (1988) (bovine κ casein); Brignon et al., FEBS LETT. 188, 48-55 (1977)(bovine αS2 casein); Jamieson et al., GENE 61, 85-90 (1987), Ivanov et al., BIOL. CHEM. Hoppe-Seyler 369, 425-429 (1988), Alexander et al., NUCLEIC ACIDS RES. 17, 6739 (1989) (bovine β lactoglobulin); Vilotte et al., BIOCHIMIE 69, 609-620 (1987)(bovine α-lactalbumin). The structure and function of the various milk protein genes are reviewed by Mercier & Vilotte, J. DAIRY SCI. 76, 3079-3098 (1993)(incorporated by reference in its entirety for all purposes). To the extent that additional sequence data might be required, sequences flanking the regions already obtained could be readily cloned using the existing sequences as probes. Mammary-gland specific regulatory sequences from different organisms are likewise obtained by screening libraries from such organisms using known cognate nucleotide sequences, or antibodies to cognate proteins as probes.

Signal Sequences.

Among the signal sequences that are useful in accordance with this invention are milk-specific signal sequences or other signal sequences which result in the secretion of eukaryotic or prokaryotic proteins. Preferably, the signal sequence is selected from milk-specific signal sequences, i.e., it is from a gene which encodes a product secreted into milk. Most preferably, the milk-specific signal sequence is related to the milk-specific promoter used in the expression system of this invention. The size of the signal sequence is not critical for this invention. All that is required is that the sequence be of a sufficient size to effect secretion of the desired recombinant protein, e.g., in the mammary tissue. For example, signal sequences from genes coding for caseins, e.g., alpha, beta, gamma or kappa caseins, beta lactoglobulin, whey acid protein, and lactalbumin are useful in the present invention. The preferred signal sequence is the goat β-casein signal sequence.

Signal sequences from other secreted proteins, e.g., proteins secreted by liver cells, kidney cell, or pancreatic cells can also be used.

Amino-Terminal Regions of Secreted Proteins.

The efficacy with which a non-secreted protein is secreted can be enhanced by inclusion in the protein to be secreted all or part of the coding sequence of a protein which is normally secreted. Preferably the entire sequence of the protein which is normally secreted is not included in the sequence of the protein but rather only a portion of the amino terminal end of the protein which is normally secreted. For example, a protein which is not normally secreted is fused (usually at its amino terminal end) to an amino terminal portion of a protein which is normally secreted.

Preferably, the protein which is normally secreted is a protein which is normally secreted in milk. Such proteins include proteins secreted by mammary epithelial cells, milk proteins such as caseins, beta lacto globulin, whey acid protein, and lactalbumin. Casein proteins include alpha, beta, gamma or kappa casein genes of any mammalian species. A preferred protein is beta casein, e.g., a goat beta casein. The sequences which encode the secreted protein can be derived from either cDNA or genomic sequences. Preferably, they are genomic in origin, and include one or more introns.

DNA Constructs.

The expression system or construct, described herein, can also include a 3′ untranslated region downstream of the DNA sequence coding for the non-secreted protein. This region apparently stabilizes the RNA transcript of the expression system and thus increases the yield of desired protein from the expression system. Among the 3′ untranslated regions useful in the constructs of this invention are sequences that provide a poly A signal. Such sequences may be derived, e.g., from the SV40 small t antigen, the casein 3′ untranslated region or other 3′ untranslated sequences well known in the art. Preferably, the 3′ untranslated region is derived from a milk specific protein. The length of the 3′ untranslated region is not critical but the stabilizing effect of its poly A transcript appears important in stabilizing the RNA of the expression sequence.

Optionally, the expression system or construct includes a 5′ untranslated region between the promoter and the DNA sequence encoding the signal sequence. Such untranslated regions can be from the same control region from which promoter is taken or can be from a different gene, e.g., they may be derived from other synthetic, semi-synthetic or natural sources. Again their specific length is not critical, however, they appear to be useful in improving the level of expression.

The construct can also include about 10%, 20%, 30%, or more of the N-terminal coding region of the gene preferentially expressed in mammary epithelial cells. For example, the N-terminal coding region can correspond to the promoter used, e.g., a goat B-casein N-terminal coding region.

The above-described expression systems may be prepared using methods well known in the art. For example, various ligation techniques employing conventional linkers, restriction sites etc. may be used to good effect. Preferably, the expression systems of this invention are prepared as part of larger plasmids. Such preparation allows the cloning and selection of the correct constructions in an efficient manner as is well known in the art. Most preferably, the expression systems of this invention are located between convenient restriction sites on the plasmid so that they can be easily isolated from the remaining plasmid sequences for incorporation into the desired mammal.

Prior art methods often include making a construct and testing it for the ability to produce a product in cultured cells prior to placing the construct in a transgenic animal. Surprisingly, the inventors have found that such a protocol may not be of predictive value in determining if a normally non-secreted protein can be secreted, e.g., in the milk of a transgenic animal. Therefore, it may be desirable to test constructs directly in transgenic animals, e.g., transgenic mice, as some constructs which fail to be secreted in CHO cells are secreted into the milk of transgenic animals.

Sequence Production and Modification

The invention encompasses the use of the described nucleic acid sequences and the peptides expressed therefrom in various transgenic animals. The sequences of specific molecules can be manipulated to generate proteins that retain most of their tertiary structure but are physiologically non-functional.

PCR technology may also be utilized to isolate full length cDNA sequences. For example, RNA may be isolated, following standard procedures, from an appropriate cellular or tissue source (i.e., one known, or suspected, to express a target receptor gene, such as, for example from, skin, testis, or brain tissue). A reverse transcription (RT) reaction may be performed on the RNA using an oligonucleotide primer specific for the most 5′ end of the amplified fragment for the priming of first strand synthesis. The resulting RNA/DNA hybrid may then be “tailed” using a standard terminal transferase reaction, the hybrid may be digested with RNase H, and second strand synthesis may then be primed with a complementary primer. Thus, cDNA sequences upstream of the amplified fragment may easily be isolated. For a review of cloning strategies which may be used, see e.g., Sambrook et al., 1989.

A cDNA of a mutant target gene may be isolated, for example, by using PCR. In this case, the first cDNA strand may be synthesized by hybridizing an oligo-dT oligonucleotide to mRNA isolated from tissue known or suspected to be expressed in an individual putatively carrying a mutant target allele, and by extending the new strand with reverse transcriptase. The second strand of the cDNA is then synthesized using an oligonucleotide that hybridizes specifically to the 5′ end of the normal gene. Using these two primers, the product is then amplified via PCR, optionally cloned into a suitable vector, and subjected to DNA sequence analysis through methods well known to those of skill in the art. By comparing the DNA sequence of the mutant target allele to that of the normal target allele, the mutation(s) responsible for the loss or alteration of function of the mutant target gene product can be ascertained.

Alternatively, a genomic library can be constructed using DNA obtained from an individual suspected of or known to carry the mutant target allele, or a cDNA library can be constructed using RNA from a tissue known, or suspected, to express the mutant target allele. A normal target gene, or any suitable fragment thereof, can then be labeled and used as a probe to identify the corresponding mutant target allele in such libraries. Clones containing the mutant target gene sequences may then be purified and subjected to sequence analysis according to methods well known to those of skill in the art.

Additionally, an expression library can be constructed utilizing cDNA synthesized from, for example, RNA isolated from a tissue known, or suspected, to express a mutant target allele in an individual suspected of or known to carry such a mutant allele. In this manner, gene products made by the putatively mutant tissue may be expressed and screened using standard antibody screening techniques in conjunction with antibodies raised against the normal target product.

The invention also encompasses nucleotide sequences that encode mutant target receptor protein sequences, peptide fragments of the target receptor proteins, truncated target receptor proteins, and target receptor protein fusion proteins. These include, but are not limited to nucleotide sequences encoding mutant target receptor proteins described herein; polypeptides or peptides corresponding to one or more domains of the target receptor protein or portions of these domains; truncated target receptor protein in which one or more of the domains is purposefully deleted, or a truncated non-functional target receptor protein so as to generate a purposefully dysfunctional receptor protein.

Purposefully dysfunctional receptor proteins can be made and expressed in a transgenic system to provide a composition that can bind to physiological agents that would maintain obesity or work to increase weight gain. Nucleotides encoding fusion proteins may include, but are not limited to, full length target receptor protein sequences, truncated target receptor proteins, or nucleotides encoding peptide fragments of a target receptor protein fused to an unrelated protein or peptide that will facilitate expression in a transgenic mammal or other transgenic animal expression model, such as for example, a target receptor protein domain fused to an Ig Fc domain which increases the stability and half-life of the resulting fusion protein in the bloodstream such that retains its ability to ameliorate obesity or related pathologies.

The target receptor protein amino acid sequences of the invention include the amino acid sequences presented in the sequence listings herein as well as analogues and derivatives thereof. Further, corresponding target receptor protein homologues from other species are encompassed by the invention. The degenerate nature of the genetic code is well known, and, accordingly, each amino acid presented in the sequence listings, is generically representative of the well known nucleic acid “triplet” codon, or in many cases codons, that can encode the amino acid. As such, as contemplated herein, the amino acid sequences presented in the sequence listing, when taken together with the genetic code (see, pp 109, Table 4-1 of MOLECULAR CELL BIOLOGY, (1986), J. Darnell et al. eds., incorporated by reference) are generically representative of all the various permutations and combinations of nucleic acid sequences that can encode such amino acid sequences.

According to a preferred embodiment of the invention random mutations can be made to target gene DNA through the use of random mutagenesis techniques well known to those skilled in the art with the resulting mutant target receptor proteins tested for activity, site-directed mutations of the target receptor protein coding sequence can be engineered to generate mutant target receptor proteins with the same structure but with limited physiological function, e.g., alternate function, and/or with increased half-life. This can be accomplished using site-directed mutagenesis techniques well known to those skilled in the art.

One starting point for such activities is to align the disclosed human sequences with corresponding gene/protein sequences from, for example, other mammals in order to identify specific amino acid sequence motifs within the target gene that are conserved between different species. Changes to conserved sequences can be engineered to alter function, signal transduction capability, or both. Alternatively, where the alteration of function is desired, deletion or non-conservative alterations of the conserved regions can also be engineered.

Other mutations to the target protein coding sequence can be made to generate target proteins that are better suited for expression, scale-up, etc. in the host cells chosen. For example, cysteine residues can be deleted or substituted with another amino acid in order to eliminate disulfide bridges.

While the target proteins and peptides can be chemically synthesized, large sequences derived from a target protein and full length gene sequences can be advantageously produced by recombinant DNA technology using techniques well known in the art for expressing nucleic acid containing target protein gene sequences and/or nucleic acid coding sequences. Such methods can be used to construct expression vectors containing appropriate transcriptional and translational control signals. These methods include, for example, in vitro recombinant DNA techniques, synthetic techniques, and in vivo genetic recombination.

Transgenic Mammals.

Preferably, the DNA constructs of the invention are introduced into the germ-line of a mammal. For example, one or several copies of the construct may be incorporated into the genome of a mammalian embryo by standard transgenic techniques known in the art.

Any non-human mammal can be usefully employed in this invention. Mammals are defined herein as all animals, excluding humans, which have mammary glands and produce milk. Preferably, mammals that produce large volumes of milk and have long lactating periods are preferred. Preferred mammals are cows, sheep, goats, mice, oxen, camels and pigs. Of course, each of these mammals may not be as effective as the others with respect to any given expression sequence of this invention. For example, a particular milk-specific promoter or signal sequence may be more effective in one mammal than in others. However, one of skill in the art may easily make such choices by following the teachings of this invention.

In an exemplary embodiment of the current invention, a transgenic non-human animal is produced by introducing a transgene into the germline of the non-human animal. Transgenes can be introduced into embryonal target cells at various developmental stages. Different methods are used depending on the stage of development of the embryonal target cell. The specific line(s) of any animal used should, if possible, be selected for general good health, good embryo yields, good pronuclear visibility in the embryo, and good reproductive fitness.

The litters of transgenic mammals may be assayed after birth for the incorporation of the construct into the genome of the offspring. Preferably, this assay is accomplished by hybridizing a probe corresponding to the DNA sequence coding for the desired recombinant protein product or a segment thereof onto chromosomal material from the progeny. Those mammalian progeny found to contain at least one copy of the construct in their genome are grown to maturity. The female species of these progeny will produce the desired protein in or along with their milk. Alternatively, the transgenic mammals may be bred to produce other transgenic progeny useful in producing the desired proteins in their milk.

In accordance with the methods of the current invention for transgenic animals a transgenic primary cell line (from either caprine, bovine, ovine, porcine or any other non-human vertebrate origin) suitable for somatic cell nuclear transfer is created by transfection of the transgenic protein nucleic acid construct of interest (for example, a mammary gland-specific transgene(s) targeting expression of a transgenic protein to the mammary gland). The transgene construct can either contain a selection marker (such as neomycin, kanamycin, tetracycline, puromycin, zeocin, hygromycin or any other selectable marker) or be co-transfected with a cassette able to express the selection marker in cell culture.

Transgenic females may be tested for protein secretion into milk, using any of the assay techniques that are standard in the art (e.g., Western blots or enzymatic assays).

The invention provides expression vectors containing a nucleic acid sequence described herein, operably linked to at least one regulatory sequence. Many such vectors are commercially available, and other suitable vectors can be readily prepared by the skilled artisan. “Operably linked” or “operatively linked” is intended to mean that the nucleic acid molecule is linked to a regulatory sequence in a manner which allows expression of the nucleic acid sequence by a host organism. Regulatory sequences are art recognized and are selected to produce the encoded polypeptide or protein. Accordingly, the term “regulatory sequence” includes promoters, enhancers, and other expression control elements which are described in Goeddel, GENE EXPRESSION TECHNOLOGY: METHODS IN ENZYMOLOGY 185, (Academic Press, San Diego, Calif. (1990)). For example, the native regulatory sequences or regulatory sequences native to the transformed host cell can be employed.

It should be understood that the design of the expression vector may depend on such factors as the choice of the host cell to be transformed and/or the type of protein desired to be expressed. For instance, the polypeptides of the present invention can be produced by ligating the cloned gene, or a portion thereof, into a vector suitable for expression in either prokaryotic cells, eukaryotic cells or both. (A LABORATORY MANUAL, 2nd Ed., ed. Sambrook et al. (Cold Spring Harbor Laboratory Press, 1989) Chapters 16 and 17)).

Following selection of colonies recombinant for the desired nucleic acid construct, cells are isolated and expanded, with aliquots frozen for long-term preservation according to procedures known in the field. The selected transgenic cell-lines can be characterized using standard molecular biology methods (PCR, Southern blotting, FISH). Cell lines carrying nucleic acid constructs of the obesity related transgenic protein of interest, of the appropriate copy number, generally with a single integration site (although the same technique could be used with multiple integration sites) can then be used as karyoplast donors in a somatic cell nuclear transfer protocol known in the art. Following nuclear transfer, and embryo transfer to a recipient animal, and gestation, live transgenic offspring are obtained.

Typically this transgenic offspring carries only one transgene integration on a specific chromosome, the other homologous chromosome not carrying an integration in the same site. Hence the transgenic offspring is heterozygous for the transgene, maintaining the current need for at least two successive breeding cycles to generate a homozygous transgenic animal.

Animal Promoters

Useful promoters for the expression of obesity related in mammary tissue include promoters that naturally drive the expression of mammary-specific polypeptides, such as milk proteins, although any promoter that permits secretion of obesity related into milk can be used. These include, e.g., promoters that naturally direct expression of whey acidic protein (WAP), alpha S1-casein, alpha S2-casein, beta-casein, kappa-casein, beta-lactoglobulin, alpha-lactalbumin (see, e.g., Drohan et al., U.S. Pat. No. 5,589,604; Meade et al., U.S. Pat. No. 4, 873,316; and Karatzas et al., U.S. Pat. No. 5,780,009), and others described in U.S. Pat. No. 5,750,172. Whey acidic protein (WAP; Genbank Accession No. X01153), the major whey protein in rodents, is expressed at high levels exclusively in the mammary gland during late pregnancy and lactation (Hobbs et al., J. BIOL. CHEM. 257:3598-3605, 1982). For additional information on desired mammary gland-specific promoters, see, e.g., Richards et al., J. BIOL. CHEM. 256:526-532, 1981 (α-lactalbumin rat); Campbell et al., NUCLEIC ACIDS RES. 12:8685-8697, 1984 (rat WAP); Jones et al., J. BIOL. CHEM. 260:7042-7050, 1985 (rat β-casein); Yu-Lee & Rosen, J. BIOL. CHEM. 258:10794-10804, 1983 (rat γ-casein); Hall, BIOCHEM. J. 242:735-742, 1987 (human α-lactalbumin); Stewart, NUCLEIC ACIDS RES. 12:3895-3907, 1984 (bovine α-s1 and κ-casein cDNAs); Gorodetsky et al., GENE 66:87-96, 1988 (bovine β-casein); Alexander et al., EUR. J. BIOCHEM. 178:395-401, 1988 (bovine κ-casein); Brignon et al., FEBS LETT. 188:48-55, 1977 (bovine α-S2 casein); Jamieson et al., GENE 61:85-90, 1987, Ivanov et al., BIOL. CHEM. Hoppe-Seyler 369:425-429, 1988, and Alexander et al., NUCLEIC ACIDS RES. 17:6739, 1989 (bovine β-lactoglobulin); and Vilotte et al., BIOCHIMIE 69:609-620, 1987 (bovine α-lactalbumin). The structure and function of the various milk protein genes are reviewed by Mercier & Vilotte, J. DAIRY SCI. 76:3079-3098, 1993.

If additional flanking sequences are useful in optimizing expression, such sequences can be cloned using the existing sequences as probes. Mammary-gland specific regulatory sequences from different organisms can be obtained by screening libraries from such organisms using known cognate nucleotide sequences, or antibodies to cognate proteins as probes.

Useful signal sequences for expression and secretion of obesity related into milk are milk-specific signal sequences. Desirably, the signal sequence is selected from milk-specific signal sequences, i.e., from a gene which encodes a product secreted into milk. Most desirably, the milk-specific signal sequence is related to a milk-specific promoter described above. The size of the signal sequence is not critical for this invention. All that is required is that the sequence be of a sufficient size to effect secretion of a target transgenic protein of use in the treatment of obesity, e.g., in the mammary tissue. For example, signal sequences from genes coding for caseins, e.g., alpha, beta, gamma, or kappa caseins, beta lactoglobulin, whey acidic protein, and lactalbumin are useful in the present invention. Signal sequences from other secreted proteins, e.g., proteins secreted by liver cells, kidney cell, or pancreatic cells can also be used.

Useful promoters for the expression of a recombinant polypeptide transgene in urinary tissue are the uroplakin and uromodulin promoters (Kerr et al., NAT. BIOTECHNOL. 16:75-79, 1998; Zbikowska, et al., BIOCHEM. J. 365:7-11, 2002; and Zbikowski et al., TRANSGENIC RES. 11:425-435, 2002), although any promoter that permits secretion of the transgene product into urine may be used.

A useful promoter for the expression and secretion of obesity related into blood by blood-producing or serum-producing cells (e.g., liver epithelial cells) is the albumin promoter (see, e.g., Shen et al., DNA 8:101-108, 1989; Tan et al., DEV. BIOL. 146:24-37, 1991; McGrane et al., TIBS 17:40-44, 1992; Jones et al., J. BIOL. CHEM. 265:14684-14690, 1990; and Shimada et al., FEBS LETTERS 279:198-200, 1991), although any promoter that permits secretion of the transgene product into blood may be used. The native alpha-fetoprotein promoter can also be used (see, e.g., Genbank Accession Nos.: AB053574; AB053573; AB053572; AB053571; AB053570; and AB053569). Useful promoters for the expression of obesity related in semen are described in U.S. Pat. No. 6,201,167. Useful avian-specific promoters are the ovalbumin promoter and the apo-B promoter.

Another three grams is produced in the liver (serum lipoproteins) and deposited in the egg yolk. In addition, since birds do not typically recognize mammalian proteins immunologically because of their evolutionary distance from mammals, the expression of obesity related in birds is less likely to have any deleterious effect on the viability and health of the bird.

Other promoters that are useful in the methods of the invention include inducible promoters. Generally, recombinant proteins are expressed in a constitutive manner in most eukaryotic expression systems. The addition of inducible promoters or enhancer elements provides temporal or spatial control over expression of the transgenic proteins of interest, and provides an alternative mechanism of expression. Inducible promoters include heat shock protein, metallothionien, and MMTV-LTR, while inducible enhancer elements include those for ecdysone, muristerone A, and tetracycline/doxycycline.

Therapeutic Uses.

The combination herein is preferably employed for in vitro use in treating these tissue cultures. The combination, however, is also be effective for in vivo applications. Depending on the intended mode of administration in vivo the compositions used may be in the dosage form of solid, semi-solid or liquid such as, e.g., tablets, pills, powders, capsules, gels, ointments, liquids, suspensions, or the like. Preferably the compositions are administered in unit dosage forms suitable for single administration of precise dosage amounts. The compositions may also include, depending on the formulation desired, pharmaceutically acceptable carriers or diluents, which are defined as aqueous-based vehicles commonly used to formulate pharmaceutical compositions for animal or human administration. The diluent is selected so as not to affect the biological activity of the human recombinant protein of interest. Examples of such diluents are distilled water, physiological saline, Ringer's solution, dextrose solution, and Hank's solution. The same diluents may be used to reconstitute lyophilized a human recombinant protein of interest. In addition, the pharmaceutical composition may also include other medicinal agents, pharmaceutical agents, carriers, adjuvants, nontoxic, non-therapeutic, non-immunogenic stabilizers, etc. Effective amounts of such diluent or carrier will be amounts which are effective to obtain a pharmaceutically acceptable formulation in terms of solubility of components, biological activity, etc.

The compositions herein may be administered to human patients via oral, parenteral or topical administrations and otherwise systemic forms for anti-melanoma and anti-breast cancer treatment.

Bacterial Expression.

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include E. coli., Bacillus subtilis, Salmonella typhimurium and various species within the genera Pseudomonas, Streptomyces, and Staphylococcus, although others may, also be employed as a matter of choice. In a preferred embodiment, the prokaryotic host is E. coli.

Bacterial vectors may be, for example, bacteriophage-, plasmid- or cosmid-based. These vectors can comprise a selectable marker and bacterial origin of replication derived from commercially available plasmids typically containing elements of the well known cloning vector pBR322 (ATCC 37017). Such commercial vectors include, for example, GEM 1 (Promega Biotec, Madison, Wis., USA), pBs, phagescript, PsiX174, pBluescript SK, pBs KS, pNH8a, pNH16a, pNH18a, pNH46a (Stratagene); pTrc99A, pKK223-3, pKK233-3, pKK232-8, pDR540, and pRIT5 (Pharmacia). A preferred vector according to the invention is THE Pt7I expression vector.

These “backbone” sections are combined with an appropriate promoter and the structural sequence to be expressed. Bacterial promoters include lac, T3, T7, lambda PR or PL, trp, and ara. T7 is a preferred bacterial promoter.

Following transformation of a suitable host strain and growth of the host strain to an appropriate cell density, the selected promoter is de-repressed/induced by appropriate means (e.g., temperature shift or chemical induction) and cells are cultured for an additional period. Cells are typically harvested by centrifugation, disrupted by physical or chemical means, and the resulting crude extract retained for further purification.

Eukaryotic Expression Vectors

Various mammalian cell culture systems can also be employed to express recombinant proteins. Examples of mammalian expression systems include selected mouse L cells, such as thymidine kinase-negative (TK) and adenine phosphoribosul transferase-negative (APRT) cells. Other examples include the COS-7 lines of monkey kidney fibroblasts, described by Gluzman, CELL 23:175 (1981), and other cell lines capable of expressing a compatible vector, for example, the C127, 3T3, CHO, HeLa and BHK cell lines. In particular, as regards yeasts, there may be mentioned yeasts of the genus Saccharomyces, Kluyveromyces, Pichia, Schwanniomyces, or Hansenula. Among the fungi capable of being used in the present invention, there may be mentioned more particularly Aspergillus ssp, or Trichoderma ssp.

Mammalian expression vectors will comprise an origin of replication, a suitable promoter and enhancer, and also any necessary ribosome binding sites, polyadenylation site, splice donor and acceptor sites, transcriptional termination sequences, and 5′ flanking non-transcribed sequences. DNA sequences derived from the SV40 viral genome, for example, SV40 origin, early promoter, enhancer, splice, and polyadenylation sites may be used to provide the required non-transcribed genetic elements.

Mammalian promoters include beta-casein, beta-lactoglobulin, whey acid promoter others include: HSV thymidine kinase, early and late SV40, LTRs from retrovirus, and mouse metallothionein-1. Exemplary mammalian vectors include pWLneo, pSV2cat, pOG44, pXT1, pSG (Stratagene) pSVK3, pBPV, pMSG, and pSVL (Pharmacia). In a preferred embodiment, the mammalian expression vector is pUCIG-MET. Selectable markers include CAT (chloramphenicol transferase).

The nucleotide sequences which can be used within the framework of the present invention can be prepared in various ways. Generally, they are obtained by assembling, in reading phase, the sequences encoding each of the functional parts of the polypeptide. The latter may be isolated by the techniques of persons skilled in the art, and for example directly from cellular messenger RNAs (mRNAs), or by recloning from a complementary DNA (cDNA) library, or alternatively they may be completely synthetic nucleotide sequences. It is understood, furthermore, that the nucleotide sequences may also be subsequently modified, for example by the techniques of genetic engineering, in order to obtain derivatives or variants of the said sequences.

Fluorescence In Situ Hybridization (FISH) Analysis.

Standard culture and preparation procedures are used to obtain metaphase and interphase nuclei from cultured cells derived from animals carrying the desirable transgene. Nuclei are deposited onto slides and were hybridized with a digoxigenin-labeled probe derived from a construct containing 8 kb of the genomic sequence for the obesity related protein of interest. Bound probe was amplified using a horseradish peroxidase-conjugated antibody and detected with tyramide-conjugated fluorescein isothiocyanate (FITC, green fluorochrome). Nuclei were counterstained with 4′,6-diamidino-2-phenylindole (DAPI, blue dye). FISH images were obtained using MetaMorph software.

Therapeutic Compositions.

The proteins of the present invention can be formulated according to known methods to prepare pharmaceutically useful compositions, whereby the inventive molecules, or their functional derivatives, are combined in admixture with a pharmaceutically acceptable carrier vehicle. Suitable vehicles and their formulation, inclusive of other human proteins, e.g., human serum albumin, are described, for example, in order to form a pharmaceutically acceptable composition suitable for effective administration, such compositions will contain an effective amount of one or more of the proteins of the present invention, together with a suitable amount of carrier vehicle.

Pharmaceutical compositions for use in accordance with the present invention may be formulated in conventional manner using one or more physiologically acceptable carriers or excipients. Thus, the obesity related molecules and their physiologically acceptable salts and solvate may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they maybe presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the composition may take the form of tablets or lozenges formulated in conventional manner.

For administration by inhalation, the obesity related molecules for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethan-e, carbon dioxide or other suitable gas. In the case of a pressurized aerosol the dosage unit may be determined by providing a valve to deliver a metered amount. Capsules and cartridges of, e.g. gelatin for use in an inhaler or insufflator may be formulated containing a powder mix of the compound and a suitable powder base such as lactose or starch.

The obesity related transgenic proteins of the invention may be formulated for parenteral administration by injection, e.g., by bolus injection or continuous intransgenic. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the obesity related molecules may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Treatment Methods.

The inventive therapeutic methods according to the invention generally utilize the obesity related proteins identified above. The domains of the transgenic proteins share the ability to specifically target a specific tissue and/or augment an immune response to targeted tissue. A typical method, accordingly, involves binding a receptor of a targeted cell to the receptor-antagonizing domain of the transgenic protein and/or stimulating a T-cell dependent immune response.

Therapeutic methods involve administering to a subject in need of treatment a therapeutically effective amount of a transgenic protein. “Therapeutically effective” is employed here to denote the amount of transgenic proteins that are of sufficient quantity to inhibit or reverse a disease condition (e.g., reduce or inhibit cancer growth). Some methods contemplate combination therapy with known cancer medicaments or therapies, for example, chemotherapy (preferably using compounds of the sort listed above) or radiation. The patient may be a human or non-human animal. A patient typically will be in need of treatment when suffering from a cancer characterized by increased levels of receptors that promote cancer maintenance or proliferation.

Administration during in vivo treatment may be by any number of routes, including parenteral and oral, but preferably parenteral. Intracapsular, intravenous, intrathecal, and intraperitoneal routes of administration may be employed, generally intravenous is preferred. The skilled artisan will recognize that the route of administration will vary depending on the disorder to be treated.

Determining a therapeutically effective amount specifically will depend on such factors as toxicity and efficacy of the medicament. Toxicity may be determined using methods well known in the art and found in the foregoing references. Efficacy may be determined utilizing the same guidance in conjunction with the methods described below in the Examples. A pharmaceutically effective amount, therefore, is an amount that is deemed by the clinician to be toxicologically tolerable, yet efficacious. Efficacy, for example, can be measured by the induction or substantial induction of T lymphocyte cytotoxicity at the targeted tissue or a decrease in mass of the targeted tissue. Suitable dosages can be from about 1 mg/kg to 10 mg/kg.

The foregoing is not intended to have identified all of the aspects or embodiments of the invention nor in any way to limit the invention. The accompanying drawings, which are incorporated and constitute part of the specification, illustrate embodiments of the invention, and together with the description, serve to explain the principles of the invention.

All publications and patent applications mentioned in this specification are herein incorporated by reference to the same extent as if each independent publication or patent application is specifically indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

### LITERATURE CITED AND INCORPORATED BY REFERENCE

1. Alberio R, et al., (2001). Mammalian Oocyte Activation: Lessons from the Sperm and Implications for Nuclear Transfer, INT J DEV BIOL 2001; 45: 797-809.
2. Andrew M. J. et al., Neuropsychological Dysfunction After Minimally Invasive Direct Coronary Artery Bypass Grafting, ANNALS OF THORACIC SURGERY, 66(5):1611-7, (1998).
3. Arroyo, A. et al., (2000). In Vivo Roles of Integrins During Leukocyte Development and Traffic: Insights From the Analysis of Mice Chimeric For α₅, α_{V}, and α₄ Integrins. THE JI 165: 4667-4675.
4. Arrowsmith J. E. et al., Central Nervous System Complications of Cardiac Surgery, BRITISH JOURNAL OF ANAESTHESIA, 84(3):378-93, (2000).
5. Baguisi A,(1999) et al., Production of Goats by Somatic Cell Nuclear Transfer, NATURE BIOTECH; 17: 456-461.
6. Buss M. I. et al., Cardiopulmonary Bypass, Rewarming, and Central Nervous System Dysfunction, ANNALS OF THORACIC SURGERY, 61(5):1423-7, (1996).
7. Carrascal Y. et al., Neurological Complications After Cardiopulmonary Bypass: An Update, EUROPEAN NEUROLOGY, 41(3):128-34, (1999).
8. Despotis, G. J., et al., The Impact Of heparin Concentration And Activated Clotting Time Monitoring On Blood Conservation, J. THOR. CARD. SURG., 110:46-54, (1995).
9. Despotis, G. J., et al., Antithrombin III During Cardiac Surgery: Effect On Response Of Activated Clotting Time To Heparin And Relationship To Markers Of Hemostatic Activation, ANESTHESIA AND ANALGESIA, (1997).
10. Despotis G J, et al., Anticoagulation Monitoring During Cardiac Surgery—A Review Of Current And Emerging Techniques, ANESTHESIOLOGY, Vol. 91, (1999).
11. Edmunds, T. et al., Transgenically Produced Human Antithrombin: Structural and Functional Comparison to Human Plasma-Derived Antithrombin, BLOOD, 12(June 15): 4561-71 (1998).
12. Engelman R. M., et al., Influence of Cardiopulmonary Bypass Perfusion Temperature on Neurologic and Hematologic Function After Coronary Artery Bypass Grafting, ANNALS OF THORACIC SURGERY, 67(6):1547-1555, (1999).
13. Georgiadis D. et al., Predictive Value of S-100 beta and Neuron-Specific Enolase Serum Levels for Adverse Neurologic Outcome after Cardiac Surgery, JOURNAL OF THORACIC & CARDIOVASCULAR SURGERY, 119(1):138-47, (2000).
14. Harrison M. J., Neurologic Complications of Coronary Artery Bypass Grafting: Diffuse or Focal Ischemia?, ANNALS OF THORACIC SURGERY, 59(5): 1356-8, (1995).
15. Herczeg L. et al., Morphological Damage to the Central Nervous System (CNS) Following Open Heart Surgery, FORENSIC SCIENCE INTERNATIONAL, 79(2):103-11, (1996).
16. Herrmann M. et al., Neurobehavioral Outcome Prediction after Cardiac Surgery: Role of Neurobiochemical Markers of Damage to Neuronal and Glial Brain Tissue, STROKE, 31(3):645-50, (2000).
17. Inoue K. et al., Incidence and Risk Factors of Perioperative Cerebral Complications: Heart Transplantation Compared to Coronary Artery Bypass Grafting and Valve Surgery, JOURNAL OF CARDIOVASCULAR SURGERY, 39(2):201-8, (1998).
18. Janssen D. P., et al., Predictors of Neurological Morbidity After Coronary Artery Bypass Surgery, EUR. J ADOLESC HEALTH, 16(3):397-8 (1999).
19. Johnsson P., Markers of Cerebral Ischemia After Cardiac Surgery, JOURNAL OF CARDIOTHORACIC & VASCULAR ANESTHESIA, 10(1): 120-6, (1996).
20. Johnsson P. et al., Cerebral Complications After Cardiac Surgery Assessed by S-100 and NSE Levels in Blood, JOURNAL OF CARDIOTHORACIC & VASCULAR ANESTHESIA, 9(6):694-9, (1995).
21. Kasinathan P, (2001) et al., Effect of Fibroblast Donor Cell Age and Cell Cycle on Development of Bovine Nuclear Transfer Embryos In Vitro, BIOL REPROD.; 64(5): 1487-1493.
22. Levy J. H. et al., Recombinant Antithrombin: Production and Role in Cardiovascular Disorder, SEMINARS IN THROMBOSIS AND HEMOSTASIS, 27(4): 405-16, (2001).
23. Meng L, et al., (1997) Rhesus Monkeys Produced by Nuclear Transfer, BIOL REPROD. August; 57(2):454-9.
24. Mickleborough L. L. et al., Risk Factors For Stroke In Patients Undergoing Coronary Artery Bypass Grafting, JOURNAL OF THORACIC & CARDIOVASCULAR SURGERY, 112(5):1250-58, (1996).
25. Mortensen, R, et al., (1992) Production of Homozygous Mutant ES Cells with a Single Targeting Construct, MOL. CELL. BIOL. 12, 2391-2395.
26. Murkin J M., The Role of CPB Management In Neurobehavioral Outcomes After Cardiac Surgery, ANNALS OF THORACIC SURGERY, 59(5):1308-11, (1995).
27. Murkin J M., Etiology snd Incidence of Brain Dysfunction After Cardiac Surgery, JOURNAL OF CARDIOTHORACIC & VASCULAR ANESTHESIA. 13(4 Suppl 1):12-7; discussion 36-37, (1999).
28. Nagy, A, et al., (1996) Targeted Mutagenesis: Analysis of Phenotype Without Germ-Line Transmission. J. CLIN. INVEST. 97: 1360-1365.
29. Newman M. F. et al., Cardiopulmonary Bypass And The Central Nervous System: Potential For Cerebral Protection, JOURNAL OF CLINICAL ANESTHESIA. 8(3 Suppl):53S-60S, (1996).
30. Newman M. F., et al., Cerebral Physiologic Effects Of Burst Suppression Doses Of Propofol During Nonpulsatile Cardiopulmonary Bypass, ANESTHESIA & ANALGESIA. 81(3):452-57, (1995).
31. Newman M. F. et al., (2001) Longitudinal Assessment of Neurocognitive Function After Coronary-Artery Bypass Surgery, NEW ENG. J. MED. 344(6), 395-402.
32. Ongeri E M, et al., (2001) Development of Goat Embryos After In VitroFertilization and Parthenogenetic Activation by Different Methods, THERIOGENOLOGY June 1;55(9):1933-45.
33. Rasmussen L. S. et al., Do Blood Levels of Neuron-Specific Enolase and S-100 Protein Reflect Cognitive Dysfunction After Coronary Artery Bypass? ACTA ANAESTHESIOLOGICA SCANDINAVICA, 43(5):495-500, (1999).
34. Roach G. W., et al., Ineffectiveness of Burst Suppression Therapy in Mitigating Perioperative Cerebrovascular Dysfunction, ANESTHESIOLOGY, 90(5):1255-64, (1999).
35. REMINGTON'S PHARMACEUTICAL SCIENCES (16^{th} ed., Osol, A., editor; Mack, Easton Press. (1980)).
36. Sambrook et al., MOLECULAR CLONING—A LABORATORY MANUAL, Cold Spring Harbor Laboratory Press, (2nd Edition) (1989).
37. Selnes O. A., et al., Neurobehavioural Sequelae of Cardiopulmonary Bypass, LANCET. 353(9164):1601-06, (1999).
38. Soloway, H. B., Christiansen, T. W., Heparin Anticoagulation During Cardiac Surgery in an AT III Deficient Patient, AM. J CLIN. PATHOL. 73: 723-5, 1980.
39. Stump, D A, et al. Cerebral Emboli and Cognitive Outcome After Cardiac Surgery, JOURNAL OF CARDIOTHORACIC AND VASCULAR ANESTHESIA, 10 (1): 113-19, (1996).
40. Subramanian V A., Less Invasive Arterial CABG On A Beating Heart, ANNALS OF THORACIC SURGERY, 63(6 Suppl):S68-71, (1997).
41. Taylor K M., Central Nervous System Effects Of Cardiopulmonary Bypass, ANNALS OF THORACIC Surgery. 66(5 Suppl):S20-4; discussion S25-8, (1998).
42. Utley, J. R., Pathophysiology of Cardiopulmonary Bypass: Current Issues. JOURNAL OF CARDIAC SURGERY 5.3: 177-89, 1990.
43. Videm V. et al., Heparin-Coated Cardiopulmonary Bypass Equipment. I. Biocompatibility Markers And Development Of Complications In A High-Risk Population, JOURNAL OF THORACIC & CARDIOVASCULAR SURGERY. 11 7(4):794-802, (1999).
44. Wilmut I, et al., (2002) Somatic Cell Nuclear Transfer, NATURE 10;419(6907):583-6.
45. Wilmut I, et al., (1997) Viable Offspring Derived From Fetal and Adult Mammalian Cells, NATURE February 27;385(6619):810-3.
46. Zou X, et al., (2002) Generation of Cloned Goats (Capra Hircus) from Transfected Foetal Fibroblast Cells, The Effect of Donor Cell Cycle, MOL REPROD DEV.; 61: 164-172.

1. A method for the treatment of prophylaxis of a neurocognitive disorder experienced by a surgical patient comprising administering an effective amount of antithrombin.

2. The method of claim 1, wherein said antithrombin is human recombinant antithrombin.

3. The method of claim 2, wherein said antithrombin is produced by a transgenic non-human animal or a plant.

4. The method of claim 3, wherein said antithrombin is produced by a transgenic non-human mammal.

5. The method of claim 3, wherein said antithrombin is produced by an ungulate.

6. The method of either claims 3 or 5, wherein said transgenic animal is an ungulate selected from the group consisting of bovine, ovine, porcine, equine, caprine and buffalo.

7. The method of claim 4, wherein said transgenic non-human mammal provides a donor differentiated mammalian cell to be used as a source of donor nuclei or donor cell nucleus and that such donor cell nucleus is from an adult non-human mammalian somatic cell.

8. The method of claim 3, wherein said non-human animal is a rodent.

9. The method of claim 4, wherein said transgenic non-human mammal provides a donor differentiated mammalian cell to be used as a source of donor nuclei or donor cell nucleus is a non-quiescent somatic cell or a nucleus isolated from said non-quiescent somatic cell.

10. The resultant offspring of the methods of claims 3 or 6.

11. The method of claim 1 further comprising using a second pharmaceutical agent.

12. The method of claim 11 wherein said second pharmaceutical agent is a compound selected from the group consisting of: urokinase, PF4, alpha-fetoprotein, C-1 esterase inhibitor, tPa, decorin, interferon, transferrin conjugates with biologically active peptides or fragments thereof, human serum albumin, thiopental sodium, thrombin, heparin, blood Factor X, blood Factor VIII, as well as monoclonal antibodies.

13. The method of claim 2 wherein said antithrombin is rhAT and is produced in milk.

14. The resultant milk derived from the offspring of the methods of claim 13.

15. The method of claim 1 wherein said surgical patient has undergone or is undergoing a surgical procedure selected from the group consisting of: heart transplantation, CABG, valve surgery and CABG simultaneously with valve surgery.

16. The method of claim 1 wherein said surgical patient has undergone a surgical procedure that has caused a central nervous system injury.

17. A method of treating a neurocognitive disorder or other disease condition medically related to said neurocognitive disorder comprising administering to a mammal in need of such treatment a therapeutically effective amount of a human recombinant antithrombin (rhAT) or a prodrug thereof or a pharmaceutically acceptable salt of said compound or of said prodrug.

18. A method as recited in claim 17 wherein the amount of said rhAT is about 0.01 mg/kg/day to about 50 mg/kg/day.

19. A recombinant protein as recited in claim 17 wherein the mammal is a human.

20. A recombinant DNA vector comprising the nucleic acid sequence of the transgenic protein of claim 17.

21. A host cell transformed with said recombinant DNA vector of claim 20.

22. The method of claim 17 wherein said disease condition is a neurocognitive disorder.

23. The method of claim 17 wherein said disease condition is an embolism.

24. The method of claim 17 wherein said disease condition is an parieto-occipital watershed area injury.

25. The method of claim 17 wherein said disease condition is a visuoconstruction related deficiency.

26. A method as recited in claim 17 wherein the amount of said rhAT is about 0.01 mg/kg/day to about 50 mg/kg/day in conjunction with the administration an effective amount of a second pharmaceutical compound.

27. The method as recited in claim 26 wherein said second pharmaceutical compound is sodium pental.

28. The method as recited in claim 27 wherein the amount of said sodium pental is about 0.01 mg/kg/day to about 50 mg/kg/day.

29. The method of claim 2 wherein said rhAT administered to a surgical patient has a faster plasma clearance time and an increased affinity for heparin both compared to plasma derived human antithrombin.

30. The method of claim 2 wherein the activity of said rhAT is measured by a kit comprising neuron-specific enolase (NSE) and the S-100 protein.

31. The method of claim 4 or 17, wherein said transgenic non-human mammal is generated through a nuclear microinjection procedure.
